(19) United States
(12) Patent Application Publication
(10) Pub. No.: US 2018/0353645 A1
(43) Pub. Date: Dec. 13, 2018

(54) LENS REGENERATION USING ENDOGENOUS STEM/PROGENITOR CELLS

(71) Applicants: YOUHEALTH BIOTECH, LIMITED, Grand Cayman (KY); THE REGENTS OF THE UNIVERSITY OF CALIFORNIA, Oakland, CA (US)

(72) Inventors: Kang ZHANG, San Diego, CA (US); Rui HOU, Shenyang (CN)

(21) Appl. No.: 16/060,314

(22) PCT Filed: Dec. 8, 2016
(86) PCT No.: PCT/US16/65642

§ 371 Date: Jun. 7, 2018

Related U.S. Application Data
(60) Provisional application No. 62/264,828, filed on Dec. 8, 2015.

(51) Int. Cl. A61L 27/36; A61L 27/54; A61F 9/00; A61F 9/007; A61F 9/008

(52) U.S. Cl. CPC A61L 27/3616; A61L 27/54; A61F 9/0017; A61F 9/00745; A61F 9/008; A61L 2300/414; A61B 2090/367; A61L 2400/06; A61B 2217/005; A61F 2009/0087; A61F 2009/00889; A61B 2218/007; A61L 2430/16

(57) ABSTRACT

The disclosure herein includes uses and systems for cataract removal and lens regeneration using endogenous stem cells that results in improved outcomes.

### CROSS-REFERENCE

This application claims the benefit of U.S. Provisional Application No. 62/264,828, filed Dec. 8, 2015, which the application is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Dec. 7, 2016 is named 49697-702.601 SL.txt and is 7,505 bytes in size.

### BACKGROUND OF THE DISCLOSURE

Cataract is the leading cause of blindness in the world. The visual axis, defined as the normal passage of light into the eye, may undergo visual axis opacification (VAO) due to the cataractous lens or the postoperative disorganized growth of remaining lens epithelial stem/progenitor cells (LECs), leading to vision loss. The current standard-of-care in congenital cataract involves surgical removal of the cataractous lens with a large central capsulorhexis opening and implantation of an artificial intraocular lens (IOL) to replace the missing refractive media.

### SUMMARY OF THE DISCLOSURE

According to one aspect of the present disclosure, provided herein are methods for cataract removal and lens regeneration using endogenous lens epithelial stem and progenitor cells. In some embodiments, the method comprises the steps of making a capsulorhexis opening in a peripheral area of lens anterior capsule of an eye of a subject having cataract; and removing contents of the lens, thereby preserving the lens capsule and a plurality of endogenous lens epithelial stem and progenitor cells, from which a transparent biconvex lens is regenerated. In some embodiment, the methods disclosed herein are minimally invasive.

In one refinement of the method disclosed herein, the capsulorhexis opening is about 1.0 to 2.0 mm in diameter.

In one refinement of the method disclosed herein, the capsulorhexis opening is located away from the central visual axis of the eye.

In one refinement of the method disclosed herein, the subject is an animal or human.

In one refinement of the method disclosed herein, the human is an adult or an infant.

In one refinement of the method disclosed herein, the human infant has congenital cataract.

In one refinement of the method disclosed herein, the lens epithelial stem and progenitor cells express Pax6 and Bmi-1.

In one refinement of the method disclosed herein, the method results in lowered incidents of complications selected from the group consisting of corneal edema, anterior chamber inflammation, and visual axis opacification.

Accordingly to another aspect of the present disclosure, provided herein are devices and a system to perform the new minimally invasive capsulorhexis surgery. In some embodiments, the system for performing a minimally invasive method of cataract removal comprises an imaging unit, a phacoemulsification unit for emulsifying cataract material, an aspiration unit for removing cataract material, and a biomaterial delivery unit for delivering a biomaterial composition into capsular bag via a lens capsule opening. In some embodiments, at least one of the imaging unit, phacoemulsification unit, aspiration unit, and biomaterial delivery unit are operationally connected to a computer. In some embodiment, all of the imaging unit, phacoemulsification unit, aspiration unit, and biomaterial delivery unit are operationally connected to a computer.

In one refinement of the system disclosed herein, the phacoemulsification unit comprises an ultrasound or laser probe, said probe is equipped with a tip designed to be inserted into a peripheral area of lens anterior capsule of an eye.

In one refinement of the system disclosed herein, the tip is configured to perform one or both of making an opening of about 1.0 to 2.0 mm in diameter and removing cataract from the eye.

In one refinement of the system disclosed herein, the tip is configured to prevent damage to endogenous lens epithelial stem and progenitor cells.

In one refinement of the system disclosed herein, the imaging unit employs imaging technique selected from the group consisting of 3D imaging, optical coherence tomography, MRI, CT, and ultrasound.

In one refinement of the system disclosed herein, the biomaterial composition comprises one or more of cross-linking agents, nutrients, growth factors, serum supplementation, and extracellular matrix components.

Accordingly to another aspect of the present disclosure, provided herein are methods of culturing endogenous lens epithelial progenitor cells. In some embodiments, the method comprises the steps of isolating lens epithelial progenitor cells from a subject; and culturing the lens epithelial progenitor cells on a surface coated with extracellular matrix components, wherein the progenitor cells proliferate and differentiate into lens fiber cells to form a lens.

In one refinement of the method disclosed herein, the extracellular matrix components comprise one or more molecules selected from the group consisting of mammalian amniotic membrane such as human amniotic membrane, collagen (e.g., collagen IV), fibrinogen, perlecan, laminin, fibronectin, proteoglycan, procollagens, hyaluronic acid, entactin, heparan sulfate, tenascin, poly-L-lysine, gelatin, poly-L-ornithin, platelet derived growth factor (PDGF), extracellular matrix proteins (Fischer or Life Tech), fibrinogen and thrombin sheet (Reliance Life), and Matrigel™ (BD Biosciences), human amniotic membrane, human-derived fibronectin, recombinant fibronectin matrix (Sigma), St. Louis, Mo., USA extracellular matrix produced using known recombinant DNA technology, the equivalents thereof, and combinations thereof.

In one refinement of the method disclosed herein, the progenitor cells are cultured in the presence of one or more of cross-linking agents, nutrients, growth factors, and serum supplementation.

In one refinement of the method disclosed herein, the subject is an animal or human.

In one refinement of the method disclosed herein, the isolation of lens epithelial progenitor cells comprises selecting or enriching progenitor cells that express Pax6 and Bmi-1.

Accordingly to another aspect of the present disclosure, provided herein are methods for lens regeneration using endogenous lens epithelial stem and progenitor cells. In some embodiments, the method comprises the steps of: stimulating proliferation of endogenous lens stem and progenitor cell; inducing differentiation of endogenous lens stem and progenitor cell into lens fiber cells; and facilitating maturation into an entire lens.

In one refinement of the method disclosed herein, the facilitating step is through manipulation of growth factors (such as FGFs), extracellular matrix, biomaterials, 3D printing.

In some embodiments, disclosed herein is a use of a biomaterial composition to maintain the structural integrity of a lens anterior capsule of an eye of a subject and to induce expansion of lens epithelial stem and progenitor cells in situ, wherein the biomaterial composition is administered into the lens anterior capsule through an capsulorhexis opening located at a peripheral area of the lens anterior capsule, and wherein the contents of the lens is removed prior to administration of the biomaterial composition.

In some embodiments, the biomaterial composition comprises human serum and a fibroblast growth factor (FGF).

In some embodiments, the biomaterial composition further comprises a nutrient, an additive, or a combination thereof.

In some embodiments, the nutrient comprises a composition of amino acids and optionally one or more nutrients.

In some embodiments, the additive comprises calcium chloride, potassium chloride, magnesium sulfate, sodium chloride, monosodium phosphate, potassium phosphate, sodium bicarbonate, sodium phosphate, or a combination thereof.

In some embodiments, the biomaterial composition is administered in a volume sufficient to replace the volume lost due to the removal of the contents of the lens from the lens anterior capsule.

In some embodiments, the capsulorhexis opening is about 1.0 to 2.0 mm in diameter.

In some embodiments, the capsulorhexis opening is about 1.0 to 1.5 mm in diameter.

In some embodiments, the capsulorhexis opening is located away from the central visual axis of the eye.

In some embodiments, the subject has cataract.

In some embodiments, the subject is an animal or human.

In some embodiments, the human is aged 18 or older.

In some embodiments, the human is aged 17 or younger.

In some embodiments, the human has a pediatric cataract.

In some embodiments, the human is an adult or an infant.

In some embodiments, the human infant has congenital cataract.

In some embodiments, cataract is removed.

In some embodiments, the lens epithelial stem and progenitor cells express Pax6 and/or Bmi-1.

In some embodiments, the use does not involve an implantation of an artificial intraocular lens (IOL).

In some embodiments, the use results in reduced visual axis opacification (VAO) relative to a use comprising a capsulorhexis procedure comprising central capsulorhexis opening and implantation of an artificial intraocular lens.

In some embodiments, the use results in lowered incidents of complications selected from the group consisting of corneal edema, anterior chamber inflammation, and visual axis opacification.

In some embodiments, disclosed herein is a system for performing a minimally invasive method of cataract removal, comprising an imaging unit, a phacoemulsification unit for emulsifying cataract material, an aspiration unit for removing cataract material, and a biomaterial delivery unit for delivering a biomaterial composition into a capsular bag via a lens capsule opening, wherein all of the units are operationally connected to a computer.

In some embodiments, the phacoemulsification unit comprises an ultrasound or laser probe, said probe is equipped with a tip designed to be inserted into a peripheral area of lens anterior capsule of an eye.

In some embodiments, the tip is configured to perform one or both of making an opening of about 1.0 to 2.0 mm in diameter and removing cataract from the eye.

In some embodiments, the tip is configured to perform one or both of making an opening of about 1.0 to 1.5 mm in diameter and removing cataract from the eye.

In some embodiments, the tip is configured to prevent damage to endogenous lens epithelial stem and progenitor cells.

In some embodiments, the imaging unit employs imaging technique selected from the group consisting of 3D imaging, optical coherence tomography, MRI, CT, and ultrasound.

In some embodiments, the biomaterial composition comprises human serum and a fibroblast growth factor (FGF).

In some embodiments, the biomaterial composition further comprises a nutrient, an additive, or a combination thereof.

In some embodiments, the nutrient comprises a composition of amino acids and optionally one or more nutrients.

In some embodiments, the additive comprises calcium chloride, potassium chloride, magnesium sulfate, sodium chloride, monosodium phosphate, potassium phosphate, sodium bicarbonate, sodium phosphate, or a combination thereof.

In some embodiments, the biomaterial composition is administered in a volume sufficient to replace the volume lost due to the removal of the cataract material from the capsular bag.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-FIG. 1C illustrate surgical methods and lens regeneration for congenital cataract. FIG. 1A-FIG. 1B exemplify slit-lamp photography of “doughnut-like” lens regeneration at different time points after treatment using the current surgical method. Two years after surgery (FIG. 1A), the transparent regenerated lens tissue contained the sealed capsular opening with an opaque white scar at the center. The regions between the dashed circles indicated by the red arrows are the regenerated lens tissues. Four years after surgery (FIG. 1B), the capsular opening was constricted compared to that seen at two years post-surgery, indicating continued growth of the regenerated lens. There was also the complication of iridolenticular synechiae. FIG. 1C illustrate schematic diagrams of the current surgical method for pediatric cataract: the currently practiced pediatric ACCC creates an opening 6 mm in diameter at the center of the anterior capsule, removing the LECs underneath it and leaving a relatively large wound area of 28 mm². The scars formed often cause postoperative VAO. Additionally, PCCC and anterior vitrectomy are commonly performed at follow-up visits.

FIG. 2A-FIG. 2E illustrate BrdU pulse labeling of human LECs. FIG. 2A illustrate whole mount of a human lens capsule showing BrdU⁺ cells (brown) by enzymatic immunohistology and diaminobenzidine staining. FIG. 2B illustrates high magnification images of human donor lenses showing BrdU⁺ LECs. FIG. 2C illustrates bar graph showing quantification of BrdU⁺ cells. There was an age-dependent decrease in the number of BrdU⁺ cells (8 months: 38.7±10.9, 30 years: 19.0±9.4 and 40 years: 6.0±2.2, 8 months vs 40 years, *P<0.05). 3 randomly chosen fields of each capsule were used for analysis, 3 samples in each group. FIG. 2D illustrate high magnification images of whole-mount staining of human lens capsules with or without injury showed a marked increase in the number of BrdU⁺ cells after injury. FIG. 2E illustrate bar graph showing quantification of BrdU⁺ cells. The contralateral eyes from the respective donors were used as controls. Fold of change after Injury: 11.3±0.8, *P<0.05. 3 randomly chosen fields within the germinative zone of each capsule were used for analysis, 3 samples in each group. Data shown as means±s.d.

FIG. 3A-FIG. 3C illustrate lineage tracing of Pax6⁺ LECs in mice. FIG. 3A illustrate Pax6-directed GFP was expressed in mouse LEC nuclei at postnatal days P1, P14, and P30; a sagittal section of a P0-3.9-GFPCre mouse lens is shown. Blue and green represent DAPI and anti-GFP antibody fluorescence, respectively. FIG. 3B illustrate lineage tracing of Pax6⁺ LECs in ROSA^{mTmG}; P0-3.9-GFPCre mice at P1, P14, and P30 reveals that lens fiber cells express membrane GFP fluorescence; hence, PAX6⁺ LECs were able to generate lens fiber cells. FIG. 3C illustrate as an additional control, the ROSA^{mTmG} allele alone exhibits Tomato staining at sites of non-recombination. All scale bars: 100 μm.

FIG. 4A-FIG. 4C exemplify characterization and differentiation of rabbit LECs. FIG. 4A illustrate that LECs were positive for PAX6 (green) and SOX2 (red). FIG. 4B illustrates lentoid formation (green arrows) with positive αA-crystallin and β-crystallin staining on day 15 of LECs differentiation. FIG. 4C left panel: phase contrast photograph of a lentoid body on day 30; middle panel: a lentoid body demonstrating magnifying properties; right panels, photograph of Western-blot analysis (left) and quantification (right) showing a dramatic increase in expression of mature lens fiber markers αA-crystallin (2.6±0.5), β-crystallin (10.2±1.3), and γ-crystallin (2.3±0.4). n=3 biological replicates, data shown as means±s.d. All scale bars, 100 μm.

FIG. 5A-FIG. 5B illustrates characterization of human LECs. FIG. 5A illustrate cultured human fetal LECs were positive for BMI1 (green, right upper panel); co-staining of PAX6 (red) and Ki67 (green), middle panels; co-staining of SOX2 (red) and Ki67 (green), lower panels. FIG. 5B illustrates Co-staining of PAX6 (red) and SOX2 (green) of human fetal LECs. All scale bars, 100 μm.

FIG. 6A-FIG. 6D illustrate conditional deletion of Bmi-1 led to decrease in Pax6⁺ and Sox2⁺ cells and cataract formation. FIG. 6A illustrates Loss of Bmi-1 reduced the Pax6+ and Sox2+ LECs population. Representative images of H&E stained lens sections from Bmi-1^{fl/fl} control mice and Nestin-Cre;Bmi-1^{fl/fl} mice are shown (a′). Representative images of Bmi-1 (red) staining in LECs is shown (b′). Pax6 (red) and Sox2 (green) immunostaining are shown (c′). Percentage of positive Pax6 (Bmi-1^{fl/fl}: 88.5±2.9%, Nestin-Cre;Bmi-1^{fl/fl}: 2.4±2.3%) and Sox2 (Bmi-1^{fl/fl}: 82.7±3.9%, Nestin-Cre;Bmi-1^{fl/fl}: 4.9±1.5%) cells are shown (d′, *P<0.001). FIG. 6B illustrates conditional deletion of Bmi-1 led to reduced LECs proliferation. The percentage of BrdU⁺ LECs per eye is shown (2M: Bmi-1^{fl/fl}: 2.6±0.9%; Nestin-Cre;Bmi-1^{fl/fl}: 3.0±0.4%, n=4. 7M: Bmi-1^{fl/fl}: 1.5±0.2%; Nestin-Cre;Bmi-1^{fl/fl}: 0.6±0.4%, n=6. 12M: Bmi-1^{fl/fl}: 1.8±0.6%, Nestin-Cre;Bmi-1^{fl/fl}: 0.2±0.2%, n=8), 2 sections counted per eye. Statistical significance was assessed using a two-tailed Student's t-test. *P<0.05. Data are shown as mean s.d. FIG. 6C illustrates Nestin (green) staining in E13.5, E18.5, and 2-month-old wild-type mice. All scale bars, 100 μm. FIG. 6D illustrates representative images of lenses from Nestin-Cre;Bmi-1^{fl/fl} and Bmi-1^{fl/fl} control mice (a′) show that cataracts are evident in 7- and 12-month-old Nestin-Cre;Bmi-1^{fl/fl} mice (arrow). Deletion of Bmi-1 at 6 weeks of age with Nestn-CreER did not recapitulate the cataract phenotype 10 months after tamoxifen treatment (b′). H&E stained sections of the same eyes are also shown. All scale bars, 100 μm.

FIG. 7A-FIG. 7B illustrate loss of BMI-1 decreased the proliferative ability of LECs. FIG. 7A illustrates phase contrast photographs of human LECs (upper panels) and quantification of Ki67+ proliferating human fetal LECs upon BMI1 knockdown (shBMI1) compared to controls (two shRNAs gave similar results, n=3, LEC line, each shRNA experiment was repeated 3 times, P<0.05). Blue indicates DAPI staining. FIG. 7B illustrates BMI-1 was reduced by ↓3.6 fold (n=3, P<0.05). Gene expression changes in LECs were: ↑1.6 fold in PAX6, ↑1.1 fold in SOX2, 11.3 fold in C-MAF, ↑1.0 fold in E-cadherin (all n=3, P<0.05); gene expression changes in lens fiber cells: ↑1.7 fold in Filensin, ↑0.9 fold in CP49, ↓1.5 fold in CRYBA2, (two shRNAs gave similar results, all n=3, LEC line, each shRNA experiment was repeated 3 times, P<0.05).

FIG. 8A-FIG. 8C illustrate higher expression levels of Bmi1, Sox2 and Ki67 in Pax6⁺ LECs. FIG. 8A illustrates Pax6⁺-GFP⁺ LECs were observed at the germinative zone. Right panel, a section of lens of a Pax6P0-3.9-GFPCre mouse at P1. Middle and right panels: higher magnification of the framed area in the left panel. Blue indicates DAPI staining. FIG. 8B upper panel: bright field photograph showing flat mount preparation of a lens capsule of a Pax6P0-3.9-GFPCre mouse at 6 months; lens capsule materials between two red circles were dissected to enrich Pax6⁺-GFP⁺ LECs; lower panel: fluorescence image of GFP⁺ LECs from the framed area in the upper panel. AC, anterior capsule; PC, Posterior Capsule. FIG. 8C illustrates comparison of gene expression levels in Pax6⁺-GFP⁺ LECs versus GFP⁻ LECs in anterior lens capsule in 6 month old mice, increased expression of the following genes were observed (↑10.1 fold in Pax6 (P<0.005), ↑8.2 fold in Ki67 (P<0.05), ↑4.3 fold in Bmi1 (P<0.05), and ↑2.6 fold in Sox2 (P<0.05), all n=5).

FIG. 9A-FIG. 9G illustrate lens regeneration in rabbits. FIG. 9A illustrates new minimally invasive surgical method. The capsulorhexis size was decreased to 1.0-1.5 mm in diameter, resulting in a much reduced wound area of only 1.2 mm². The location of the capsulorhexis was moved to a peripheral area of the lens. FIG. 9B illustrates slit-lamp microscopy showed that one day after surgery, the anterior and posterior capsules adhered. Four to five weeks after surgery, regenerating lens tissue grew from the periphery toward the center in a curvilinear pattern. Seven weeks after surgery, regenerating lens tissue formed a transparent biconvex lens structure. FIG. 9C illustrates Fundus examination of rabbit eyes seven weeks post-surgery demonstrated that the retina was clearly visible. Fundus examination through a normal healthy lens is shown for comparison. FIG. 9D illustrates measurements of refractive diopters in rabbit eyes at different time points post-surgery (M, month; D=Diopters). Refractive diopters of the eyes increased with time after surgery, demonstrating the functionality of the regenerated lenses (ANOVA, *P<0.01). The refractive power immediately after surgery was defined as zero, 1M=0.0D, 3M=11.0±0.8 D and 5M=15.8±2.2 D, n=3 randomly selected rabbits at each time point, Data shown as means±s.d. FIG. 9E-FIG. 9F illustrate Ki67 staining in the germinative zone of normal rabbit lens (FIG. 9E) and regenerated rabbit lens 7 weeks post-surgery (FIG. 9F). Lower panels show higher magnification. FIG. 9G illustrates PAX6 (red) and BrdU (green) staining at the germinative zone of regenerated rabbit lens 7 weeks post-surgery. Scale bars, 100 μm.

FIG. 10A-FIG. 10I illustrate lens regeneration surgery in rabbits. A 3.2 mm keratome was used to make a limbus tunnel incision at the 11-12 o'clock position into the anterior chamber (FIG. 10A). The capsular opening was created by a capsulorhexis needle (FIG. 10B). A 1-2 mm diameter anterior capsulotomy was performed using the anterior continuous curvilinear capsulorhexis (ACCC) technique near the capsular opening area (yellow arrow) (FIG. 10C). A blunt needle was used to inject balanced salt solution for hydrodissection of the cortex from the anterior capsule (FIG. 10D). The cortex was removed using a phacoemulsification device (FIG. 10E). The remaining cortex was removed using irrigation and aspiration (FIG. 10F). An elbow I/A handle was used to clear the equatorial cortex (FIG. 10G). FIG. 10H-FIG. 10I illustrate that the limbus wound was sutured with an interrupted 10-0 nylon suture. The wound was found to be watertight.

FIG. 11A-FIG. 11C illustrate lens regeneration in rabbits. FIG. 11A illustrates H&E staining of regenerated lenses at different time points after surgery. At postoperative day 1, a monolayer of LECs between the anterior and posterior capsules was visible (arrowheads). At postoperative day 4, LECs proliferated and covered the posterior capsule. At postoperative day 7, LECs in the posterior capsule began to elongate and differentiate. FIG. 11B illustrate that at postoperative day 28, LECs in the posterior capsule further elongated, forming primary lens fibers. FIG. 11C illustrates the transparency and shape of regenerated lenses in rabbits. Upper panel: Slit-lamp photography of a regenerated lens at different time points after surgery. Lower panel: Schematic diagram of slit-lamp photographs in the upper panel. At day 1 after surgery, the capsular opening was clearly seen in the peripheral anterior capsule, and the area of LECs loss during surgery is indicated. At 7 weeks after surgery, loss of LECs led to adhesion between the anterior and the posterior capsule and inhibition of lens regeneration in this area.

FIG. 12A-FIG. 12B illustrate lens regeneration in macaque models after minimally invasive surgery. FIG. 12A exemplify that slit-lamp microscopy showed that the regenerating lens tissue grew from the peripheral to the central lens in a circular symmetrical pattern 2-3 months after surgery, reaching the center at 5 months post-surgery. Five months after surgery, direct illumination showed that the visual axis remained translucent. FIG. 12B illustrates Pentacam cross-sectional scanning showed formation of a biconvex structure 5 months after surgery (yellow arrowheads). Direct illumination and fundus photography showed that the visual axis remained transparent and the retina was clearly visible. (n=6)

FIG. 13A-FIG. 13C illustrate the functional characteristics of regenerated human lenses. FIG. 13A illustrates that lens thickness increased after surgery. Pentacam showed that 3 months after surgery, the regenerating lens tissue grew from the periphery of the capsular bag to the center. The sealed capsular bag was only partially filled, appearing spindle-shaped on cross-sectional scan. The fundus was clearly visible on ophthalmoscopy. Arrowheads indicate the regenerated lens structure. FIG. 13B illustrates six months after surgery, the capsular bag was filled with regenerated lens tissue and appeared biconvex on cross-sectional scan by Pentacam. The anterior-posterior capsular adhesion disappeared. The fundus could be seen clearly using an ophthalmoscope with an 18-diopter lens. FIG. 13C shows visual acuity was measured preoperatively and at 1 week, 3 months, and 6 months postoperatively. The majority of eyes in the control group underwent additional laser capsulotomy at 3 months after surgery, with visual acuity measured before and after the procedure. There was no significant difference in visual acuity between eyes that received minimally invasive surgery (n=24) and those that were treated with the current surgical technique (n=50), except at 3 months before the control group underwent laser capsulotomy (t-test, ***P<0.001). (Notes: OD=right eye, OS=left eye, OU=bilateral eyes)

FIG. 14A-FIG. 14E illustrate the functional characteristics of regenerated human lenses. FIG. 14A exemplify that lens thickness increased significantly 6 and 8 months after surgery (1.9±0.3 and 3.7±0.3 mm, separately, *P<0.01). FIG. 14B illustrates lens refractive power increased significantly 6 and 8 months after surgery (5.1±0.5 and 19.0±0.6 D, separately, *P<0.01). FIG. 14C illustrates visual acuity improved after surgery. Pairwise analysis was performed to compare visual acuity before and after surgery (OD: 2.1±0.0, 1.6±0.1, 1.3±0.1, 1.0±0.1; OS: 2.1±0.0, 1.6±0.1, 1.3±0.1, 1.0±0.1; OU: 2.1±0.1, 1.4±0.1, 1.1±0.1, 0.8±0.1; P<0.05). FIG. 14D illustrates accommodative power increased significantly from 1 week (Control OD: 0.1±0.1 D, Control OS: 0.1±0.1 D; OD: 0.1±0.1 D, OS: 0.1±0.1 D) to 8 months (Control OD: 0.2±0.1 D, Control OS: 0.2±0.1 D; OD: 2.5±0.2 D, OS: 2.5±0.2 D) postoperatively (*P<0.001). Data shows the mean±s.d. and statistical significance was assessed using a two-tailed Student's t-test. (Notes: OD=right eye, OS=left eye, OU=bilateral eyes) FIG. 14E illustrates visual axis transparency was achieved in nearly all cataractous infant eyes after minimally invasive surgery (95.8%). The scar tissue of the wound on the anterior capsule was <1.5 mm in diameter and located in the periphery, away from the visual axis. The scars were not visible unless the pupils were dilated. No disorganized tissue regeneration was observed. Compared to the current standard surgical method, the new surgical technique decreased VAO by >20-fold.

FIG. 15 illustrates minimally invasive capsulorhexis preserved LECs for lens regeneration in human infants. Top panel: Slit-lamp exam demonstrating visual axis transparency of a human infant eye 6 months after minimally invasive surgery compared to baseline (before cataract surgery). Bottom panel: Retroillumination demonstrating the reduced size of the capsulorhexis (white arrowheads).

FIG. 16A-FIG. 16B, FIG. 17, FIG. 18 and FIG. 19 exemplify using some extracellular matrix, channels, frames in tissue engineering to create a way to guide lens stem and progenitor cells to migrate, differentiate into mature lens fiber cells in the process of lens regeneration. They also show the LEC protection method disclosed herein.

FIG. 20A-FIG. 20B exemplify a clinical trial consort flowchart (FIG. 20A) and a comparison of visual acuity mean response profiles in two groups (FIG. 20B). A non-parallel pattern of mean responses between two groups was observed largely due to the vision loss at 3 month before laser surgery in the control group (left panel), whereas a parallel pattern of mean responses between two groups was observed using time points including 3 month after laser surgery (right panel); n=25 control, n=12 experimental. Data are shown as mean±s.d.

FIG. 21A-FIG. 21B illustrate loss of BMI-1 decreased the proliferative ability of LECs. FIG. 21A illustrates phase contrast photographs of human LECs (upper panels) and quantification of Ki67+ proliferating human fetal LECs upon BMI1 knockdown (shBMI1) compared to controls (two shRNAs gave similar results, n=5, P<0.05). Data shown as mean±s.d. Blue indicates DAPI staining. FIG. 21B illustrates BMI-1 was reduced by ↓3.3 fold (all n=3, P<0.05); Gene expression changes in LEC markers were: ↑1.3 fold in PAX6, ↑1.1 fold in SOX2, 11.3 fold in C-MAF, ↑1.1 fold in E-cadherin; gene expression changes in differentiated lens fiber cell markers: ↑1.6 fold in Filensin, ↑0.9 fold in CP49, ↓1.4 fold in CRYBA2. (Two different shRNAs gave similar results; n=5, P<0.05) Data shown as mean±s.d.

FIG. 22 illustrates a conceptual schematic of a system described herein.

FIG. 23 illustrates a diagram of the computer system disclosed herein.

### DETAILED DESCRIPTION OF THE INVENTION

Each year, more than 20 million cataract patients worldwide undergo treatment with lens extraction and artificial intraocular lens (IOL) implantation. In some instances, complications related to IOLs have been observed, e.g., IOL dislocation, suboptimal biocompatibility, inadequate accommodation, and poor visual outcomes. In some cases, irreversible blindness has also been observed from IOL implantation. Thus, a new strategy for treating congenital cataracts using naturally regenerated lenses is highly desirable.

In some embodiments, surgical procedures for pediatric cataract involve creating an opening about 5-6 mm in diameter at the center of the anterior capsule. The size of the opening prolongs recovery time and increases the incidence of inflammation, while wound healing may form scars and cause postoperative visual axis opacification (VAO). In some cases, the surgical procedure removes most of the anterior subcapsular lens epithelial stem/progenitor cells (LECs), of which a subpopulation may be utilized for lens regeneration. In additional cases, abnormal proliferation of residual LECs causes postoperative VAO in many cases, which requires opening of the posterior capsule, performed by either laser capsulotomy or posterior continuous curvilinear capsulorhexis (PCCC) and anterior vitrectomy. By destroying the integrity of the lens capsule and LECs, the surgical procedure greatly diminishes the possibility of lens regeneration.

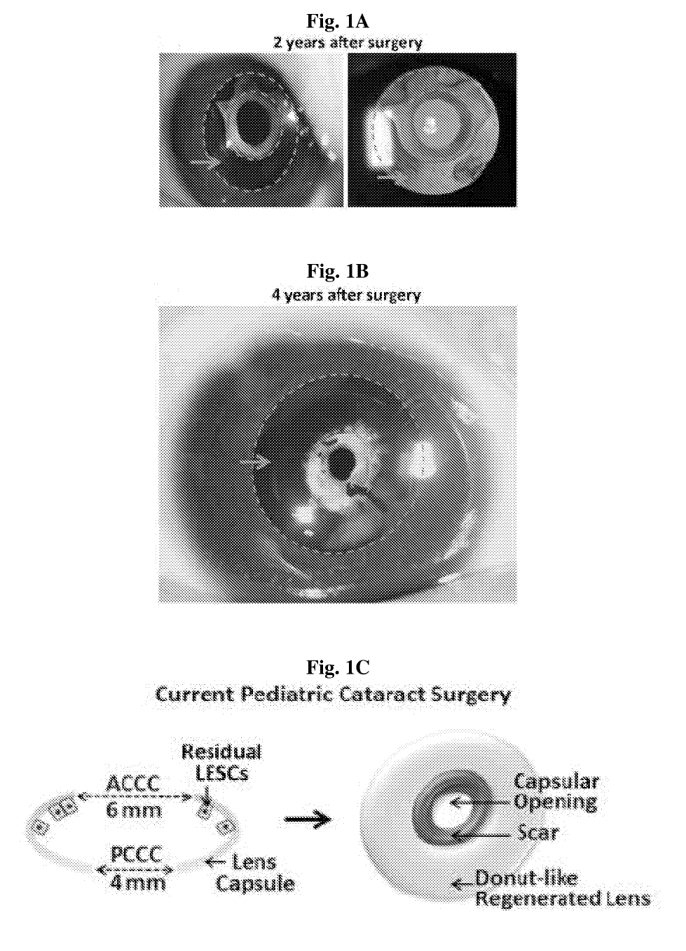

The present disclosure recognizes that stem cell therapy holds great promise in regenerative medicine. Although much attention has been focused on pluripotent stem cells and the use of their derivatives for therapeutic purposes, the present disclosure recognizes that several uncertainties, including tumorigenicity and immune rejection, have hindered their clinical application. Furthermore, the present disclosure recognizes alternatives, which is to harness the potential of endogenous progenitor cells for direct use in repair and regeneration. In the case of the ocular lens, it is recognized that successful regeneration of a complete mammalian lens with biological function has yet to be achieved, and that mechanism underlying lens regeneration remains elusive, although varying degrees of disorganized regrowth of doughnut-like lens tissues have been observed after congenital cataract removal in infants (FIG. 1A-FIG. 1B).

The present disclosure also recognizes that although artificial IOLs are widely used in pediatric cataract surgery, they are limited by complications, and that most pediatric patients continue to require some form of refractive correction such as eyeglasses after cataract surgery. Furthermore, the present disclosure recognizes that IOLs are controversial in patients younger than two years as they have not been shown to prevent strabismus or amblyopia, and normal lens refractive power is not yet fully developed at this age.

The present disclosure further recognizes that the current treatment and surgery for cataracts has limitations and poses significant risk of complications in people with cataract. Therefore, the present disclosure recognizes the need for an improved method of in situ regeneration of a functional lens.

As provided in the present disclosure, in vitro studies was performed on PAX6⁺/SOX2⁺ LECs and BMI-1 was identified as an essential factor for maintaining a LEC pool in mammalian eyes by conditional knockout experiments. The ability of LECs to differentiate into lens fiber cells in vitro was also investigated. Furthermore, in vivo animal studies was performed by first establishing a new minimally invasive capsulorhexis surgery method that differs conceptually from current practice in extracting the cataractous lens through a small wound opening, while preserving lens capsule integrity and therefore LECs as well. Using this method, lens regeneration was investigated in rabbits and macaques and a clinical trial was conducted in human infants. Functional lens regeneration was observed not only in rabbits and macaques, but also in human patients with congenital cataract. Therefore, the present disclosure provides a novel approach to lens regeneration using endogenous stem cells that results in improved outcomes.

According to some embodiments of the present disclosure, provided herein are methods for cataract removal and lens regeneration using endogenous lens epithelial progenitor cells. In some embodiments, the method comprises the steps of making a capsulorhexis opening in a peripheral area of lens anterior capsule of an eye of a subject having cataract; and removing contents of the lens, thereby preserving the lens capsule and a plurality of endogenous lens epithelial progenitor cells, from which a transparent biconvex lens is regenerated. In some embodiments, the methods disclosed herein are minimally invasive.

In some embodiments of the method disclosed herein, the capsulorhexis opening is about 1.0 to 2.0 mm in diameter.

In some embodiments of the method disclosed herein, the capsulorhexis opening is located away from the central visual axis of the eye.

In some embodiments of the method disclosed herein, the subject is an animal or human.

In some embodiments of the method disclosed herein, the human is an adult or an infant.

In some embodiments of the method disclosed herein, the human infant has congenital cataract.

In some embodiments of the method disclosed herein, the lens epithelial progenitor cells express Pax6 and Bmi-1.

In some embodiments of the method disclosed herein, the method results in lowered incidents of complications selected from the group consisting of corneal edema, anterior chamber inflammation, and visual axis opacification.

In some embodiments of the present disclosure, provided herein are devices and a system to perform the new minimally invasive capsulorhexis surgery. In some embodiments, the system for performing a minimally invasive method of cataract removal comprises an imaging unit, a phacoemulsification unit for emulsifying cataract material, an aspiration unit for removing cataract material, and a biomaterial delivery unit for delivering biomaterial into capsular bag via a lens capsule opening. In some embodiments, at least one of the imaging unit, phacoemulsification unit, aspiration unit, and biomaterial delivery unit are operationally connected to a computer. In some embodiments, all of the imaging unit, phacoemulsification unit, aspiration unit, and biomaterial delivery unit are operationally connected to a computer.

In some embodiments of the system disclosed herein, the phacoemulsification unit comprises an ultrasound or laser probe, said probe is equipped with a tip designed to be inserted into a peripheral area of lens anterior capsule of an eye.

In some embodiments of the system disclosed herein, the tip is configured to perform one or both of making an opening of about 1.0 to 2.0 mm in diameter and removing cataract from the eye.

In some embodiments of the system disclosed herein, the tip is configured to prevent damage to endogenous lens epithelial progenitor cells.

In some embodiments of the system disclosed herein, the imaging unit employs imaging technique selected from the group consisting of 3D imaging, optical coherence tomography, MRI, CT, and ultrasound.

In some embodiments of the system disclosed herein, the biomaterial composition comprises one or more of cross-linking agents, nutrients, growth factors, serum supplementation, and extracellular matrix components.

In some embodiments of the present disclosure, provided herein are methods of culturing endogenous lens epithelial progenitor cells. In some embodiments, the method comprises the steps of isolating lens epithelial progenitor cells from a subject; and culturing the lens epithelial progenitor cells on a surface coated with extracellular matrix components, wherein the progenitor cells proliferate and differentiate into lens fiber cells to form a lens.

In some embodiments of the method disclosed herein, the extracellular matrix components comprise one or more molecules selected from the group consisting of mammalian amniotic membrane such as human amniotic membrane, collagen (e.g., collagen IV), fibrinogen, perlecan, laminin, fibronectin, proteoglycan, procollagens, hyaluronic acid, entactin, heparan sulfate, tenascin, poly-L-lysine, gelatin, poly-L-ornithin, platelet derived growth factor (PDGF), extracellular matrix proteins (Fischer or Life Tech), fibrinogen and thrombin sheet (Reliance Life), and Matrigel™ (BD Biosciences), human amniotic membrane, human-derived fibronectin, recombinant fibronectin matrix (Sigma), St. Louis, Mo., USA extracellular matrix produced using known recombinant DNA technology, the equivalents thereof, and combinations thereof.

In some embodiments of the method disclosed herein, the progenitor cells are cultured in the presence of one or more of cross-linking agents, nutrients, growth factors, and serum supplementation.

In some embodiments of the method disclosed herein, the subject is an animal or human.

In some embodiments of the method disclosed herein, the isolation of lens epithelial progenitor cells comprises selecting or enriching progenitor cells that express Pax6 and Bmi-1.

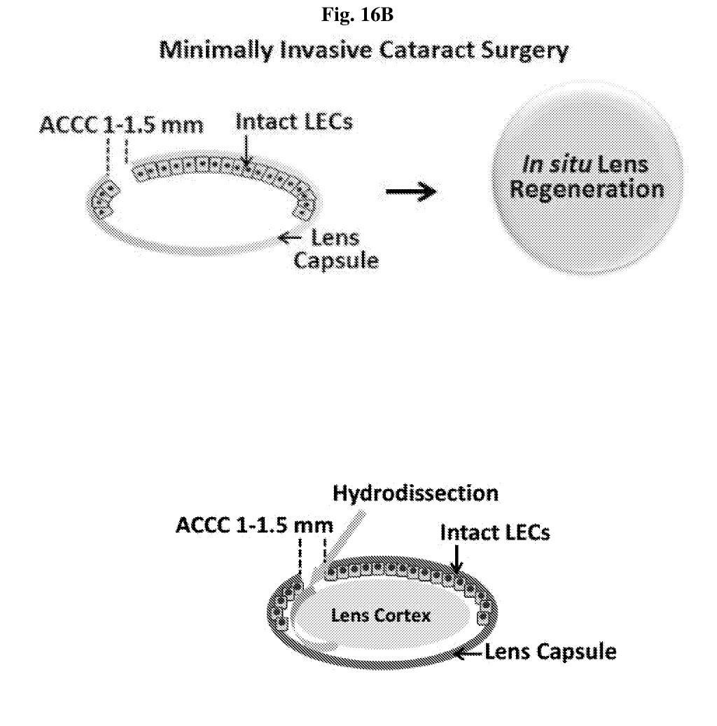

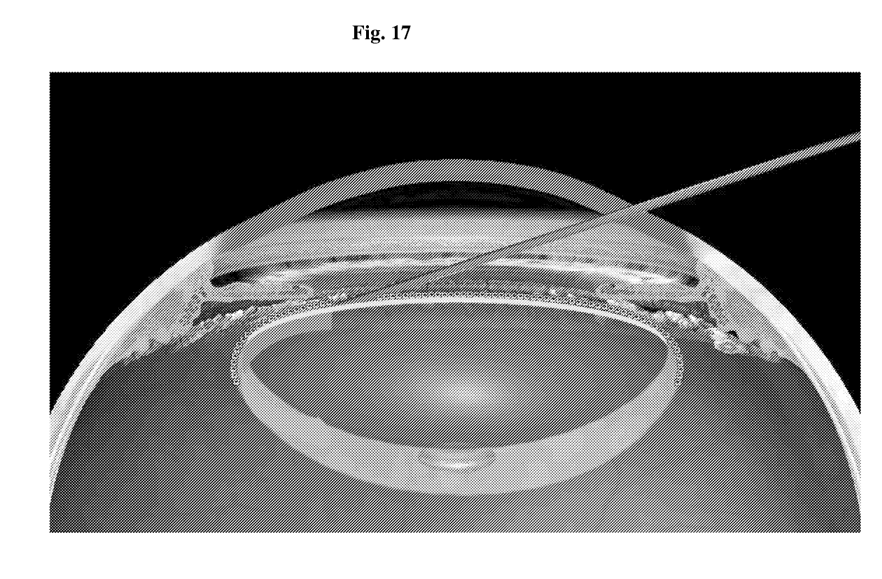

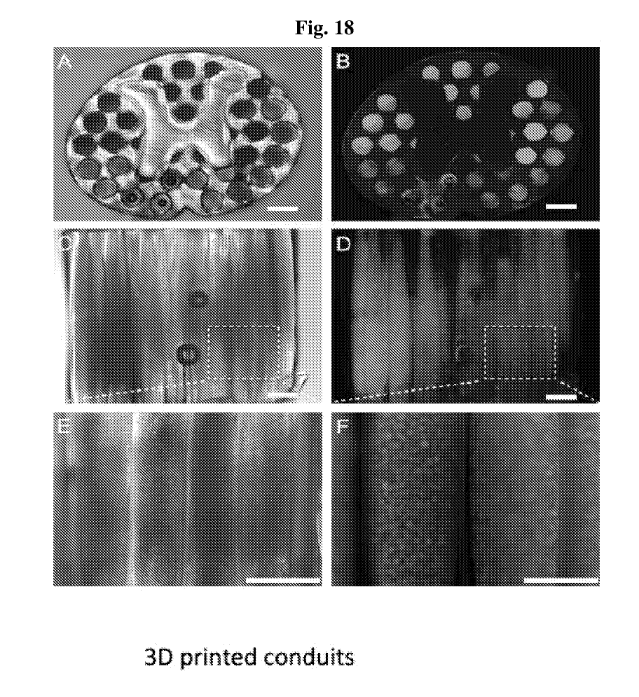

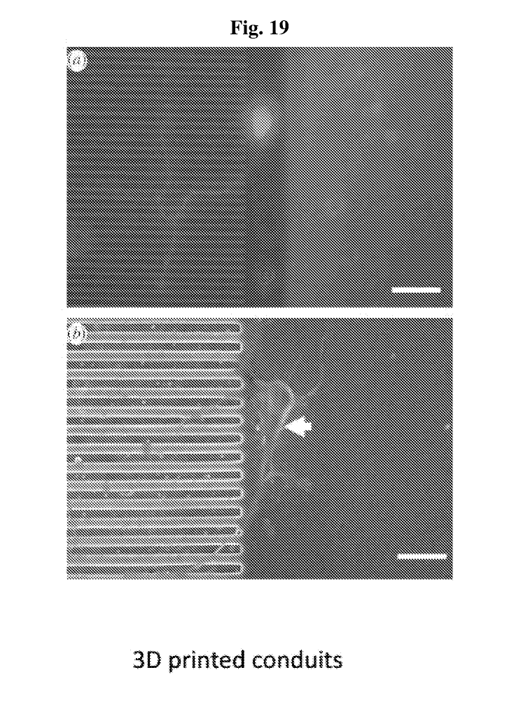

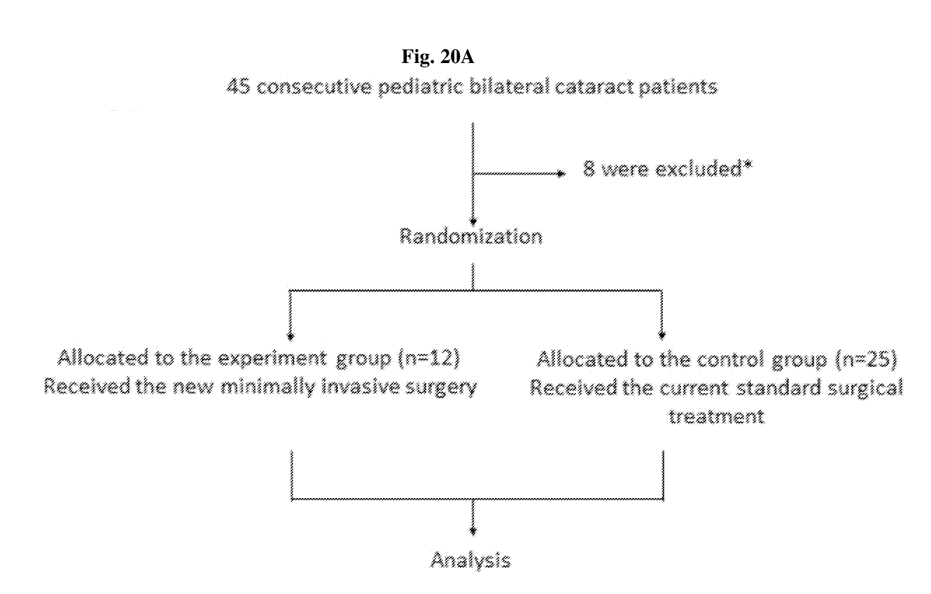

In some embodiments of the present disclosure (as shown in FIG. 16-FIG. 19), provided herein are methods for lens regeneration using endogenous lens epithelial progenitor cells. In some embodiments, the method comprises the steps of: stimulating proliferation of endogenous lens progenitor cell; inducing differentiation of endogenous lens progenitor cell into lens fiber cells; and facilitating maturation into an entire lens.

In some embodiments of the method disclosed herein, the facilitating step is through manipulation of growth factors (such as FGFs), extracellular matrix, biomaterials, or 3D printing.

### Lens Architecture and Lens Epithelia Stem/Progenitor Cells

Lens is a transparent biconvex structure that helps to focus light on the retina. In some instances, the lens belongs to the anterior segment of the eye and is connected to the ciliary body by the suspensory ligament of the lens, a ring of fibrous tissue. Posterior to the lens is the vitreous body, which along with the aqueous humor on the anterior surface, bathes the lens. In an adult human, the lens typically has a diameter of about 10 mm and an axial length of about 4 mm.

In some embodiments, the lens capsule is a smooth transparent basement membrane that surrounds the lens. In some instances, the capsule is primarily composed of collagen, with Type IV collagen and sulfated glycosaminoglycans (GAGs) as the main components. In some cases, the lens capsule is connected to the cilary body by zonular fibers.

In some instances, the lens comprises lens epithelium and lens fibers. In some cases, lens epithelium comprises simple cuboidal epithelium, which is a type of epithelium that comprises a single layer of cuboidal (cube-like) cells. In some embodiments, lens epithelium is located in the anterior portion of the lens between the lens capsule and the lens fibers, with the epithelial cells predominately located in a germinative zone, a narrow cellular region that rings the lens epithelium toward the periphery of the anterior lens surface. In some instances, newly formed cells within the germinative zone elongate and migrate along, the inner capsular surface toward the lens equator, forming new lens fiber cells as they continue to elongate and migrate posteriorly beyond the equator. In some cases, these new fiber cells add to the periphery of the existing fiber cell mass, displacing older fiber cells toward the interior of the expanding lens. In some cases, the central fiber cells are retained for life.

In some embodiments, lens epithelium comprises lens epithelial stem and progenitor cells (also referred to herein as lens epithelial stem/progenitor cells, lens epithelial stem/progenitor-like cells or LECs). In some cases, LECs proliferate and differentiate into lens fiber cells.

### Cataracts

Cataract is a refractory ocular disease which occurs and develops due to various factors such as long-term ultraviolet exposure, radiation, diabetes, hypertension with the most common cause being age. Most cataracts develop when aging or injury changes the tissue that makes up the eye's lens. Cataracts account for 48% of world blindness or over 18 million people have some cataract development according to the World Health Organization (WHO). The disease subsequently leads to lower vision due to lens opacity. Symptoms of cataract include, but are not limited to, clouded, blurred or dim vision, increasing difficulty with vision at night, sensitivity to light and glare, need for brighter light for reading and other activities, seeing “halos” around lights, frequent changes in eyeglass or contact lens prescription, fading or yellowing of colors, and double vision in a single eye. At first, the cloudiness in the vision caused by a cataract affect only a small part of the eye's lens and causing unawareness of any vision loss. As the cataract grows larger, it clouds more of the lens and distorts the light passing through the lens. This leads to more noticeable symptoms.

Formation of Cataracts

In some embodiments, the lenses in the eyes become less flexible, less transparent and thicker with age. In some cases, age-related and other medical conditions cause tissues within the lens to break down and clump together, clouding small areas within the lens. As the cataract continues to develop, the clouding becomes more dense. A cataract scatters and blocks the light as it passes through the lens, preventing a sharply defined image from reaching the retina. As a result, vision becomes blurred. In some cases, cataracts develop in one or both eyes, and may not develop evenly.

Types of Cataracts

In some embodiments, the types of cataracts comprise partial or complete cataract, stationary or progressive cataract, or hard or soft cataract and can be classified into the following categories.

Nuclear cataracts—The most common type of cataract, involves the central or ‘nuclear’ part of the lens. Nuclear cataract may at first cause more near-sightedness or even a temporary improvement in reading vision. But with time, the lens gradually turns more densely yellow and further clouds the vision. As the cataract slowly progresses, the lens may even turn brown. In advanced stages, it is called brunescent cataract. Advanced yellowing or browning of the lens can lead to difficulty distinguishing between shades of color. This type of cataract can present with a shift to nearsightedness and causes problems with distance vision, while reading is less affected.

Cortical cataracts—Cortical cataracts are cataracts that affect the edges of the lens and are caused due to the lens cortex (outer layer) becoming opaque. They occur when changes in the fluid contained in the periphery of the lens causes fissuring. Cortical cataract begins as whitish, wedge-shaped opacities or streaks on the outer edge of the lens cortex. As it slowly progresses, the streaks extend to the center and interfere with light passing through the center of the lens. Symptoms often include problems with glare and light scatter at night.

Posterior subcapsular cataracts—Posterior subcapsular cataracts are cloudy at the back of the lens adjacent to the capsule (or bag) in which the lens sits. Posterior subcapsular cataract starts as a small, opaque area that usually forms near the back of the lens, right in the path of light. Posterior subcapsular cataract often interferes with reading vision, reduces vision in bright light, and causes glare or halos around lights at night. These types of cataracts tend to progress faster than other types.

Secondary cataracts—Cataracts that form after surgery for other eye problems, such as glaucoma. Cataracts also develop in people who have other health problems, such as diabetes. Cataracts are sometimes linked to steroid use or can also result from being around toxic substances, ultraviolet light, or radiation, or from taking medicines such as corticosteroids or diuretics.

Traumatic cataracts—Cataracts that develop after an eye injury, sometimes years later.

Radiation cataracts—Cataracts that develop after exposure to some types of radiation.

Pediatric Cataracts

In some embodiments, cataract further comprises pediatric cataracts. In children, cataract causes more visual disability than any other form of treatable blindness. Children with untreated, visually significant cataracts face a lifetime of blindness at tremendous quality of life and socioeconomic costs to the child, the family, and the society. More than 200,000 children are blind from unoperated cataract, from complications of cataract surgery, or from ocular anomalies associated with cataracts. Many more children suffer from partial cataracts that may slowly progress over time, increasing the visual difficulties as the child grows. The cumulative risk of cataract during the growing years is as high as 1 per 1000.

Cataracts in children can be classified using a number of methods including age of onset, etiology, and morphology.

Age of Onset:

Congenital or infantile cataracts—Congenital cataracts are cataracts one is born with. Some babies are born with cataracts or develop them in childhood, often in both eyes. These cataracts are genetic, or associated with an intrauterine infection or trauma. These cataracts also may be due to certain conditions, such as myotonic dystrophy, galactosemia, neurofibromatosis type 2 or rubella. Congenital cataracts don't always affect vision, but if they do they're usually removed soon after detection. Congenital cataract, which may be detected in adulthood, has a different classification and includes lamellar, polar, and sutural cataracts. Some morphological categories of cataracts such as anterior polar, central fetal nuclear, and posterior polar clearly indicate a congenital onset, while others such as cortical or lamellar may be associated either with a later onset or be congenital in nature.

Acquired and Juvenile cataracts—Acquired cataract is one from an external cause, as opposed to one in which the cause is genetically determined, such as a mutation in one of the crystalline genes. In some instances, acquired cataract is used to indicate an onset after infancy, which does not necessarily indicate a non-genetic cause. Juvenile cataracts are those with an onset in childhood, after infancy, irrespective of underlying etiology.

Etiology:

Genetic—Approximately 50% of childhood cataracts are caused by mutations in genes that code for proteins involved in lens structure or clarity. Examples of diseases causing congenital or early acquired cataracts includes but are not limited to Hyperferritinemia-cataract syndrome, Coppock-like cataracts, Volkmann type congenital cataract, Zonular with sutural opacities, Posterior polar 1 (CTPP1), Posterior polar 2 (CTPP2), Posterior polar 3 (CTPP3), Posterior pole 4 (CTPP4), Posterior pole 5 (CTPP5), Zonular pulverulent 1 (CZP1), Zonular pulverulent 3 (CZP3), Anterior polar cataract 1, Anterior polar cataract 2, Cerulean type 1 (CCA1), Cerulean type 2 (CCA2), Cerulean type 3 (CCA3), Crystalline aculeiform cataract, Nonnuclear polymorphic congenital cataract, Sutural cataract with punctate and cerulean opacities, Myotonic dystrophy 1 (DM1), Polymorphic and lamellar cataracts, Cataract, autosomal dominant, multiple types 1, Congenital cataracts, facial dysmorphism, and neuropathy (CCFDN), Marinesco-Sjögren syndrome, Warburg micro syndrome 1, Warburg micro syndrome 1, Warburg micro syndrome 2, Warburg micro syndrome 3, Martsolf syndrome, Hallermann-Streiff syndrome (Francois dyscephalic syndrome, Rothmund-Thomson syndrome, Smith-Lemli-Opitz syndrome, Congenital nuclear cataracts 2, Norrie disease, and Nance Horan syndrome.

Secondary

(a) Uveitis—Cataracts develop in patients with uveitis as a result of the chronic ocular inflammation or secondary to the chronic use of steroids. Surgery for such cataracts is complicated by severe postoperative inflammation. Many patients have a pupillary membrane that covers the lens and attaches to the iris. Such membranes are peeled off of the anterior lens capsule at the time of surgery to facilitate lens removal.

Juvenile idiopathic arthritis: One of the more common causes of anterior uveitis in children. The use of systemic antimetabolites has led to better control of uveitis in such patients and to a reduction in the incidence of cataracts.

Other types of uveitis can also cause cataracts either because of the inflammation or as a complication of steroid use.

(b) Intraocular tumors—It is very uncommon for cataracts to develop as a consequence of intraocular tumors. The lens is characteristically clear in patients with untreated retinoblastoma. Treatments of the tumor such as radiotherapy sometimes lead to the development of cataracts, in which case timing of cataract removal has to be very carefully considered and surgery only performed when all tumors in the eye has been eradicated. Patients with radiation cataracts can have significant ocular surface dryness and will not tolerate contact lenses.

(c) Chronic retinal detachment—These cataracts are seen in the setting of injuries or in association with Stickler syndrome. If the lens is totally opaque, preoperative ultrasonography is performed to rule out a chronic retinal detachment. The presence of an afferent pupillary defect is a poor prognostic sign.

(d) Maternal infection (rubella)—This type of cataract is not seen in countries where rubella has been eradicated, but continues to occur in some parts of the world.

Iatrogenic

(a) Radiation—External beam radiation is avoided in patients with retinoblastoma. The eye is typically shielded if radiation is given to the brain or other parts of the head and neck.

(b) Systemic steroids are very rare causes of cataracts in children. The typical steroid-induced cataract is posterior subcapsular.

(c) Vitrectomy—A large percentage of children who undergo vitrectomy develop cataracts. These are mostly posterior subcapsular.

(d) Laser for retinopathy of prematurity—Cataracts can develop from thermal injury to the lens when a prominent tunica vasculosa lentis is present.

Morphology:

Diffuse/Total—This is not an uncommon type of congenital cataract. There are no specific causes of diffuse or total cataracts.

Anterior

(a) Anterior polar—The opacity is in the capsule itself and can protrude into the anterior chamber as a small mammillation. There may be an underlying circular layer of cortical opacity slightly larger than the white polar opacity. While the majority are stable and do not interfere with vision, some can progress and require surgical removal. They can be dominantly inherited, especially in bilateral cases. Unilateral cases can be associated with anisometropia (astigmatism or hyperopia), which if left untreated can cause amblyopia, even if the cataract itself is not visually significant.

(b) Pyramidal—These are usually larger than polar cataracts and more likely to progress to visual significance. They are difficult to remove with a vitrectomy instrument and may require excision and removal with forceps before the rest of the lens is aspirated.

(c) Anterior lenticonus—This refers to a thinned-out central anterior capsule with or without anterior cortical opacities. Anterior lenticonus is said to be characteristic of Alport syndrome. Spontaneous rupture of the lens can occur, resulting in a hydrated total cataract.

Cortical Lamellar

In this type of cataract, the opacification is of a lamella (an ovoid layer of cortex) that can be visualized between adjacent clear lamellae. These are frequently associated with radial “rider” opacities. Familial lamellar cataracts are mostly autosomal dominant and are generally associated with a good visual prognosis after their removal. They can be stable or may be associated with progressive opacification of intervening cortex, necessitating removal.

Fetal Nuclear

These opacities occupy the central-most part of the lens. They can be dot-like or can be quite dense. They generally measure 2-3.5 mm and can be associated with microphthalmia. They are said to be associated with a higher incidence of postoperative glaucoma because of associated microphthalmia and the need for surgery early in infancy.

Posterior Polar

In this type of cataract, the opacity is in the capsule itself. It is necessary to differentiate posterior polar from posterior subcapsular cataracts. Posterior polar cataracts are genetically determined and some have been associated with mutations in PITX3.

Posterior Lentiglobus (Lenticonus)

In this group of conditions, the central and sometimes paracentral posterior capsule is thin and bulges posteriorly. This usually occurs at the location where the hyaloid system attaches to the eye. The distortion can cause a localized area of extreme myopic refraction. There may or may not be subcapsular cortical opacification. Interference with vision can be the result of optical distortion or of capsular opacification. Most cases are unilateral, although bilateral and familial cases have been reported. Surgery is associated with good visual outcomes in most cases. Spontaneous rupture of the lens can rarely occur, leading to abrupt progression to total cataract.

Posterior Subcapsular

These can be congenital but are more commonly acquired as a result of injury or steroid use. The opacities are cortical and do not involve the capsule proper.

Persistent Fetal Vasculature (PFV) (Severe Varieties are Still Referred to as Persistent Hyperplastic Primary Vitreous)

The lens opacities in patients with PFV are generally capsular and can be associated with shrinkage, thickening, and vascularization of the capsule. There may be a posterior plaque outside or involving the lens capsule with a clear lens that nonetheless must be treated as a cataract.

Traumatic Disruption of Lens

In children, traumatic anterior lens capsule rupture quickly results in a hydrated white cataract. However, in children, lens cortex in the anterior chamber may be well tolerated without an intraocular pressure (TOP) rise. Cataract surgery can often be delayed for a few days or up to 3 or 4 weeks to allow the traumatic iritis to subside before the cataract and IOL surgery.

### Methods of Use

In some embodiments, disclosed herein is use of a biomaterial composition to maintain the structural integrity of a lens anterior capsule of an eye of a subject and to induce expansion of lens epithelial stem and progenitor cells in situ, wherein the biomaterial composition is administered into the lens anterior capsule through an capsulorhexis opening located at a peripheral area of the lens anterior capsule, and wherein the contents of the lens is removed prior to administration of the biomaterial composition.

In some embodiments, a biomaterial composition described herein are utilized to promote expansion of LECs. In some cases, a biomaterial composition described herein are utilized to promote or facilitate proliferation and differentiation of LECs into lens fiber cells. In some instances, a biomaterial composition comprises human serum and a fibroblast growth factor (FGF). In some cases, the fibroblast growth factor is a human fibroblast growth factor.

In some embodiments, a biomaterial composition optionally comprises one or more nutrients, additives, or a combination thereof. In some cases, the one or more nutrients, additives or a combination thereof promote cell proliferation, cell differentiation or cell viability. In some cases, one or more nutrients comprise a composition of amino acids. In some cases, the composition of amino acids comprises one or more amino acids selected from: alanine, arginine, asparagine, aspartic acid, cysteine, glutamic acid, glutamine, glycine, proline, serine, and tyrosine. In some cases, the composition of amino acids comprises one or more amino acids selected from: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine. In some cases, the composition of amino acids comprise one or more amino acids selected from: alanine, asparagine, aspartic acid, glutamic acid, glycine, proline and serine. In some cases, the composition of amino acids comprises alanine, arginine, asparagine, aspartic acid, cysteine, glutamic acid, glutamine, glycine, proline, serine, and tyrosine. In some cases, the composition of amino acids comprises alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine. In some cases, the composition of amino acids comprises alanine, asparagine, aspartic acid, glutamic acid, glycine, proline and serine.

In some embodiments, one or more nutrients comprise a glucose source. In some cases, a biomaterial composition comprises a glucose source.

In some embodiments, one or more nutrient comprises a pyruvate. In some cases, a biomaterial composition comprises a pyruvate.

In some embodiments, one or more nutrient comprise at least one vitamin. Exemplary vitamins include, but are not limited to, folic acid, nicotinamide, riboflavin, B₁₂, choline chloride, myo-inositol, niacinamide, D-Pantothenic acid, Pyridoxal-HCl, thiamine-HCl, and the like. In some cases, one or more nutrient comprise at least one vitamin selected from folic acid, nicotinamide, riboflavin, B₁₂, choline chloride, myo-inositol, niacinamide, D-Pantothenic acid, Pyridoxal-HCl, and thiamine-HCl. In some cases, a biomaterial composition comprises at least one vitamin selected from folic acid, nicotinamide, riboflavin, B₁₂, choline chloride, myo-inositol, niacinamide, D-Pantothenic acid, Pyridoxal-HCl, and thiamine-HCl.

In some embodiments, an additive comprises an inorganic salt. Exemplary inorganic salts include, but are not limited to, calcium chloride, potassium chloride, magnesium sulfate, sodium chloride, monosodium phosphate, potassium phosphate, sodium bicarbonate, and sodium phosphate. In some instances, an additive comprises calcium chloride, potassium chloride, magnesium sulfate, sodium chloride, monosodium phosphate, potassium phosphate, sodium bicarbonate, sodium phosphate, or a combination thereof. In some cases, a biomaterial composition comprises calcium chloride, potassium chloride, magnesium sulfate, sodium chloride, monosodium phosphate, potassium phosphate, sodium bicarbonate, sodium phosphate, or a combination thereof.

In some instances, a biomaterial composition in a range of about 0.1× to about 10× concentration is utilized with a method described herein. In some cases, a concentration range of about 0.1× to about 9×, about 0.5× to about 8×, about 0.5× to about 7×, about 0.5× to about 6×, about 0.5× to about 5×, about 0.5× to about 4×, about 0.5× to about 3×, about 0.5× to about 2×, about 0.5× to about 1×, about 1× to about 10×, about 1× to about 9×, about 1× to about 8×, about 1× to about 7×, about 1× to about 6×, about 1× to about 5×, about 1× to about 4×, about 1× to about 3×, about 1× to about 2×, about 2× to about 10×, about 2× to about 9×, about 2× to about 8×, about 2× to about 7×, about 2× to about 6×, about 2× to about 5×, about 2× to about 4×, about 2× to about 3×, about 4× to about 10×, about 4× to about 9×, about 4× to about 8×, about 4× to about 7×, about 4× to about 6×, about 4× to about 5×, about 5× to about 10×, about 5× to about 9×, about 5× to about 8×, about 5× to about 7× or about 5× to about 6× concentration is utilized with a method described herein.

In some instances, a biomaterial composition in a range of about 0.1× to about 10× concentration is utilized for maintaining structural integrity and to induce expansion of lens epithelial stem and progenitor cells in situ. In some cases, a concentration range of about 0.1× to about 9×, about 0.5× to about 8×, about 0.5× to about 7×, about 0.5× to about 6×, about 0.5× to about 5×, about 0.5× to about 4×, about 0.5× to about 3×, about 0.5× to about 2×, about 0.5× to about 1×, about 1× to about 10×, about 1× to about 9×, about 1× to about 8×, about 1× to about 7×, about 1× to about 6×, about 1× to about 5×, about 1× to about 4×, about 1× to about 3×, about 1× to about 2×, about 2× to about 10×, about 2× to about 9×, about 2× to about 8×, about 2× to about 7×, about 2× to about 6×, about 2× to about 5×, about 2× to about 4×, about 2× to about 3×, about 4× to about 10×, about 4× to about 9×, about 4× to about 8×, about 4× to about 7×, about 4× to about 6×, about 4× to about 5×, about 5× to about 10×, about 5× to about 9×, about 5× to about 8×, about 5× to about 7× or about 5× to about 6× concentration is utilized for maintaining structural integrity and to induce expansion of lens epithelial stem and progenitor cells in situ.

In some cases, a concentration of about 0.1×, 0.2×, 0.3×, 0.4×, 0.5×, 0.6×, 0.7×, 0.8×, 0.9×, 1×, 2×, 3×, 4×, 5×, 6×, 7×, 8×, 9× or 10× is utilized with a method described herein. In some instances, a concentration of about 0.1× is utilized. In some instances, a concentration of about 0.2× is utilized. In some instances, a concentration of about 0.3× is utilized. In some instances, a concentration of about 0.4× is utilized. In some instances, a concentration of about 0.5× is utilized. In some instances, a concentration of about 0.6× is utilized. In some instances, a concentration of about 0.7× is utilized. In some instances, a concentration of about 0.8× is utilized. In some instances, a concentration of about 0.9× is utilized. In some instances, a concentration of about 1× is utilized. In some instances, a concentration of about 2× is utilized. In some instances, a concentration of about 3× is utilized. In some instances, a concentration of about 4× is utilized. In some instances, a concentration of about 5× is utilized. In some instances, a concentration of about 6× is utilized. In some instances, a concentration of about 7× is utilized. In some instances, a concentration of about 8× is utilized. In some instances, a concentration of about 9× is utilized. In some instances, a concentration of about 10× is utilized. In some cases, a concentration of about 0.1×, 0.2×, 0.3×, 0.4×, 0.5×, 0.6×, 0.7×, 0.8×, 0.9×, 1×, 2×, 3×, 4×, 5×, 6×, 7×, 8×, 9× or 10× is utilized for maintaining structural integrity and to induce expansion of lens epithelial stem and progenitor cells in situ.

In some embodiments, a biomaterial composition described herein is administered to the lens anterior capsule in a volume sufficient to replace the volume lost due to the removal of the content of the lens from the lens anterior capsule. In some cases, the biomaterial composition is administered to the lens anterior capsule in a volume sufficient to maintain the structural integrity of the lens anterior capsule. In some instances, the volume is about 10 μL to about 300 μL. In some instances, the volume is about 10 μL to about 250 μL. In some instances, the volume is about 10 μL to about 200 μL. In some instances, the volume is about 50 μL to about 300 μL. In some instances, the volume is about 50 μL to about 250 μL. In some instances, the volume is about 50 μL to about 200 μL. In some instances, the volume is about 50 μL to about 100 μL. In some instances, the volume is about 100 μL to about 300 μL. In some instances, the volume is about 100 μL to about 250 μL. In some instances, the volume is at least 10 μL. In some instances, the volume is at least 50 μL. In some instances, the volume is at least 100 μL. In some instances, the volume is at least 150 μL. In some instances, the volume is at least 200 μL. In some instances, the volume is at least 250 μL. In some instances, the volume is at least 300 μL. In some instances, the volume is at most 10 μL. In some instances, the volume is at most 50 μL. In some instances, the volume is at most 100 μL. In some instances, the volume is at most 150 μL. In some instances, the volume is at most 200 μL. In some instances, the volume is at most 250 μL. In some instances, the volume is at most 300 μL.

In some embodiments, the capsulorhexis opening is about 1.0 to 2.0 mm in diameter. In some cases, the capsulorhexis opening is about 1.0 to 1.5 mm in diameter. In some instances, the capsulorhexis opening is about 1 mm in diameter, about 1.1 mm in diameter, about 1.2 mm in diameter, about 1.3 mm in diameter, about 1.4 mm in diameter, about 1.5 mm in diameter, about 1.6 mm in diameter, about 1.7 mm in diameter, about 1.8 mm in diameter, about 1.9 mm in diameter, or about 2 mm in diameter. In some cases, the capsulorhexis opening is about 1 mm in diameter. In some cases, the capsulorhexis opening is about 1.1 mm in diameter. In some cases, the capsulorhexis opening is about 1.2 mm in diameter. In some cases, the capsulorhexis opening is about 1.3 mm in diameter. In some cases, the capsulorhexis opening is about 1.4 mm in diameter. In some cases, the capsulorhexis opening is about 1.5 mm in diameter. In some cases, the capsulorhexis opening is about 1.6 mm in diameter. In some cases, the capsulorhexis opening is about 1.7 mm in diameter. In some cases, the capsulorhexis opening is about 1.8 mm in diameter. In some cases, the capsulorhexis opening is about 1.9 mm in diameter. In some cases, the capsulorhexis opening is about 2 mm in diameter.

In some embodiments, the capsulorhexis opening is less than 1.0 mm to less than 2.0 mm in diameter. In some instances, the capsulorhexis opening is less than 1.0 mm in diameter. In some instances, the capsulorhexis opening is less than 1.1 mm in diameter. In some instances, the capsulorhexis opening is less than 1.2 mm in diameter. In some instances, the capsulorhexis opening is less than 1.3 mm in diameter. In some instances, the capsulorhexis opening is less than 1.4 mm in diameter. In some instances, the capsulorhexis opening is less than 1.5 mm in diameter. In some instances, the capsulorhexis opening is less than 1.6 mm in diameter. In some instances, the capsulorhexis opening is less than 1.7 mm in diameter. In some instances, the capsulorhexis opening is less than 1.8 mm in diameter. In some instances, the capsulorhexis opening is less than 1.9 mm in diameter. In some instances, the capsulorhexis opening is less than 2 mm in diameter.

In some instances, the capsulorhexis opening is located away from the central visual axis of the eye. In such cases, the incision minimizes visual impairment due to improper healing of the incision.

In some embodiments, the use of a biomaterial composition to maintain the structural integrity of a lens anterior capsule and to induce expansion of lens epithelial stem and progenitor cells in situ occurs in the eye of a subject having cataract. In some cases, the subject is a human. In some instances, the subject is a human aged 18 or older. In other instances, the subject is a human aged 17 or younger. In some cases, the subject is an adult human. In other cases, the subject is a child or an infant.

In some embodiments, the use of a biomaterial composition to maintain the structural integrity of a lens anterior capsule and to induce expansion of lens epithelial stem and progenitor cells in situ occurs in the eye of a subject having a pediatric cataract.

In some embodiments, the use of a biomaterial composition to maintain the structural integrity of a lens anterior capsule and to induce expansion of lens epithelial stem and progenitor cells in situ occurs in the eye of a subject having congenital cataract (or infantile cataract).

In some embodiments, the use of a biomaterial composition to maintain the structural integrity of a lens anterior capsule and to induce expansion of lens epithelial stem and progenitor cells in situ occurs in the eye of a subject having acquired and juvenile cataract.

In some embodiments, the use of a biomaterial composition to maintain the structural integrity of a lens anterior capsule and to induce expansion of lens epithelial stem and progenitor cells in situ occurs in the eye of a subject having cataract selected from nuclear cataract, cortical cataract, posterior subcapsular cataract, secondary cataract, traumatic cataract, and radiation cataract. In some instances, the use of a biomaterial composition to maintain the structural integrity of a lens anterior capsule and to induce expansion of lens epithelial stem and progenitor cells in situ occurs in the eye of a subject having nuclear cataract. In some instances, the use of a biomaterial composition to maintain the structural integrity of a lens anterior capsule and to induce expansion of lens epithelial stem and progenitor cells in situ occurs in the eye of a subject having cortical cataract. In some instances, the use of a biomaterial composition to maintain the structural integrity of a lens anterior capsule and to induce expansion of lens epithelial stem and progenitor cells in situ occurs in the eye of a subject having posterior subcapsular cataract. In some instances, the use of a biomaterial composition to maintain the structural integrity of a lens anterior capsule and to induce expansion of lens epithelial stem and progenitor cells in situ occurs in the eye of a subject having secondary cataract. In some instances, the use of a biomaterial composition to maintain the structural integrity of a lens anterior capsule and to induce expansion of lens epithelial stem and progenitor cells in situ occurs in the eye of a subject having traumatic cataract. In some instances, the use of a biomaterial composition to maintain the structural integrity of a lens anterior capsule and to induce expansion of lens epithelial stem and progenitor cells in situ occurs in the eye of a subject having radiation cataract.

In some embodiments, after making a capsulorhexis opening in the lens anterior capsule, the contents of the lens is removed, including, e.g., cataract and optionally native lens. In some cases, endogenous lens epithelial stem and progenitor cells (LECs) are preserved in the lens anterior capsule.

In some cases, lens epithelial stem and progenitor cells express Pax6 and/or Bmi-1. In some cases, the LECs expressing Pax6 and/or Bmi-1 expand or proliferate and subsequently differentiate into lens fiber cells.

In some instances, use and methods described herein do not involve an implantation of an artificial intraocular lens (IOL).

In some cases, use and methods described herein results in reduced visual axis opacification (VAO) relative to a method comprising a capsulorhexis procedure comprising central capsulorhexis opening and implantation of an artificial intraocular lens.

In additional cases, use and methods described herein results in lowered incidents of complications selected from the group consisting of corneal edema, anterior chamber inflammation, and visual axis opacification.

### System and Devices for Lens Regeneration

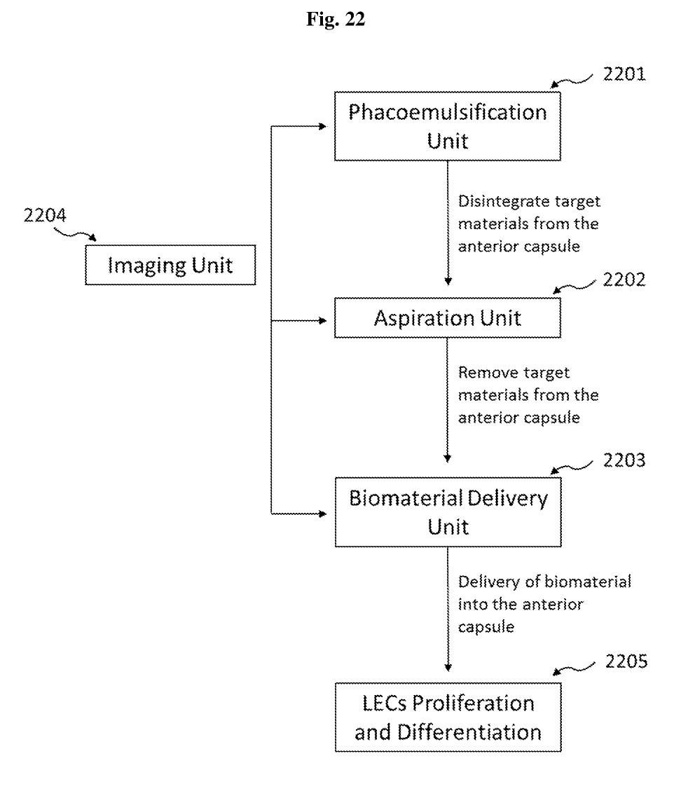

In certain embodiments, disclosed herein relates to a system and methods thereof for treating cataracts. In some instances, the system includes a phacoemulsification unit, an aspiration unit, and a biomaterial delivery unit, optionally a detector and a computer/comparator. In some cases, the system includes an imaging unit (e.g., a detector) (2204) for visualizing at one or more steps during phacoemulsification, aspiration or delivery of a biomaterial composition; a phacoemulsification unit (2201) for disintegration of target materials (e.g., cataract) from the anterior capsule; an aspiration unit (2202) for removing the target materials from the anterior capsule; and a biomaterial delivery unit (2203) to deliver biomaterial compositions to facilitate LECs proliferation and differentiation (2205), in which all of the units are operationally connected to a computer (see FIG. 22). In some instances, in the system the detector is operationally connected to the computer/comparator, and the computer/comparator is connected directly to the phacoemulsification unit (2201), the aspiration unit (2202) and the biomaterial delivery unit (2203). With this combination, the system is used to generate and direct a phacoemulsification unit or tool toward an eye for an ophthalmic surgical procedure as envisioned for the present invention. In some instances, the system may comprise at least one of the following to facilitate removal of cataract material from a sample lens capsule: lasers, optical coherence tomography (OCT) sensors, imaging systems, video systems, location sensors, flush devices, aspiration devices, and robotic articulation control.

In some instances, phacoemulsification (Phaco) is a technique used to extract the cataract material and maintain the integrity of the anterior chamber and lens capsule. The term “phacoemulsification” as used herein refers to ultrasound and laser-based emulsification procedures, as well as combinations of ultrasound and laser procedures, used to disintegrate target interior eye tissue, typically the lens, for cataract surgery.

In some embodiments, the phacoemulsification unit (2201) involves the use of a machine with an ultrasound and/or laser hand piece equipped with a tip to emulsify the lens of the patient. In some embodiments, the tip is a narrow or thin probe that can be designed to be inserted into the peripheral area of the lens, instead of the central axis area, to preserve a nearly intact transparent lens capsule and layer of lens epithelial stem or progenitor cells, which have regenerative potential and are critically required for the regeneration of a natural lens. In some embodiments, the phacoemulsification tool or probe is about 3 mm or less, such as 2 mm and 1 mm. In some embodiments, the phacoemulsification is less than about 1 mm. In some embodiments, the tip is made of titanium or steel or other material that vibrates at ultrasonic frequency and the lens material is emulsified. In other embodiments, the phacoemulsification tip is a laser capable of generating a so-called “femtosecond” laser beam.

### Laser Unit

In some embodiments, the generated laser beam includes a sequence of laser pulses having a very ultra-short duration (e.g. less than approximately 500 fs). In some instances, the laser unit includes a beam steering component for moving the focal spot of the laser along a selected path to emulsify a volume of target tissue. In some embodiments, the laser signal and energy is conveyed to the tip of the tool via a photonic waveguide, a set of mirrors, or a set of mirrors and lenses. Importantly, the laser beam must be capable of performing Laser Induced Optical Breakdown (LIOB) on selected target tissue inside the eye. Further, it is important for there to be a precise performance of this LIOB. Such precision requires there be a capability of imaging the target tissue that is to be altered by LIOB. In some embodiments, tool is able to break up the cataract with the laser in small precise regions due to the strong absorption of the laser light by the cataract material, or water, mesh, or any thermal or mechanical effect. In some embodiments, the laser light in tool is altered to “undercut” the larger pieces of cataract material, i.e., use small cuts to remove large pieces. In some embodiments, the pulse energy, repetition rate, and pulse duration of the laser in tool is controlled in real-time. In controlling these parameters, a user of tool alters the extent and speed of cataract material removal. In some instances, the laser applies a number of pulses to the lens in a pre-designed pattern to remove the lens matter. In some instances, the tip of the tool is shaped to provide maximum cutting effect. In some instances, the shapes of the laser tool tip is flat, round, tapered to a point, or a combination of the flat, round and tapered shapes.

### Flush and Aspiration Tools (2202)

In some embodiments, the invention includes a flush and aspiration tool (e.g., an aspiration unit, 2202) to remove debris from the capsule. In some embodiments, the flush and aspiration capabilities are conjoined on the same tool as the emulsification tool (e.g., the phacoemulsification unit, 2201). In the alternative, a second tool includes a dedicated aspiration and flush tool. In general, aspiration unit (2202) comprises a power source that provides electricity to a vacuum pump coupled through a hose to a dampener. In some instances, the aspiration flow is transferred from the dampener to a tool through a tube. The dampener (e.g., represented by a plunger within a cylinder) moderates spikes and dips in the aspiration pressure in cases of air blockage or occlusion in tool, for example, through a flow rate meter. In some instances, the size of the flush and aspiration channels directly influences the size of the pieces of cataract that are extracted from the lens capsule.

In some embodiments, the invention incorporates a pump controlled by a computer, a pressure vessel, and a flow rate meter. In apparatus, fluid is supplied to pump. Pump directionally forwards the fluid to pressure vessel via a tube. The fluid pumped into pressure vessel forces liquid that was originally resting in pressure vessel toward the flow rate meter. The flow rate meter detects the velocity of the liquid prior to output from apparatus. Information regarding the velocity of liquid flow is sent to computer, which can send pressure signal to pump. This creates a feedback loop; by communicating a flow rate feedback signal to computer, computer can respond to the velocity of the exiting liquid by controlling the pump through pressure signal. Hence, if the flow of liquid is too great, the flow of fluid from the pump through tube is adjusted downward. Alternatively, if the flow of liquid is too low, the flow of fluid from pump through tube is increased to exert more pressure on the liquid in pressure vessel. The increased pressure in pressure vessel results in increased flow of liquid. This feedback loop enables apparatus to moderate its liquid flow to a desired flush rate at output. In some instances, the disclosure includes a throttle valve receiving the flow control signal from either a computer, central processing unit, microcontroller, ASIC, or other control circuitry. In some instances, the throttle valve further affects fluid flow by limiting (“throttling”) the fluid output from apparatus.

During the emulsification and aspiration of the lens cataract material, certain portions of the capsule which carry stem cells (such as the anterior and/or posterior portions) are at risk of being damaged from the emulsification unit and the aspiration force. For example, if portions of the lens capsule membrane are sucked into the aspiration tube, the anterior and/or posterior portion may be stressed and torn. This increases the risk of intrusion of the vitreous fluid into the anterior portion of the eye, which cause infections and other eye diseases. To minimize that possibility, in some embodiments, the emulsification unit is extended beyond the end of the suction tube to act as a probe when the emulsifier is turned off. The use of the fiber as a probe prevents the suction tube from approaching the capsular membrane and damaging it. The shape of the probe is optimized to minimize damage to the membrane. Examples of the shaped tips include rounded or circular tips. In some embodiments, the tip have flush capabilities and aspiration capabilities to extract the cataract material and maintain the integrity of the anterior chamber and lens capsule.

In some embodiments, the lens capsule remain intact, where bilateral incisions are made for phacoemulsification tips, and for aspirating tips and/or irrigating tips for removing the bulk of the lens, Thereafter, the complete contents of the lens capsule are successfully rinsed/washed, which expels the debris that lead to secondary cataracts. Then, with the lens capsule intact, a minimal incision is made for a biomaterial delivery unit (2203) to inject or deliver biomaterial through incision to fill the capsule.

In some embodiments, lens stem and progenitor cell regeneration is enhanced by the delivery of biomaterial to the lens capsule. As such, several embodiments vary the components of the delivered biomaterial to affect an optimal regeneration of lens stem and progenitor cells. For example, the pump is used to biomaterial fluid in addition to or in lieu of aspiration fluid, or alternatively a syringe is used to introduce biomaterial.

### Biomaterials for Enhancing Lens Stem/Progenitor Cell Growth in Situ

In some embodiments, a biomaterial composition described herein are utilized to promote expansion of LECs. In some cases, a biomaterial composition described herein are utilized to promote or facilitate proliferation and differentiation of LECs into lens fiber cells. In some instances, a biomaterial composition comprises human serum and a fibroblast growth factor (FGF). In some cases, the fibroblast growth factor is a human fibroblast growth factor.

In some embodiments, a biomaterial composition optionally comprises one or more nutrients, additives, or a combination thereof. In some cases, the one or more nutrients, additives or a combination thereof promote cell proliferation, cell differentiation or cell viability. In some cases, one or more nutrients comprise a composition of amino acids. In some cases, the composition of amino acids comprises one or more amino acids selected from: alanine, arginine, asparagine, aspartic acid, cysteine, glutamic acid, glutamine, glycine, proline, serine, and tyrosine. In some cases, the composition of amino acids comprises one or more amino acids selected from: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine. In some cases, the composition of amino acids comprises one or more amino acids selected from: alanine, asparagine, aspartic acid, glutamic acid, glycine, proline and serine. In some cases, the composition of amino acids comprises alanine, arginine, asparagine, aspartic acid, cysteine, glutamic acid, glutamine, glycine, proline, serine, and tyrosine. In some cases, the composition of amino acids comprises alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine. In some cases, the composition of amino acids comprises alanine, asparagine, aspartic acid, glutamic acid, glycine, proline and serine.

In some embodiments, one or more nutrients comprise a glucose source. In some cases, a biomaterial composition comprises a glucose source.

In some embodiments, one or more nutrient comprises a pyruvate. In some cases, a biomaterial composition comprises a pyruvate.

In some embodiments, one or more nutrient comprise at least one vitamin. Exemplary vitamins include, but are not limited to, folic acid, nicotinamide, riboflavin, B₁₂, choline chloride, myo-inositol, niacinamide, D-Pantothenic acid, Pyridoxal-HCl, thiamine-HCl, and the like. In some cases, one or more nutrient comprise at least one vitamin selected from folic acid, nicotinamide, riboflavin, B₁₂, choline chloride, myo-inositol, niacinamide, D-Pantothenic acid, Pyridoxal-HCl, and thiamine-HCl. In some cases, a biomaterial composition comprises at least one vitamin selected from folic acid, nicotinamide, riboflavin, B₁₂, choline chloride, myo-inositol, niacinamide, D-Pantothenic acid, Pyridoxal-HCl, and thiamine-HCl.

In some embodiments, an additive comprises an inorganic salt. Exemplary inorganic salts include, but are not limited to, calcium chloride, potassium chloride, magnesium sulfate, sodium chloride, monosodium phosphate, potassium phosphate, sodium bicarbonate, and sodium phosphate. In some instances, an additive comprises calcium chloride, potassium chloride, magnesium sulfate, sodium chloride, monosodium phosphate, potassium phosphate, sodium bicarbonate, sodium phosphate, or a combination thereof. In some cases, a biomaterial composition comprises calcium chloride, potassium chloride, magnesium sulfate, sodium chloride, monosodium phosphate, potassium phosphate, sodium bicarbonate, sodium phosphate, or a combination thereof.

In some instances, a biomaterial composition in a range of about 0.1× to about 10× concentration is utilized with a system described herein. In some cases, a concentration range of about 0.1× to about 9×, about 0.5× to about 8×, about 0.5× to about 7×, about 0.5× to about 6×, about 0.5× to about 5×, about 0.5× to about 4×, about 0.5× to about 3×, about 0.5× to about 2×, about 0.5× to about 1×, about 1× to about 10×, about 1× to about 9×, about 1× to about 8×, about 1× to about 7×, about 1× to about 6×, about 1× to about 5×, about 1× to about 4×, about 1× to about 3×, about 1× to about 2×, about 2× to about 10×, about 2× to about 9×, about 2× to about 8×, about 2× to about 7×, about 2× to about 6×, about 2× to about 5×, about 2× to about 4×, about 2× to about 3×, about 4× to about 10×, about 4× to about 9×, about 4× to about 8×, about 4× to about 7×, about 4× to about 6×, about 4× to about 5×, about 5× to about 10×, about 5× to about 9×, about 5× to about 8×, about 5× to about 7× or about 5× to about 6× concentration is utilized with a system described herein.

In some instances, a biomaterial composition in a range of about 0.1× to about 10× concentration is utilized for maintaining structural integrity and to induce expansion of lens epithelial stem and progenitor cells in situ. In some cases, a concentration range of about 0.1× to about 9×, about 0.5× to about 8×, about 0.5× to about 7×, about 0.5× to about 6×, about 0.5× to about 5×, about 0.5× to about 4×, about 0.5× to about 3×, about 0.5× to about 2×, about 0.5× to about 1×, about 1× to about 10×, about 1× to about 9×, about 1× to about 8×, about 1× to about 7×, about 1× to about 6×, about 1× to about 5×, about 1× to about 4×, about 1× to about 3×, about 1× to about 2×, about 2× to about 10×, about 2× to about 9×, about 2× to about 8×, about 2× to about 7×, about 2× to about 6×, about 2× to about 5×, about 2× to about 4×, about 2× to about 3×, about 4× to about 10×, about 4× to about 9×, about 4× to about 8×, about 4× to about 7×, about 4× to about 6×, about 4× to about 5×, about 5× to about 10×, about 5× to about 9×, about 5× to about 8×, about 5× to about 7× or about 5× to about 6× concentration is utilized for maintaining structural integrity and to induce expansion of lens epithelial stem and progenitor cells in situ.

In some cases, a concentration of about 0.1×, 0.2×, 0.3×, 0.4×, 0.5×, 0.6×, 0.7×, 0.8×, 0.9×, 1×, 2×, 3×, 4×, 5×, 6×, 7×, 8×, 9× or 10× is utilized with a system described herein. In some instances, a concentration of about 0.1× is utilized. In some instances, a concentration of about 0.2× is utilized. In some instances, a concentration of about 0.3× is utilized. In some instances, a concentration of about 0.4× is utilized. In some instances, a concentration of about 0.5× is utilized. In some instances, a concentration of about 0.6× is utilized. In some instances, a concentration of about 0.7× is utilized. In some instances, a concentration of about 0.8× is utilized. In some instances, a concentration of about 0.9× is utilized. In some instances, a concentration of about 1× is utilized. In some instances, a concentration of about 2× is utilized. In some instances, a concentration of about 3× is utilized. In some instances, a concentration of about 4× is utilized. In some instances, a concentration of about 5× is utilized. In some instances, a concentration of about 6× is utilized. In some instances, a concentration of about 7× is utilized. In some instances, a concentration of about 8× is utilized. In some instances, a concentration of about 9× is utilized. In some instances, a concentration of about 10× is utilized. In some cases, a concentration of about 0.1×, 0.2×, 0.3×, 0.4×, 0.5×, 0.6×, 0.7×, 0.8×, 0.9×, 1×, 2×, 3×, 4×, 5×, 6×, 7×, 8×, 9× or 10× is utilized for maintaining structural integrity and to induce expansion of lens epithelial stem and progenitor cells in situ.

In some embodiments, a biomaterial composition described herein is administered to the lens anterior capsule in a volume sufficient to replace the volume lost due to the removal of the content of the lens from the lens anterior capsule. In some cases, the biomaterial composition is administered to the lens anterior capsule in a volume sufficient to maintain the structural integrity of the lens anterior capsule. In some instances, the volume is about 10 μL to about 300 μL. In some instances, the volume is about 10 μL to about 250 μL. In some instances, the volume is about 10 μL to about 200 μL. In some instances, the volume is about 50 μL to about 300 μL. In some instances, the volume is about 50 μL to about 250 μL. In some instances, the volume is about 50 μL to about 200 μL. In some instances, the volume is about 50 μL to about 100 μL. In some instances, the volume is about 100 μL to about 300 μL. In some instances, the volume is about 100 μL to about 250 μL. In some instances, the volume is at least 10 μL. In some instances, the volume is at least 50 μL. In some instances, the volume is at least 100 μL. In some instances, the volume is at least 150 μL. In some instances, the volume is at least 200 μL. In some instances, the volume is at least 250 μL. In some instances, the volume is at least 300 μL. In some instances, the volume is at most 10 μL. In some instances, the volume is at most 50 μL. In some instances, the volume is at most 100 μL. In some instances, the volume is at most 150 μL. In some instances, the volume is at most 200 μL. In some instances, the volume is at most 250 μL. In some instances, the volume is at most 300 μL.

imaging Sensors

Imaging techniques and sensors which comprise an imaging unit described herein (2204) are used to optimize laser, flush, and aspiration parameters. For example, if it is detected that the tool tip is too close to anatomical structures, the laser power is reduced to reduce the chance of injury. Similarly, flush and aspiration pressure is manipulated to facilitate removal of the cataract material.

In some embodiments, a locational sensor or imaging technique is used to localize different portions of the cataract and the size of the cataract. Such locational sensors or imaging techniques include, but are not limited to, 3D imaging, OCT, MRI, CT, Ultrasound, Intra-operative (OCT) or video systems with processing. In some embodiments, the tool itself has an OCT device. In some embodiments, the tool has multiple degree of freedom (dot) sensors, such as electromagnetic or fiber sensors. Accurate images using the image components described herein is used to define non-treatment safety zones to protect the lens, posterior lens capsule, retina, etc.

In some embodiments, the detector is a type of imaging unit that operates using Optical Coherence Tomography (OCT) techniques. Alternatively, or in addition to the OCT device, the detector includes a Scheimpflug device, confocal imaging device, optical range-finding device, ultrasound device and/or two-photon imaging device. Thus, the detector will include a light source to generate an imaging beam and optics to direct the imaging beam toward the eye. In some instances, these optics include some or all of the optics in the beam steering component of the laser unit. For the system, the imaging beam is used to create three dimensional images of selected tissues within the eye. These images are then passed, for example, to the computer/comparator for use by the computer/comparator in controlling the laser unit.

Several pertinent structures in the eye are identified and used as reference including the cornea, the sclera, the lens, vitreous body, retina, macula and retinal blood vessels. The vitreous body resides in the vitreous cavity which extends from the retina and macula, posteriorly, to the lens, anteriorly. As such, the vitreous body establishes borders with the lens capsule, retina, macula and retinal blood vessels.

Several situations are of particular interest for the disclosures herein. For one, there is interest in accurately emulsifying target vitreous body tissue at a boundary between the vitreous body and an adjacent anatomical structure. It is to be appreciated that the current discussion is equally applicable to other vitreous body boundaries including boundaries with the lens capsule, retinal blood vessels, the macule, etc.

In some embodiments, use of a computer controlled units with imaging feedback as described herein also allows for more precise targeting. For example, the use of a computer controlled femtosecond laser with imaging feedback as described herein results in a substantial reduction in treatment procedure time or reduction in potential damage.

### Tool Articulation

In some embodiments, the tool tip sits in a robotically controlled articulating region. The articulation region allows movement of the tip of the tool while avoiding motion in the rest of the tool. In some embodiments, the articulation region includes pre-bent tubes, pre-bent tubes recessed within straight or bent tubes, flexures with control wires, flexures fabricated with semiconductor fabrication technologies, and flexures with micro-motors and micro-gears. Use of a robotically controlled articulating tip minimizes the size of the incision in the lens capsule necessary to extract the cataract material. Hence, this is an important technology for capsulorhexis.

An example of an articulating tool is an optical fiber encased in a pre-bent tube, where the pre-bent tube has a rigid, straight exterior tube. In some embodiments, the pre-bent tube is retracted into the straight tube, creating a tool that can change from a bent to a straight configuration. The amount of retraction is controlled robotically, allowing the bend on the tool to be synchronized with the tool pattern and or laser parameters. Use of a pre-bent tube does not limit the articulation means that are used with the tool tip, other means include a flexure with one or more control wires.

In some embodiments, the present invention includes a robot for positioning the tip of the tool in space and optionally providing for angular degrees of freedom for adjusting the direction of the laser tool.

### Matrix to Enhance Lens Stem/Progenitor Cell Growth

In some embodiments, it is desirable to control the porosity of the matrix or biomaterial (e.g., hydrogel) and thus, the ability of nutrients and wastes to diffuse into and out of the matrix. In some embodiments, an appropriate cross-linking agent is added to the aforementioned biomaterial. Several embodiments vary the relative amount of the appropriate cross-linking agent added to the biomaterial resulting in a decrease in average pore size and reduction in diffusion through the hydrogel. Conversely, some embodiments incorporate relatively smaller amounts of cross-linking agent, yielding increased pore size and diffusion through the hydrogel. Several embodiments achieve a balanced degree of structural integrity of the biomaterial and sufficient diffusion of nutrients and wastes.

As used herein, the term “matrix” refers to any substance to which the lens stem cells can adhere and which therefore can substitute the cell attachment function of feeder cells, or supports the adherence thereof such as an attachment factor. Particularly suitable for use with the present invention are extracellular matrix components derived from basement membrane or extracellular matrix components that form part of adhesion molecule receptor-ligand couplings. Non-limiting examples of suitable matrices which can be used by the method of this aspect of the present invention include mammalian amniotic membrane such as human amniotic membrane, collagen (e.g., collagen IV), fibrinogen, perlecan, laminin, fibronectin, proteoglycan, procollagens, hyaluronic acid, entactin, heparan sulfate, tenascin, poly-L-lysine, gelatin, poly-L-ornithin, platelet derived growth factor (PDGF), and the like, or any combinations thereof. Alternatively, the extracellular matrix is commercially provided. Examples of commercially available extracellular matrices are extracellular matrix proteins (Fischer or Life Tech), fibrinogen and thrombin sheet (Reliance Life), and Matrigel™ (BID Biosciences) and their equivalents. In cases where complete animal-free culturing conditions are desired, the matrix is derived from a human source or synthesized using recombinant techniques. Such matrices include, for example, human amniotic membrane, human-derived fibronectin, recombinant fibronectin matrix which can be obtained from Sigma, St. Louis, Mo., USA or can be produced using known recombinant DNA technology (see, for example, U.S. Pat. No. 6,152,142, and Tseng et al., (1997) Am. J. Ophthalmol. 124:765-774, each incorporated herein by reference).

Several embodiments include nutrients, additives and/or growth factors that are added to the biomaterial. Such additives promote cell proliferation, cell differentiation or cell viability. Moreover, in addition to the composition of the biomaterial, additives enhance cell retention. Still other embodiments do not necessitate additive to yield efficacious cell retention. Nutrients, additives and/or growth factors are not limited to those added in an in vitro setting, rather they are released from the cells that are incorporated into the biomaterial or from the local target tissue into/onto which the biomaterial composition is delivered. In addition, in some instances, other nutrients such as glucose, insulin, pyruvate, amino acids, and growth factors are also incorporated into the biomaterial. Still other embodiments include serum supplementation of the biomaterial, with supplementation ranging from about 5-10% serum. In some embodiments, serum supplements the biomaterial at about 7.5%. In some embodiments, serum supplements the biomaterial in a range of about 5-7%, 6-8%, 7-9%, or 8-10%. In some embodiments involving serum supplementation at 7.5%, the biomaterial is hyaluronan. In some embodiments, the biomaterial is supplemented with one or more components associated with the ECM. In some embodiments, the biomaterial is supplemented with collagen. In some embodiments, collagen is added to the biomaterial in a range from about 0.2-0.6% of the final concentration, including 0.3%, 0.4%, and 0.5%. Lower or higher ranges may be used. In some embodiments, about 0.4% collagen is used to supplement hyaluronan to form a cell matrix.

### Computer Systems and Programs

In some embodiments, described herein comprise computer systems or platforms for implementing one or more uses or systems described herein. In some embodiments, also described herein comprise a computer program for controlling a computer system to execute the steps according one or more methods or systems described herein.

In some embodiments, a computer system refers to a system having a computer, where the computer comprises a computer-readable medium embodying software to operate the computer. In some cases, the computer system includes one or more general or special purpose processors and associated memory, including volatile and non-volatile memory devices. In some cases, the computer system memory stores software or computer programs for controlling the operation of the computer system to make a special purpose system according to the invention or to implement a system to perform the methods according to the invention. In some cases, the computer system includes an Intel or AMD x86 based single or multi-core central processing unit (CPU), an ARM processor or similar computer processor for processing the data. In some cases, the CPU or microprocessor is any conventional general purpose single- or multi-chip microprocessor such as an Intel Pentium processor, an Intel 8051 processor, a RISC or MISS processor, a Power PC processor, or an ALPHA processor. In some cases, the microprocessor is any conventional or special purpose microprocessor such as a digital signal processor or a graphics processor. The microprocessor typically has conventional address lines, conventional data lines, and one or more conventional control lines. As described below, the software according to the invention is executed on dedicated system or on a general purpose computer having a DOS, CPM, Windows, Unix, Linix or other operating system. In some instances, the system includes non-volatile memory, such as disk memory and solid state memory for storing computer programs, software and data and volatile memory, such as high speed ram for executing programs and software.

In some embodiments, a computer-readable medium refers to any storage device used for storing data accessible by a computer, as well as any other means for providing access to data by a computer. Examples of a storage device-type computer-readable medium include: a magnetic hard disk; a floppy disk; an optical disk, such as a CD-ROM and a DVD; a magnetic tape; a memory chip. Computer-readable physical storage media useful in various embodiments of the invention can include any physical computer-readable storage medium, e.g., solid state memory (such as flash memory), magnetic and optical computer-readable storage media and devices, and memory that uses other persistent storage technologies. In some embodiments, a computer readable media is any tangible media that allows computer programs and data to be accessed by a computer. Computer readable media can include volatile and nonvolatile, removable and non-removable tangible media implemented in any method or technology capable of storing information such as computer readable instructions, program modules, programs, data, data structures, and database information. In some embodiments of the invention, computer readable media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and nonvolatile memory, and any other tangible medium which can be used to store information and which can read by a computer including and any suitable combination of the foregoing.

In some instances, one or more methods described herein are implemented on a stand-alone computer or as part of a networked computer system or computing platform. In a stand-alone computer, all the software and data can reside on local memory devices, for example an optical disk or flash memory device can be used to store the computer software for implementing the invention as well as the data. In alternative embodiments, the software or the data or both can be accessed through a network connection to remote devices.

In some instances, computer instructions are implemented in software, firmware or hardware and include any type of programmed step undertaken by modules of the information processing system. In some cases, the computer system is connected to a local area network (LAN) or a wide area network (WAN). One example of the local area network can be a corporate computing network, including access to the Internet, to which computers and computing devices comprising the data processing system are connected. In one embodiment, the LAN uses the industry standard Transmission Control Protocol/Internet Protocol (TCP/IP) network protocols for communication. Transmission Control Protocol Transmission Control Protocol (TCP) can be used as a transport layer protocol to provide a reliable, connection-oriented, transport layer link among computer systems. The network layer provides services to the transport layer. Using a two-way handshaking scheme, TCP provides the mechanism for establishing, maintaining, and terminating logical connections among computer systems. TCP transport layer uses IP as its network layer protocol. Additionally, TCP provides protocol ports to distinguish multiple programs executing on a single device by including the destination and source port number with each message. TCP performs functions such as transmission of byte streams, data flow definitions, data acknowledgments, lost or corrupt data retransmissions, and multiplexing multiple connections through a single network connection. Finally, TCP is responsible for encapsulating information into a datagram structure. In alternative embodiments, the LAN can conform to other network standards, including, but not limited to, the International Standards Organization's Open Systems Interconnection, IBM's SNA, Novell's Netware, and Banyan VINES.

### Server

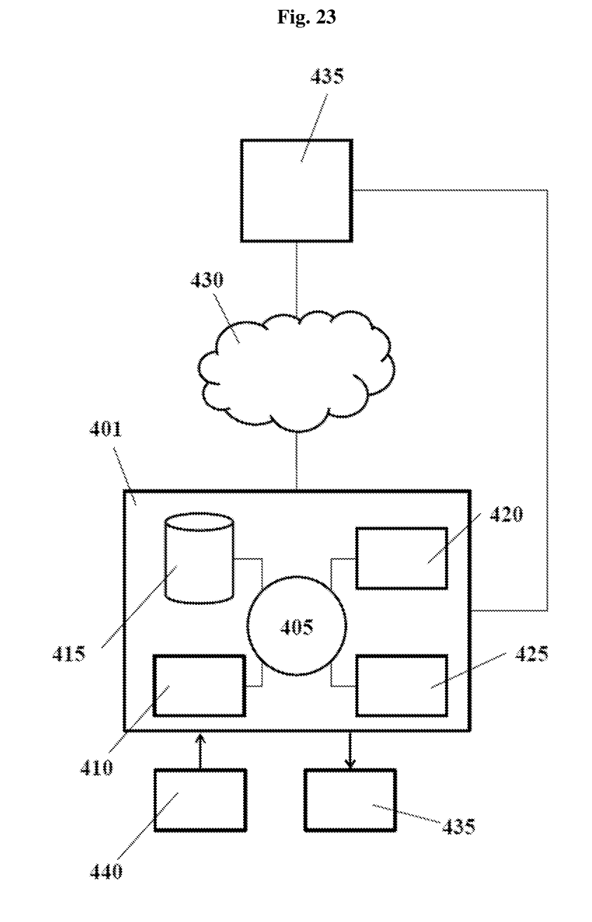

In some embodiments, the methods and systems provided herein are processed on a server or a computer server (FIG. 23). In some embodiments, the server 401 includes a central processing unit (CPU, also “processor”) 405 which is a single core processor, a multi core processor, or plurality of processors for parallel processing. In some embodiments, a processor used as part of a control assembly is a microprocessor. In some embodiments, the server 401 also includes memory 410 (e.g. random access memory, read-only memory, flash memory); electronic storage unit 415 (e.g. hard disk); communications interface 420 (e.g. network adaptor) for communicating with one or more other systems; and peripheral devices 425 which includes cache, other memory, data storage, and/or electronic display adaptors. The memory 410, storage unit 415, interface 420, and peripheral devices 425 are in communication with the processor 405 through a communications bus (solid lines), such as a motherboard. In some embodiments, the storage unit 415 is a data storage unit for storing data. The server 401 is operatively coupled to a computer network (“network”) 430 with the aid of the communications interface 420. In some embodiments, a processor with the aid of additional hardware is also operatively coupled to a network. In some embodiments, the network 430 is the Internet, an intranet and/or an extranet, an intranet and/or extranet that is in communication with the Internet, a telecommunication or data network. In some embodiments, the network 430 with the aid of the server 401, implements a peer-to-peer network, which enables devices coupled to the server 401 to behave as a client or a server. In some embodiments, the server is capable of transmitting and receiving computer-readable instructions (e.g., device/system operation protocols or parameters) or data (e.g., sensor measurements, raw data obtained from detecting metabolites, analysis of raw data obtained from detecting metabolites, interpretation of raw data obtained from detecting metabolites, etc.) via electronic signals transported through the network 430. Moreover, in some embodiments, a network is used, for example, to transmit or receive data across an international border.

In some embodiments, the server 401 is in communication with one or more output devices 435 such as a display or printer, and/or with one or more input devices 440 such as, for example, a keyboard, mouse, or joystick. In some embodiments, the display is a touch screen display, in which case it functions as both a display device and an input device. In some embodiments, different and/or additional input devices are present such an enunciator, a speaker, or a microphone. In some embodiments, the server uses any one of a variety of operating systems, such as for example, any one of several versions of Windows®, or of MacOS®, or of Unix®, or of Linux®.

In some embodiments, the storage unit 415 stores files or data associated with the operation of a device, systems or methods described herein.

In some embodiments, the server communicates with one or more remote computer systems through the network 430. In some embodiments, the one or more remote computer systems include, for example, personal computers, laptops, tablets, telephones, Smart phones, or personal digital assistants.

In some embodiments, a control assembly includes a single server 401. In other situations, the system includes multiple servers in communication with one another through an intranet, extranet and/or the Internet.

In some embodiments, the server 401 is adapted to store device operation parameters, protocols, methods described herein, and other information of potential relevance. In some embodiments, such information is stored on the storage unit 415 or the server 401 and such data is transmitted through a network.

### Certain Terminology

While preferred embodiments of the present disclosure have been shown and described herein, it will be obvious to those skilled in the art that such embodiments are provided by way of example only. Numerous variations, changes, and substitutions will occur to those skilled in the art without departing from the present disclosure. It should be understood that various alternatives to the embodiments of the present disclosure described herein may be employed. It is intended that the following claims define the scope of the present disclosure and that methods and structures within the scope of these claims and their equivalents be covered thereby.

Unless defined otherwise, all technical and scientific terms used herein have the same meaning as commonly understood by one of skill in the art to which the present disclosure described herein belong. All publications, patents, and patent applications mentioned in this specification are hereby incorporated by reference to the same extent as if each individual publication, patent, or patent application was specifically and individually indicated to be incorporated by reference.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose. Additionally, the structures, systems, and/or devices described herein may be embodied as integrated components or as separate components. Accordingly, the methods and systems of the present invention are not limited to cataract surgery and other ophthalmologic applications.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, e.g., ±5%, ±10% or ±15%.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

Embodiment one refers to a method of expanding lens epithelial stem and progenitor cells in situ, comprising: (i) making a capsulorhexis opening in a peripheral area of lens anterior capsule of an eye of a subject; (ii) removing contents of the lens; and (iii) administering into the anterior capsule through the capsulorhexis opening a biomaterial composition to maintain the structural integrity of the anterior capsule and to induce expansion of lens epithelial stem and progenitor cells in situ.

Embodiment two refers to embodiment one, wherein the biomaterial composition comprises human serum and a fibroblast growth factor (FGF).

Embodiment three refers to embodiments one or two, wherein the biomaterial composition further comprises a nutrient, an additive, or a combination thereof, wherein the nutrient comprises a composition of amino acids and optionally one or more nutrients, and wherein the additive comprises calcium chloride, potassium chloride, magnesium sulfate, sodium chloride, monosodium phosphate, potassium phosphate, sodium bicarbonate, sodium phosphate, or a combination thereof.

Embodiment four refers to embodiment one, wherein the capsulorhexis opening is about 1.0 to 2.0 mm in diameter.

Embodiment five refers to embodiment one, wherein the capsulorhexis opening is about 1.0 to 1.5 mm in diameter.

Embodiment six refers to embodiment one, wherein the capsulorhexis opening is located away from the central visual axis of the eye.

Embodiment seven refers to embodiment one, wherein the subject has cataract.

Embodiment eight refers to embodiment one, wherein the subject is an animal or human.

Embodiment nine refers to embodiment eight, wherein the human is aged 18 or older.

Embodiment ten refers to embodiment eight, wherein the human is aged 17 or younger.

Embodiment eleven refers to embodiment ten, wherein the human has a pediatric cataract.

Embodiment twelve refers to embodiment eight, wherein the human is an adult or an infant.

Embodiment thirteen refers to embodiment twelve, wherein the human infant has congenital cataract.

Embodiment fourteen refers to embodiment seven, wherein cataract is removed.

Embodiment fifteen refers to embodiment one, wherein the lens epithelial stem and progenitor cells express Pax6 and/or Bmi-1.

Embodiment sixteen refers to embodiment one, wherein the method does not involve an implantation of an artificial intraocular lens (IOL).

Embodiment seventeen refers to embodiment one, wherein the method results in reduced visual axis opacification (VAO) relative to a method comprising a capsulorhexis procedure comprising central capsulorhexis opening and implantation of an artificial intraocular lens.

Embodiment eighteen refers to embodiment one, wherein the method results in lowered incidents of complications selected from the group consisting of corneal edema, anterior chamber inflammation, and visual axis opacification.

Embodiment nineteen refers to a system for performing a minimally invasive method of cataract removal, comprising an imaging unit, a phacoemulsification unit for emulsifying cataract material, an aspiration unit for removing cataract material, and a biomaterial delivery unit for delivering a biomaterial composition into a capsular bag via a lens capsule opening, wherein all of the units are operationally connected to a computer.

Embodiment twenty refers to embodiment nineteen, wherein the phacoemulsification unit comprises an ultrasound or laser probe, said probe is equipped with a tip designed to be inserted into a peripheral area of lens anterior capsule of an eye.

Embodiment twenty-one refers to embodiment twenty, wherein the tip is configured to perform one or both of making an opening of about 1.0 to 2.0 mm in diameter and removing cataract from the eye.

Embodiment twenty-two refers to embodiment twenty, wherein the tip is configured to perform one or both of making an opening of about 1.0 to 1.5 mm in diameter and removing cataract from the eye.

Embodiment twenty-three refers to embodiment twenty, wherein the tip is configured to prevent damage to endogenous lens epithelial stem and progenitor cells.

Embodiment twenty-four refers to embodiment nineteen, wherein the imaging unit employs imaging technique selected from the group consisting of 3D imaging, optical coherence tomography, MRI, CT, and ultrasound.

Embodiment twenty-five refers to embodiment nineteen, wherein the biomaterial composition comprises human serum and a fibroblast growth factor (FGF).

Embodiment twenty-six refers to embodiment nineteen, wherein the biomaterial composition further comprises a nutrient, an additive, or a combination thereof, wherein the nutrient comprises a composition of amino acids and optionally one or more nutrients, and wherein the additive comprises calcium chloride, potassium chloride, magnesium sulfate, sodium chloride, monosodium phosphate, potassium phosphate, sodium bicarbonate, sodium phosphate, or a combination thereof.

Embodiment twenty-seven refers to a use of a system of embodiments nineteen to twenty-six for removing cataract in a subject in need thereof.

Embodiment twenty-eight refers to embodiment twenty-seven, wherein the subject is an animal or human.

Embodiment twenty-nine refers to embodiment twenty-eight, wherein the human is aged 18 or older.

Embodiment thirty refers to embodiment twenty-eight, wherein the human is aged 18 or younger.

Embodiment thirty-one refers to embodiment thirty, wherein the human has a pediatric cataract.

Embodiment thirty-two refers to embodiment twenty-eight, wherein the human is an adult or an infant.

Embodiment thirty-three refers to embodiment thirty-two, wherein the human infant has congenital cataract.

Embodiment thirty-four refers to a method of lens regeneration using endogenous lens epithelial stem and progenitor cells, comprising the steps of: (i) isolating lens epithelial stem and progenitor cells in the anterior capsule of an eye of a subject; and (ii) contacting the lens epithelial stem and progenitor cells in the anterior capsule with a biomaterial composition, wherein the stem and progenitor cells proliferate and differentiate into lens fiber cells to form a lens.

Embodiment thirty-five refers to embodiment thirty-four, wherein the biomaterial composition comprises human serum and a fibroblast growth factor (FGF).

Embodiment thirty-six refers to embodiments thirty-four or thirty-five, wherein the biomaterial composition further comprises a nutrient, an additive, or a combination thereof, wherein the nutrient comprises a composition of amino acids and optionally one or more nutrients, and wherein the additive comprises calcium chloride, potassium chloride, magnesium sulfate, sodium chloride, monosodium phosphate, potassium phosphate, sodium bicarbonate, sodium phosphate, or a combination thereof.

Embodiment thirty-seven refers to embodiment thirty-four, further comprising making a capsulorhexis opening in a peripheral area of the lens anterior capsule.

Embodiment thirty-eight refers to embodiment thirty-seven, wherein the capsulorhexis opening is about 1.0 to 2.0 mm in diameter.

Embodiment thirty-nine refers to embodiment thirty-seven, wherein the capsulorhexis opening is about 1.0 to 1.5 mm in diameter.

Embodiment forty refers to embodiment thirty-seven, wherein the capsulorhexis opening is located away from the central visual axis of the eye.

Embodiment forty-one refers to embodiment thirty-four, wherein the subject has cataract.

Embodiment forty-two refers to embodiment thirty-four, wherein the subject is an animal or human.

Embodiment forty-three refers to embodiment forty-two, wherein the human is aged 18 or older.

Embodiment forty-four refers to embodiment forty-two, wherein the human is aged 17 or younger.

Embodiment forty-five refers to embodiment forty-four, wherein the human has a pediatric cataract.

Embodiment forty-six refers to embodiment forty-two, wherein the human is an adult or an infant.

Embodiment forty-seven refers to embodiment forty-six, wherein the human infant has congenital cataract.

Embodiment forty-eight refers to embodiment thirty-four, wherein the isolating of lens epithelial stem and progenitor cells in step (i) comprises selecting or enriching stem and progenitor cells that express Pax6 and Bmi-1.

Embodiment forty-nine refers to embodiment one, wherein the biomaterial composition is administered in a volume sufficient to replace the volume lost due to the removal of the contents of the lens from the lens anterior capsule.

Embodiment fifty refers to embodiment nineteen, wherein the biomaterial composition is administered in a volume sufficient to replace the volume lost due to the removal of the cataract material from the capsular bag.

### EXAMPLES

These examples are provided for illustrative purposes only and not to limit the scope of the claims provided herein.

### Example 1—Lens Regeneration Using Endogenous Progenitor Cells with Gain of Visual Function

Lens epithelial progenitor cells (LECs) in mammals were identified and isolated. It was shown that Pax6 and Bmi-1 are required for LEC renewal and proliferation. This example also describes a surgical method for cataract removal that preserves the integrity of the lens capsule and its associated endogenous LECs. Using this method, functional lens regeneration was achieved in rabbits and macaques, as well as in human infants with cataract. The surgical method described herein conceptually differs from current practice, as it maximally preserves endogenous LECs and their natural environment, and regenerates lenses with visual function. These findings exemplify a novel treatment strategy for cataract and provide a new paradigm for tissue regeneration using endogenous progenitor cells.

Isolation And Culture of LECs

All animal studies were performed with the approval of the Institutional Animal Care Committees of Sun Yat-sen University, the University of California San Diego, West China Hospital, and the University of Texas Southwestern Medical Center.

The eyeball was enucleated from a one-month-old New Zealand white rabbit and washed with PBS (containing antibiotics) three times. After the cornea and iris were removed, a small cut was made in the posterior capsule of the lens; the capsule with attached epithelium was removed and cut into 1×1 mm² pieces. The pieces of epithelium were cultured in minimum essential media supplemented with 20% FBS, NEAA, and 50 μg/ml gentamicin.

A 17-week-old human fetal eyeball was purchased from Advanced Bioscience Resources, Inc. (San Francisco, Calif.). The LECs were cultured according to the same methods as above.

For in vitro differentiation, LECs were cultured on Matrigel-coated 6-well plates or 8-well chambers. Lentoid body was formed after 21 days in minimum essential media supplemented with NEAA, 1% FBS, 100 ng/mL FGF2, and 5 μg/mL insulin. Images of lentoid tissue were obtained using a Leica M205FA stereo microscope.

### Transgenic Mouse Study

Membrane-tomato/membrane-green (mTmG)-targeted ROSA^{mTmG} mice were purchased from the Jackson Laboratory (Bar Harbor, Me.; stock no. 7576) and maintained as homozygotes. P0-3.9-GFPCre mice expressing an EGFP-Cre recombinase fusion protein under the control of the Pax6 lens ectoderm enhancer and the Pax6 P0 promoter²⁶ were maintained in a FVB/N background. Lineage-tracing experiments were performed by crossing the homozygous ROSA^{mTmG} reporter mouse strain with the P0-3.9-GFPCre deleted strain. Eyes were dissected at P1, P14, and P30 and fixed overnight in 4% formaldehyde. Tissues were then incubated in 10% sucrose and embedded in OCT for cryosectioning. Frozen sections were washed in PBS and imaged on a Zeiss Axio Imager fluorescence microscope. Bmi-1^{fl/fl} mice were generated as previously described²⁷. Nestin-Cre mice²⁸ were obtained from the Jackson Laboratory. For BrdU pulses, mice were injected with 100 mg/kg BrdU (Sigma) dissolved in PBS, then maintained on drinking water that contained 1 mg/ml BrdU until sacrifice.

For gene expression study, lenses of Pax6P0-3.9-GFPCre mice were dissected under a dissecting microscope. Lens capsular bag was opened from the posterior surface by making three crisscross incisions. The capsular bag was opens and lens material extruded. GFP-positive LECs in the mid-anterior capsular area were separated mechanically from GFP-negative LECs in the remaining capsular areas under a fluorescence microscope. RNA was isolated using RNeasy Mini Kit (Qiagen).

To image cataracts, mice were anesthetized with Avertin, and one drop of 1% Mydriacyl (Alcon) was administered per eye. Eyes were immediately visualized in vivo using a light microscope. For histology, mice were perfused with heparinized saline followed by 4% paraformaldehyde (PFA) in PBS. Dissected eyes were fixed in 4% PFA overnight, embedded in paraffin, and sectioned by the UT Southwestern Molecular Pathology core facility. For BrdU staining, slides were deparaffinized, and subjected to heat-mediated antigen retrieval (in 10 mM sodium citrate, pH 6.0). Slides were stained with primary mouse anti-BrdU (Caltag, MD5000, 1:200) overnight at 4° C. Slides were subsequently stained with Alexa Fluor 555-conjugated goat anti-mouse IgG1 secondary antibody (Life Technologies, 1:500) and 1 mg/ml DAPI (1:500) for 1 h at room temperature. The number of BrdU-labeled cells was divided by the total number of DAPI+ cells in a single layer of LECs.

### Lentiviral RNAi

Lentiviral shRNA targeting the human BMI-1 gene (NCBI Reference Sequence: NM_005180.8) was purchased from Origene (TL314462), ShRNA targeting sequences were as follow: 5′-AATGCCATATTGGTATATGAC-ATAACAGG-3′ (SEQ ID NO: 31) and 5′-GTAAGAATCAG ATGGCATTATGCTTGTTG-3′ (SEQ ID NO: 32). Two shRNAs were used separately, and a non-effective 29-mer scrambled shRNA was used as a control. Lentiviral shRNA particles were prepared using shRNA lentiviral packaging kit (Origene, TR30022). Viruses were harvested at 48 h and 72 h post-transfection.

### Western Blot Analysis

LECs were cultured on Matrigel-coated 3.5 mm dishes with lentoid formation medium for 30 days. Cells were washed twice with ice-cold PBS, and lysed in RIPA lysis buffer with PMSF. Protein concentration was determined by BCA protein assay kit. 30 μg of total protein lysate was loaded onto 10% SDS-PAGE gel and then transferred to a PVDF membrane (Millipore) at 70V for 2 h. The membrane was probed with the following primary antibody at 4° C. overnight: anti-αA-crystallin (sc-22389, Santa Cruz), anti-β-crystallin (sc-48335, Santa Cruz), anti-γ-crystallin (sc-22415, Santa Cruz) and anti-β-actin (sc-47778, Santa Cruz), and then incubated with HRP-conjugated anti-rabbit, anti-mouse, or anti-goat secondary antibody for 1 h at room temperature. The immunodetection was visualized using a blot imaging system (Fluor Chem Q, Protein Simple) with ECL buffer (Millipore).

### Lens Regeneration in Rabbit and Macaque Models

New Zealand white rabbits (n=29, 4 rabbits died from systemic infections unrelated to surgery. The remaining 25 rabbits were used to assess regeneration), and long-tailed macaques (M. fascicularis) monkeys (n=6) underwent minimally invasive capsulorhexis surgery. Only the left eye of each animal was used for experiments. Slit-lamp biomicroscopy and photography were performed at different time points to monitor lens regeneration. Rabbits were sacrificed at day 1, day 7, and one month after surgery, and the treated eyes were enucleated. The lenses were harvested for histologic analysis using H&E staining. For the macaques, enucleation of the treated eye was performed 4 months post-surgery and the lenses were harvested for the same histologic examinations. The eyes were fixed, paraffin-embedded, and sectioned at 5 μm through the cornea, pupil, and optic nerve with the lens in situ.

### Real-Time PCR

RNA was isolated from rabbit LECs, mature lens fiber cells and LECs in P0-3.9-GFPCre mice using an RNeasy Mini Kit (Qiagen) and subjected to on-column DNase digestion. cDNA was synthesized using a Superscript III reverse transcriptase kit according to the manufacturer's instructions (Invitrogen). Quantitative PCR was performed via 40 cycle amplification using gene-specific primers (Table 2) and Power SYBR Green PCR Master Mix on a 7500 Real-Time PCR System (Applied Biosystems). Measurements were performed in triplicate and normalized to endogenous GAPDH levels. The relative fold change in expression was calculated using the ΔΔCT method (CT values<30).

### Immunofluorescence and Laser Confocal Microscopy

Rabbit LECs were fixed in 4% PFA for 20 min, then permeabilized with 0.3% Triton X-100-PBS for 10 min and blocked in PBS solution containing 5% BSA, followed by an overnight incubation in primary antibodies at 4° C. After 3 washes in PBS, cells were incubated with secondary antibody for 1 h in room temperature. Cell nuclei were counterstained with DAPI.

The following antibodies were used: goat anti-Sox2 polyclonal antibody (Santa Cruz), rabbit anti-PAX6 polyclonal antibody (PRB-278P, Covance), mouse anti-Bmi1 antibody (ab14389, Abcam), and mouse anti-Ki67 monoclonal antibody (550609, BD Sciences). The secondary antibodies, Alexa Fluor 488- or 568-conjugated anti-mouse or anti-rabbit IgG (Invitrogen), were used at a dilution of 1:500. Images were obtained using an Olympus FV1000 confocal microscope.

### BrdU Labeling of LECs in Humans

BrdU labeling was used to identify and quantify proliferating LECs from human cadaver eyes. Whole-mount human lens capsules were pulsed with BrdU and then stained with an antibody against BrdU to determine the distribution and density of proliferating LECs. In brief, within 12-24 hours after death, lenses from postmortem donor eyes were obtained from the Eye Bank of Zhongshan Ophthalmic Center in Guangzhou, China. Twelve lenses in total from six donors were used for the experiment. A small puncture injury was made on the anterior surface of a postmortem human lens using a 30-gauge needle. The lenses were cultured at 37° C. in Dulbecco modified Eagle medium (DMEM) supplemented with 10% FBS in a humidified incubator with 5% CO₂. The contralateral lens from the same donor was treated under the same conditions but did not receive a puncture injury and was used as a control. To label the proliferating LECs, both groups of lenses were incubated in 100 μg/ml BrdU (Sigma-Aldrich) 24 hours after the puncture injury. The lens was then removed from the capsular bag, and the lens capsules were fixed in 4% formaldehyde and subjected to BrdU staining using a standard immunohistochemistry protocol according to the manufacturer's instructions (CST, Boston, Mass.). Images were taken using a Carl Zeiss microscope (Jena, Germany).

### Study Design, Execution, and Oversight of Clinical Trial in Humans

This study was approved by the institutional review board of the Zhongshan Ophthalmic Center (ZOC). Informed written consent was obtained from the parents or guardians of the infants before enrollment, and the tenets of the Declaration of Helsinki were followed throughout the study. The study was conducted in accordance with an international guideline and protocol for visual function measurements in pediatric cataract surgery and a protocol of the Childhood Cataract Program of the Chinese Ministry of Health (CCPMOH) and had an independent data and safety monitoring board of ZOC-CCPMOH.

### Description of Current Surgical Method for Cataract Extraction

The current standard-of-care treatment for pediatric cataract involves removal of the cataractous lens through a relatively large opening using anterior continuous curvilinear capsulorhexis (ACCC, about 6 mm in diameter; FIG. 1), followed by cataract extraction and artificial lens implantation or placement of postoperative aphakic eyeglasses/contact lens in pediatric cataract patients younger than two years. Some patients underwent additional posterior continuous curvilinear capsulorhexis (PCCC) and anterior vitrectomy.

### Establishment of a Minimally Invasive Capsulorhexis Surgery Method to Preserve LECs

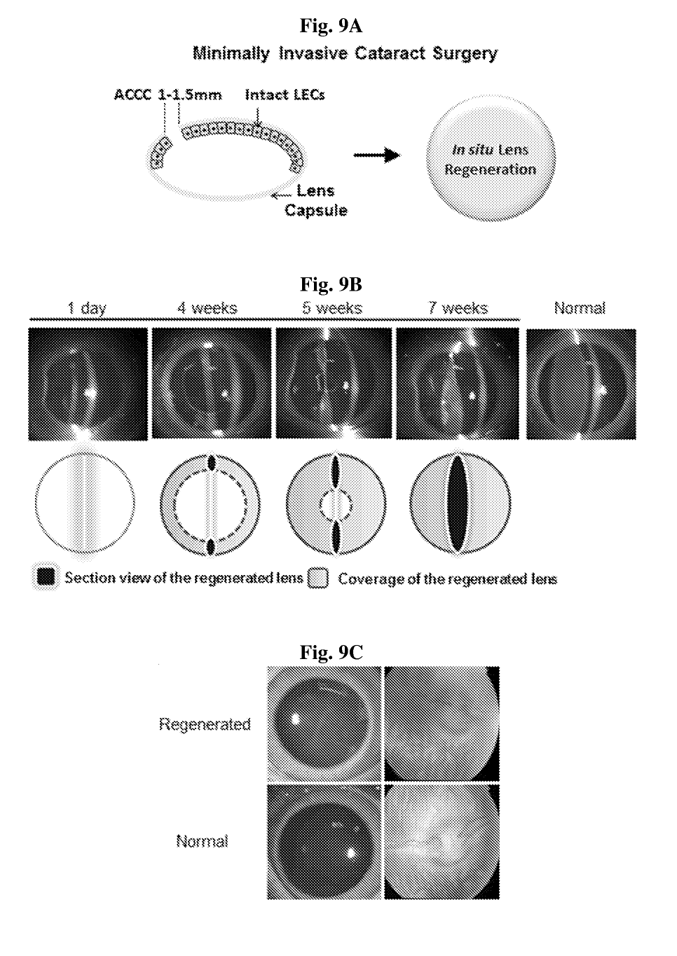

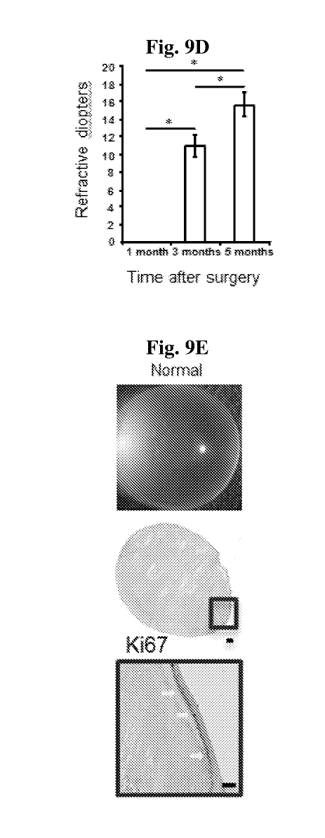

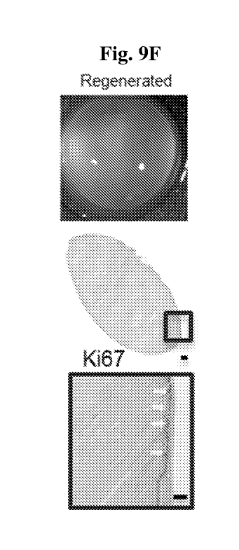

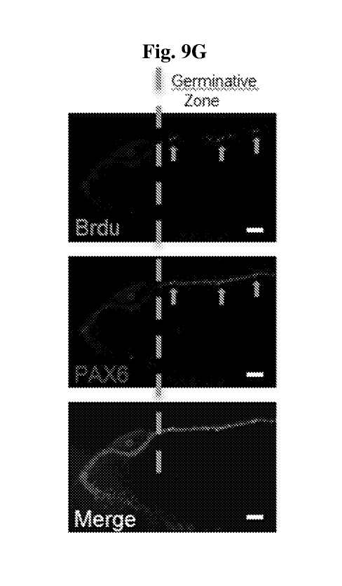

A new capsulorhexis surgery method was established to facilitate lens regeneration (FIG. 9A). First, we decreased the size of the cap sulorhexis opening to 1.0-1.5 mm in diameter. This results in a minimal wound of about 1.2 mm² in area, which is only about 4.3% the size of the wound created by the current method. Second, we moved the location of the capsulorhexis to the peripheral area of the lens instead of the central area. A 0.9 mm phacoemulsification probe was used to remove the lens contents and/or cortical opacities. These changes provide significant advantages. First, it considerably reduces the size of the injury, which resulted in a lower incidence of inflammation and much faster healing. Second, it moves the wound scar away from the central visual axis to the periphery, leading to improved visual axis transparency. Third, it preserves a nearly intact transparent lens capsule and layer of LECs, which have regenerative potential and are critically required for the regeneration of a natural lens.

Clinical Trial of Minimally Invasive Lens Surgery in Human Infants with Congenital Cataract

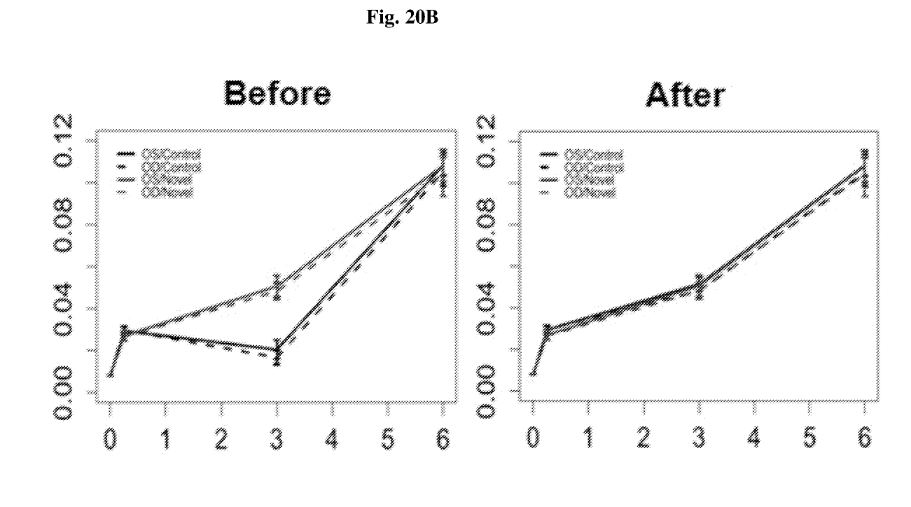

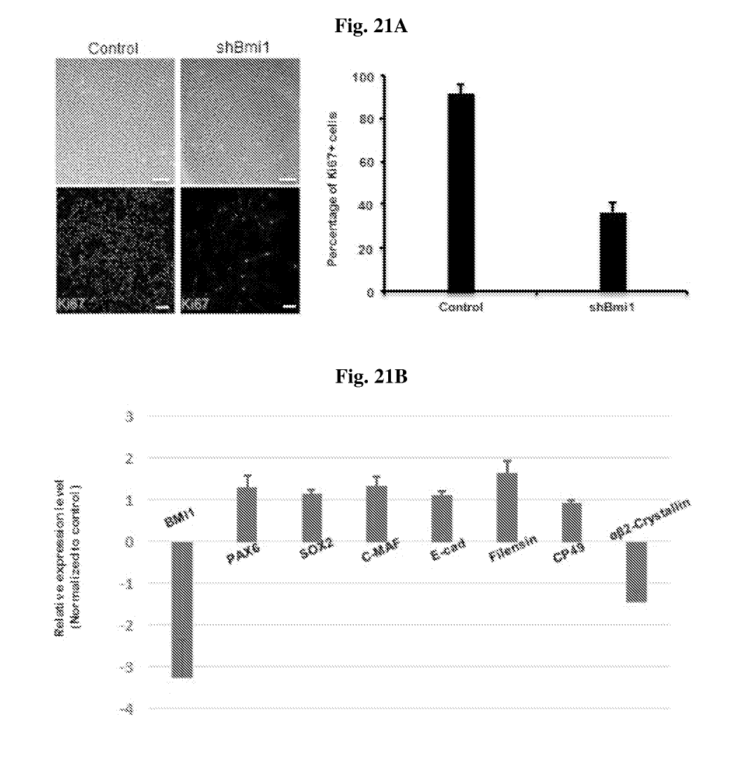

Pediatric patients were selected from the Childhood Cataract Program of the Chinese Ministry of Health (CCPMOH), which includes a series of studies on the influence of early interventions on the long-term outcomes of pediatric cataract treatment (ClinicalTrials.gov Identifier: NCT01844258). Inclusion criteria were the following: Infants were ≤24 months old, and diagnosed with bilateral or unilateral uncomplicated congenital cataract with an intact non-fibrotic capsular bag. Exclusion criteria included preoperative intraocular pressure (IOP)>21 mmHg, premature birth, family history of ocular disease, ocular trauma, or other abnormalities, such as microcornea, persistent hyperplastic primary vitreous, rubella, or Lowe syndrome. Twelve pediatric cataract patients (24 eyes) received the new minimally invasive lens surgery (Table 1 and Table 3). Twenty-five pediatric cataract patients (50 eyes in total) were enrolled as the control group to receive the current standard surgical treatment (FIG. 20A). A clinical trial consort flowchart is listed in FIG. 20A.

The incidence of corneal edema was defined as >5% increase in central corneal thickness one week post-surgery, and the incidence of severe anterior chamber inflammation as Flare value>10 evaluated by Pentacam system (OCULUS, Germany) and Laser flare meter (KOWA FM-600, Japan). Early-onset ocular hypertension was identified as IOP>21 mmHg by Tonopen (Reichert, Seefeld, Germany) within 1 month after surgery. Macular edema was identified by fundus OCT (iVue, Optovue, Germany) as an increase in central macular thickness>10% one week post-surgery. When indicated, VAO, defined by visual decline and the degree to which the fundus was obscured, was treated with YAG laser capsulotomy at follow-up.

Compared to infants operated on using the new surgical technique described herein, infants who received the traditional technique had a higher incidence of anterior chamber inflammation one week after surgery, early-onset ocular hypertension, and increased VAO (Table 1 and Table 3). However, in the group treated with the present new method, a transparent regenerated biconvex lens was found in 100% of eyes 3 months after surgery, while no regenerated biconvex lenses formed in the group treated with the standard technique. In addition, 100% of the capsular openings healed within 1 month after surgery in the experimental group, but no capsular openings healed in the control group.

### Evaluation of Pediatric Visual Acuity

Testing equipment included a set of Teller Acuity Cards (Vistech Consultants, Dayton, Ohio). The set of cards consists of 15 cards with gratings ranging in spatial frequency from 0.32 to 38 cycles/cm, in half-octave steps, and one blank gray card. A 4-mm peephole in each card allows the tester to view the child's face through the card during testing. Test distance was kept constant by use of an aid to measure the distance from the child's eyes to the card throughout testing. For 38 cm, the aid was the distance measured from the tester's elbow to a specific knuckle on the tester's hand, and for 55 cm, the aid was the length (55 cm) of the Teller Acuity Card. Testers were instructed to hold the cards without wrapping their fingers around the front side of the card, as this may attract the child's attention. Testers presented the cards directly in front of the child and observed the child either over the top of the card or through the peephole in the card.

During each acuity test, testers were aware that the gratings were arranged in order from lower to higher spatial frequencies in half-octave steps, but were masked to the absolute spatial frequency of the grating on each card. The subset of spatial frequencies used for each test was selected according to a pseudorandom order from among three possible subsets of spatial frequencies for the subject's age group. All three subsets for each age group included spatial frequencies known to be well above threshold for that age group. To keep the tester masked to the absolute spatial frequency, the tester was not permitted to look at the front of the card to confirm the location of the grating. Instead, the tester asked an assistant to confirm the location of the grating on the card, after the tester had shown a card to the subject enough times to assess whether or not the subject could detect the grating. Testers were masked to the acuity results until each subject had completed testing. Acuity was scored as the spatial frequency of the finest grating that the tester judged the child could see, based on his/her eye and head movement responses to each card presented. Acuity scores were converted to log values prior to data analysis.

### Measurement of Lens Refractive Power

A handheld auto-refractometer (PlusoptiX A09, OptiMed, Sydney, Australia) was used to evaluate the function of the regenerated lenses according to the manufacturer's methods.

### Statistical Analysis

To determine if visual acuity improved in eyes treated with the new minimally invasive surgery, ANOVA was performed to compare visual acuity preoperatively and at multiple time points postoperatively. If Levene's test failed to demonstrate homogeneity of variances, then Kruskal-Wallis tests were used instead. Pairwise comparisons were performed to evaluate for significant improvement in visual acuity compared to preoperative baseline. In addition, for each time point before and after surgery, t-tests were used to compare the visual acuity of the group receiving traditional surgery to that of the group receiving the new minimally invasive surgery.

Descriptive statistics was provided for the primary and secondary endpoints measured by intervention groups at each time point. Mean and standard deviation was reported for continuous variable and count and percentage is reported for categorical variable. To assess whether the primary outcome, decimal acuity, was significantly improved within each group, pre-post comparison was performed between decimal acuity measured at baseline and study endpoint using paired t-test. Normality of the data was checked and nonparametric alternatives, Wilcoxon signed-rank test is considered if the assumption was severely violated. To evaluate whether the mean response profiles in two groups were similar, linear mixed-effect model was used with account for the within-subject correlation. The baseline decimal acuity was not adjusted by the model due to the homogeneity of this measurement. As the standard-of-care approach requires a laser surgery at 3 month while the novel treatment does not, two models were fit using before- and after-laser surgery data, separately, to demonstrate the superiority of the novel approach. In each model, the outcome is the decimal acuity measured at four time-points: baseline, 1 week, 3 months (before- or after-laser surgery) and 6 months; time (baseline as the reference level), treatment assignment and their interaction are the fixed effects; and patient is the random effect. Significant associations are identified using likelihood ratio test (LRT) by comparing models with and without a fixed effect. A linear mixed-effect model is fit again by dropping out the insignificant fixed effect until the final model is selected. Contrasts test is performed when necessary.

For the secondary aim, the proportions of each condition of complications were compared between two groups. The occurrence of complications for eyes from the same patient was assumed to be independent. The mean difference and its 95% confidence interval was reported. A two-proportion z-test was used with the nonparametric Chi-squared test as alternative if the normality assumption was violated. All tests were two-sided and a p-value less than 0.05 is considered to be statistically significant.

### Evaluation of Accommodative Response

Accommodative response was measured by an open-field autorefractor (SRW-5001K; Shin-Nippon, Tokyo, Japan), which allows targets to be viewed at any distance. The pediatric patients were positioned for autorefractor measurement with assistance from their parents. The patients were guided to fixate binocularly at a near target (33 cm, 5×5 array of smiley faces of N10 size) and a far target (3 m, 5×5 array of smiley faces of N10 size) by a trained and certified investigator or study coordinator. The measurements from non-cycloplegic autorefraction were performed three times at each target distance by the same trained and certified investigator throughout the study, in order to maintain accuracy and consistency throughout the trial. Measurements were taken in the same quiet environment with consistent room illumination to diminish influence of distracting factors and to maintain subjects' concentration. The spherical equivalent refractive value (SER) was recorded for each measurement and the mean value was calculated for evaluation of an accommodative response. The value of accommodative response was the difference between SER values for the near and the far target. We also used dynamic retinoscopy to measure the infants' accommodation. Briefly, we recorded a lens diopter value using retinoscopy when a patient was guided to fixate on a target 3 m away. Then another lens diopter value was recorded when the target was moved closer, at a distance of 33 cm from the eyes. The difference between these two measurements was used to evaluate lens accommodative power.

### Role of LECs in Lens Regeneration

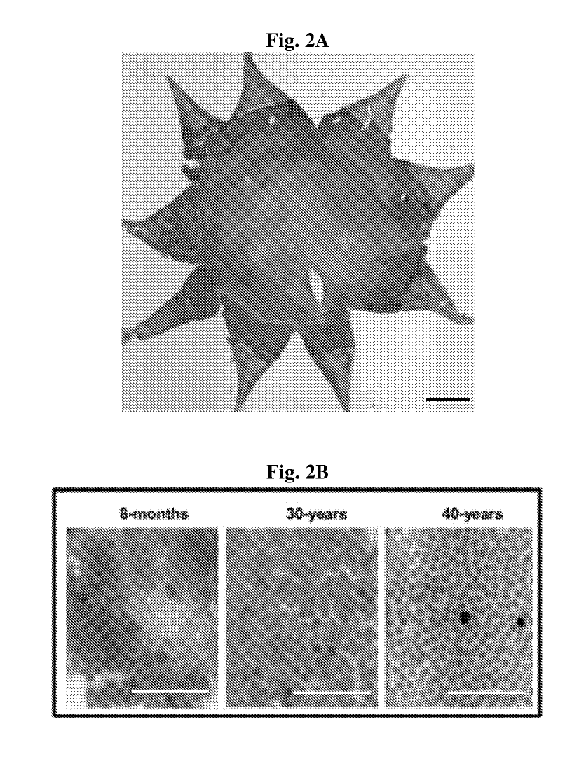

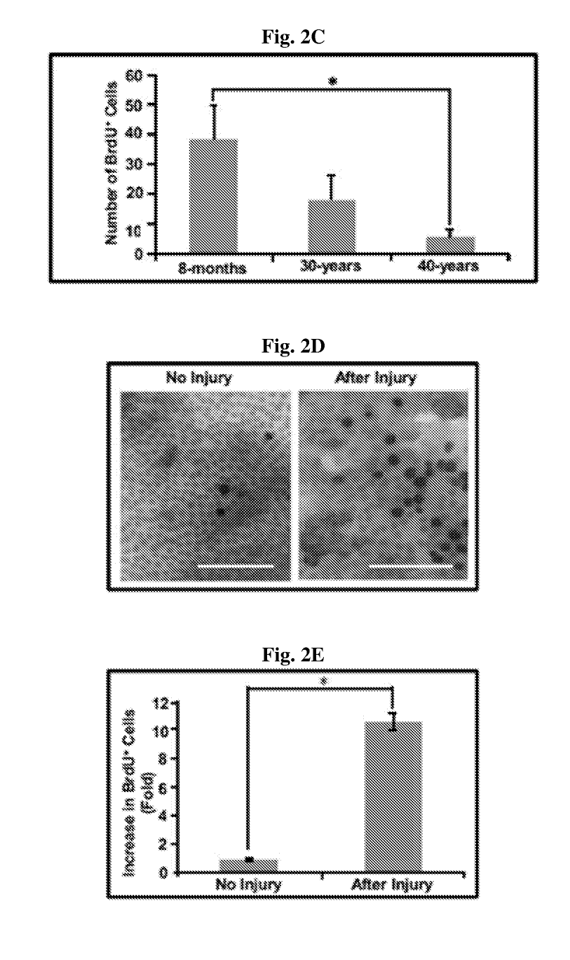

In the mature lens, LECs cover the anterior surface of the lens and begin to differentiate into lens fibers at the equator (FIG. 2A). Sustained self-renewal and protective capacities against external injury and oxidative damage are among the most significant functions of LECs. To assess the regenerative ability of LECs, bromodeoxyuridine (BrdU) labeling was used to identify proliferating LECs from human donor lenses. BrdU⁺ LECs were quantified in 8-month-, 30-year-, and 40-year-old donors and it was found that the number of proliferating cells decreased with age (FIG. 2B-FIG. 2C). However, upon surgical removal of the entire lens contents with preservation of the empty capsular bag scaffold, the number of BrdU⁺ cells increased by 11-fold (P<0.05, FIG. 2D-FIG. 2E), suggesting a strong regenerative capacity of human LECs after injury.

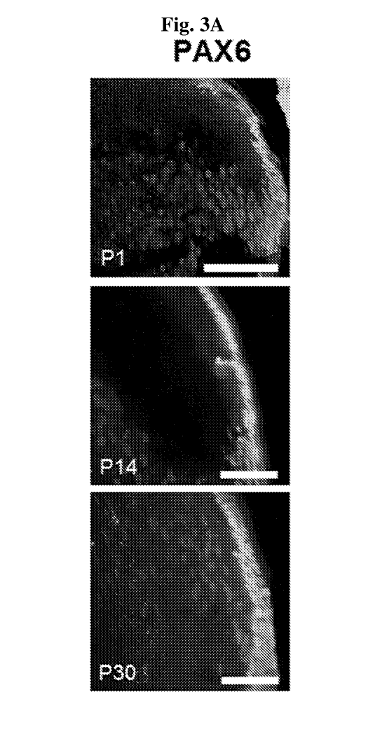

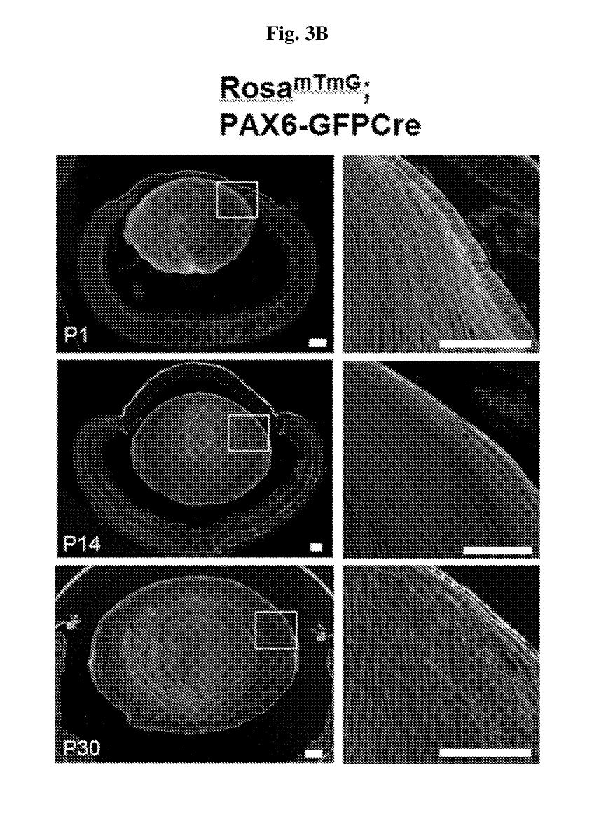

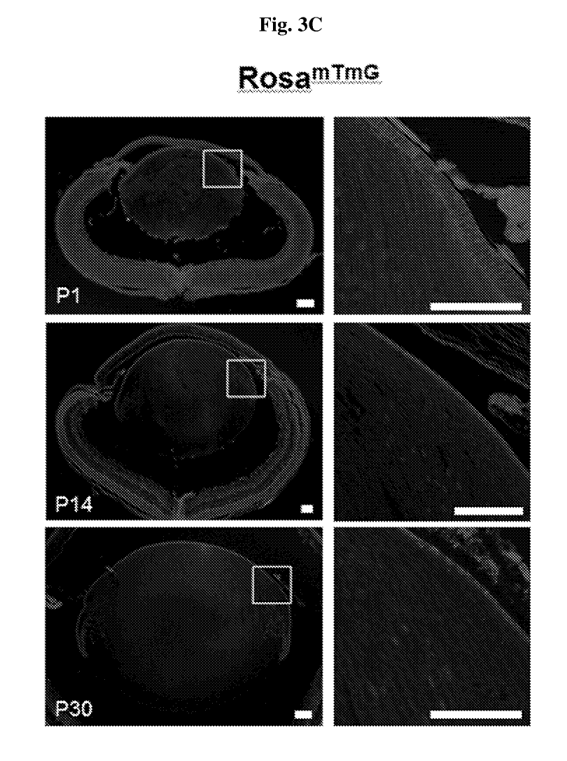

Pax6 plays a central role in eye development as well as in lens induction. After birth, Pax6 maintains a high level of expression in the lens epithelium, particularly at the germinative zone (FIG. 3A). To determine whether Pax6⁺ LECs can contribute to lens fiber cell formation, lineage-tracing experiments was performed in mice by crossing a Pax6 lens ectoderm enhancer-driven Cre deleter mouse strain (P0-3.9-GFPCre) with the ROSA^{mTmG} membrane-bound GFP reporter strain. Intense membrane GFP⁺ cells were observed throughout the entire lens of ROSA^{mTmG}; Pax6P0-3.9-GFPCre mice at P1, P14, and P30. In contrast, the P0-3.9-GFPCre allele alone yielded only nuclear GFP expression in LECs detectable by anti-GFP antibody staining (FIG. 3A-FIG. 3B). These results indicate that Pax6⁺ LECs from embryonic or adult lens contribute to the replacement of mouse lens fiber cells postnatally.

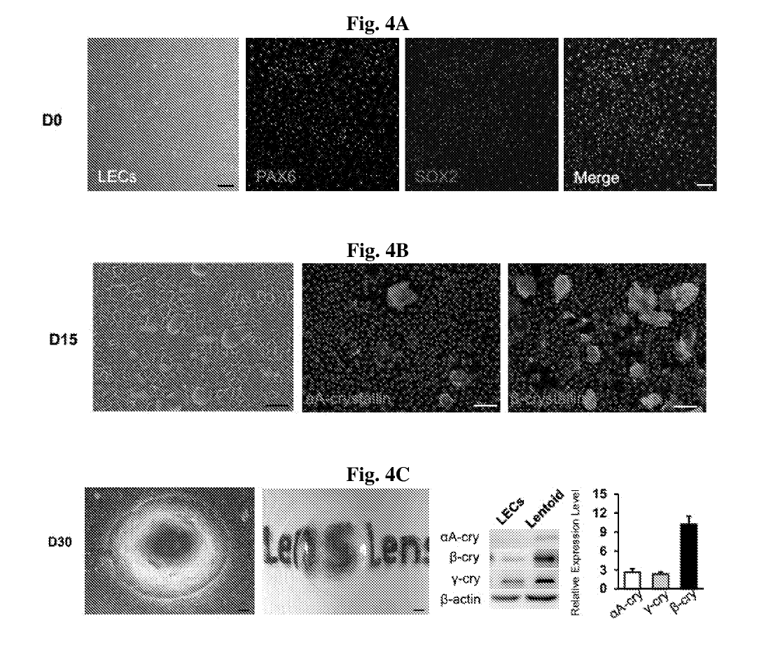

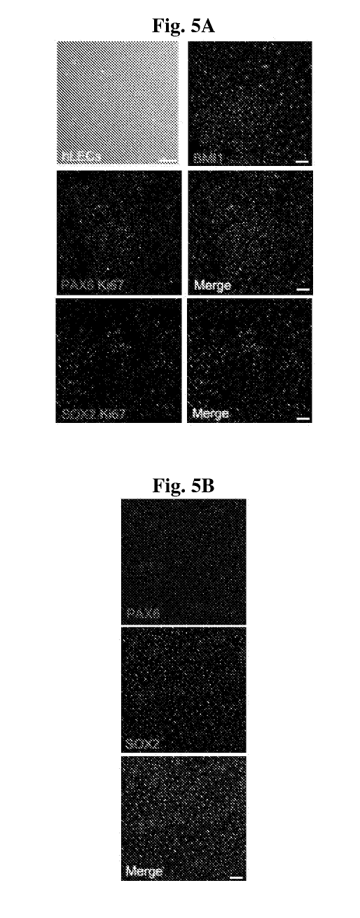

Rabbit LECs from neonatal lens capsules were isolated and expanded. These LECs showed a cobblestone-like epithelial morphology with highly positive staining for LECs markers Pax6 and Sox2, and could be passaged over time (FIG. 4A). Upon differentiation, these LECs formed transparent three-dimensional convex lens-like structures, defined as lentoid bodies (FIG. 4B-FIG. 4C), which possess significant refractive power (FIG. 4C). Immunostaining and Western blot analysis showed that lentoid bodies expressed mature lens fiber-specific genes, including those encoding αA-, β-, and γ-crystallins (FIG. 4B-FIG. 4C).

### Disruption of LEC Homeostasis And Integrity Leads to Cataract Formation

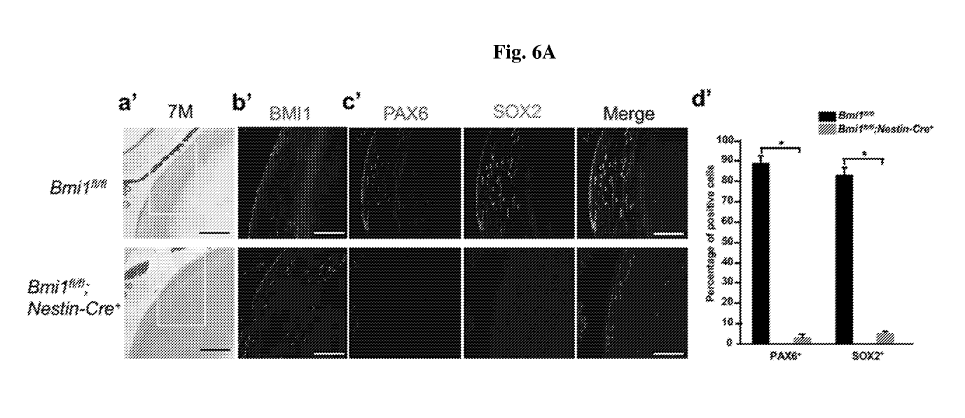

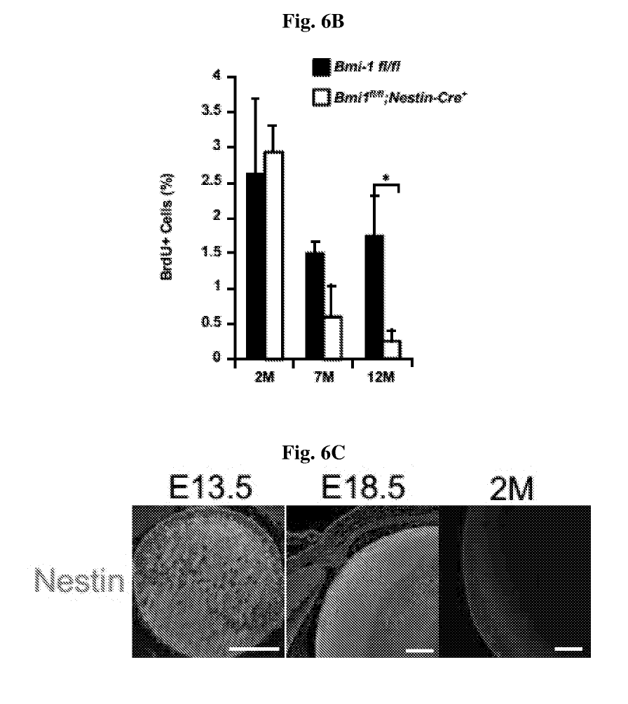

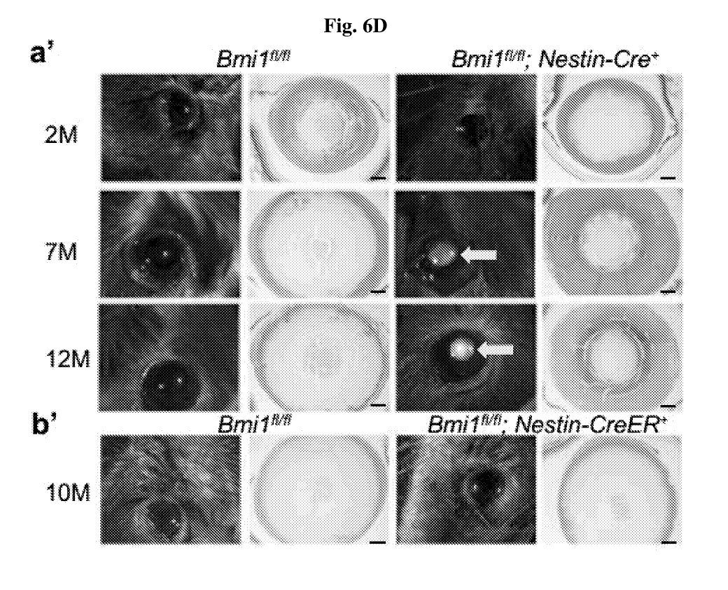

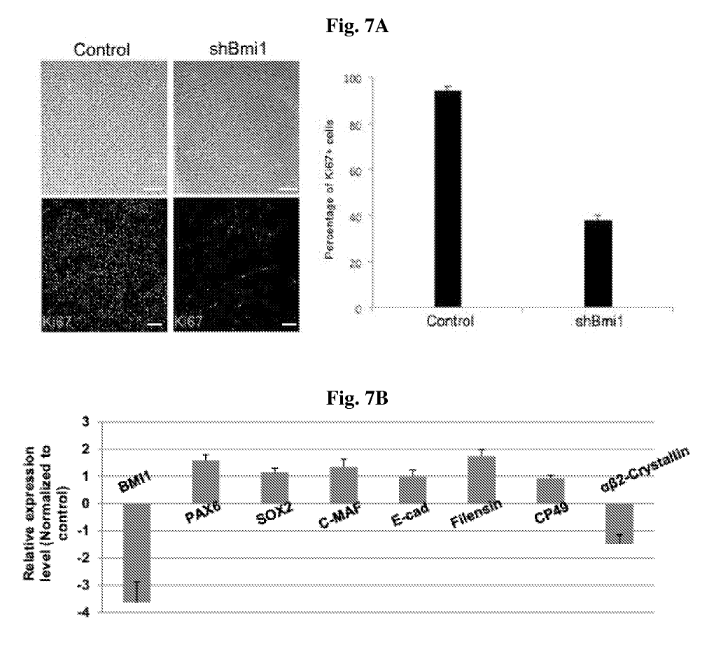

The LEC pool and its role in the maintenance of lens function was examined by studying, BMI-1, a member of the Polycomb-group family. BMI-1 is known to promote the maintenance and self-renewal of stem cells in multiple postnatal tissues and is expressed in both the murine lens germinative zone and in cultured human fetal LECs (FIG. 5A-FIG. 5B, FIG. 6A). Knockdown of BMI-1 in human LECs led to significantly decreased LECs proliferation in vitro (FIG. 7A), without affecting expression of key genes in LECs or lens fiber cells (FIG. 7B). To directly test the effects of conditional deletion of Bmi-1 on LEC proliferation, BrdU was administered to 2-, 7-, and 12-month-old Nestin-Cre;Bmi-1^{fl/fl} mice and Bmi-1^{fl/fl} littermate controls. After a 20-hour pulse, there was no significant difference in the percentage of BrdU⁺ LECs in 2-month-old Nestin-Cre;Bmi-1^{fl/fl} mice and Bmi-1^{fl/fl} controls. However, there was a significant reduction in the percentage of BrdU⁺ LECs in 7- and 12-month-old Nestin-Cre;Bmi-1^{fl/fl} eyes compared to controls (FIG. 6B, P<0.05).

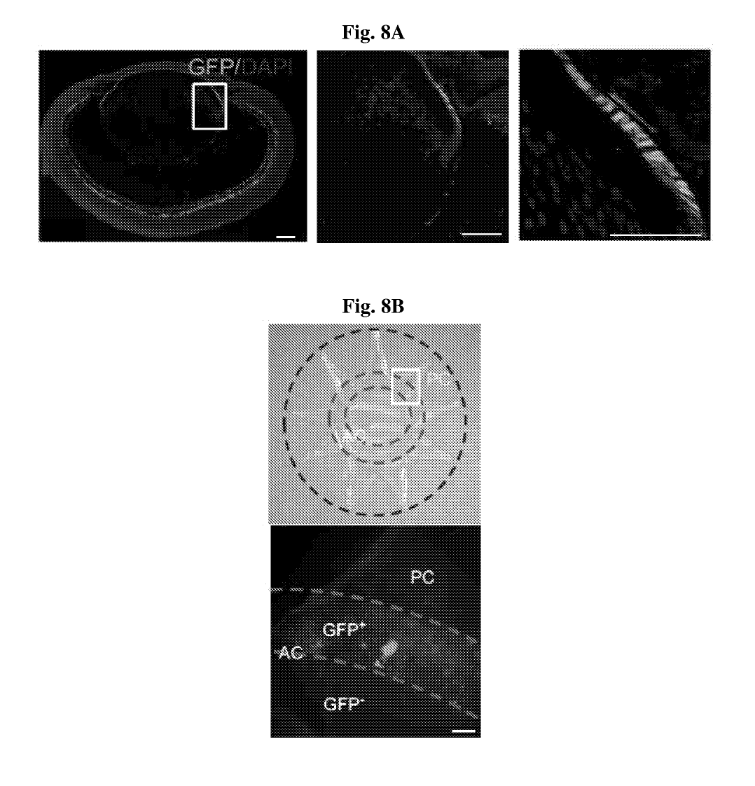

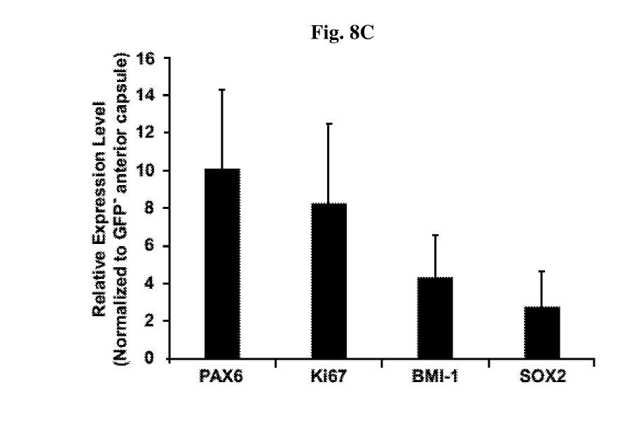

The mRNA expression levels of Bmi1, Sox2 and Ki67 in Pax6⁺ LECs were investigated at the anterior capsule in Pax6P0-3.9-GFPCre mouse lens. Compared with Pax6⁻ (GFP-negative) LECs, Pax6⁺ (GFP-positive) LECs located at the germinative zone had higher expression levels of Bmi1, Sox2 and Ki67 (FIG. 8A-FIG. 8C). Moreover, conditional deletion of Bmi-1 led to a dramatic decrease in the number of Pax6⁺/Sox2⁺ LECs in aging Nestin-Cre;Bmi-1^{fl/fl} mice (FIG. 6A, P<0.001). Additionally, the lenses of aging Nestin-Cre;Bmi-1^{fl/fl} mice became progressively opaque, suggesting cataract formation. To test this hypothesis, tropicamide drops was administered to the eyes of 2-, 7-, and 12-month-old Nestin-Cre;Bmi-1^{fl/fl} mice and Bmi-1^{fl/fl} littermate controls to dilate the pupils (FIG. 6C-FIG. 6D). Eyes of 2-month-old Nestin-Cre;Bmi-1^{fl/fl} mice (n=3) were indistinguishable from those of age-matched controls (n=4). However, 100% of the 7-month-old (n=5) and 12-month-old (n=7) Nestin-Cre;Bmi-1^{fl/fl} mice had bilateral cataracts, while none of the age-matched Bmi-1^{fl/fl} controls (n=3, 7-month-old; n=5, 12-month-old) developed cataracts. Moreover, H&E stained sections revealed the presence of cataracts in the 7- and 12-month-old Nestin-Cre;Bmi-1^{fl/fl} mice (FIG. 6D). These exemplify that Bmi-1 loss-of-function disrupted LEC proliferation, thereby depleting the LEC pool and promoting cataract formation.

### Preservation of LEC Integrity and Lens Regeneration Using Minimally Invasive Capsulorhexis Surgery

The current capsulorhexis method performed in pediatric cataract surgery involves making a large 6-mm diameter opening at the center of the anterior capsule, resulting in a large wound area and destruction of significant numbers of LECs (FIG. 1C). To overcome these limitations and to facilitate lens regeneration, a new capsulorhexis method was established. This new method has two advantages: 1) it reduces the size of the wound considerably, and 2) it moves the capsulorhexis opening from the central visual axis to the periphery. Thus, application of this procedure led to improved visual axis transparency and preservation of LECs with regenerative potential (FIG. 9A).

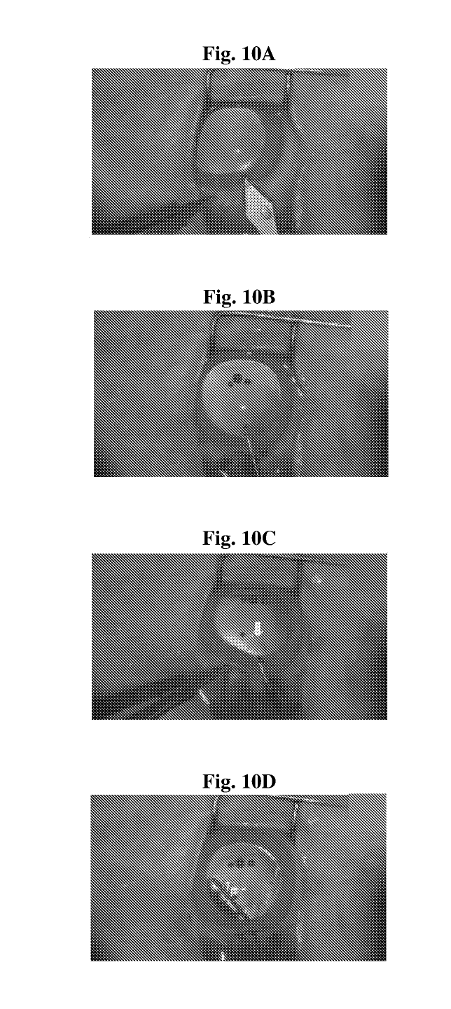

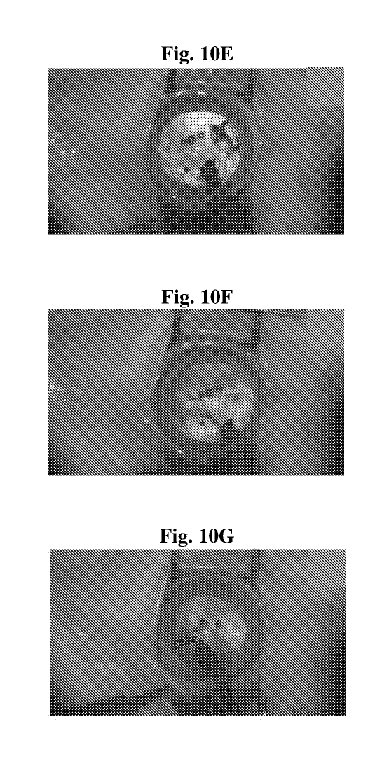

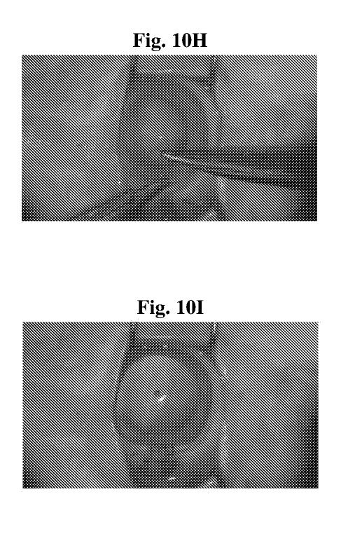

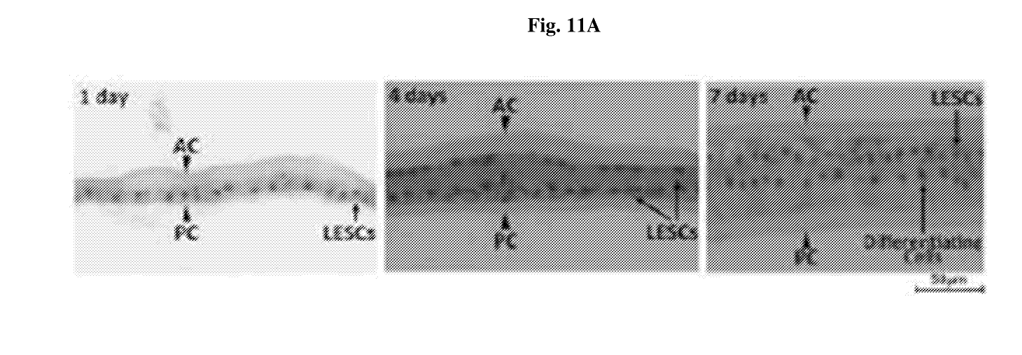

In vivo lens regeneration was investigated in rabbit eyes. A new minimally invasive capsulorhexis technique described herein was used to preserve endogenous LECs while removing the native lens (FIG. 10A-FIG. 10I). One day after surgery, slit-lamp microscopy showed that the anterior and posterior capsules were adherent (FIG. 9B). Four to five weeks after surgery, the regenerating lens tissue grew from the periphery of the capsular bag toward the center in a curvilinear symmetrical pattern (FIG. 9B). Seven weeks after surgery, the regenerating lens tissue formed a transparent biconvex lens along the anterior-posterior axis with a clear view of the posterior segment and retina (FIG. 9B-FIG. 9C), comparable to a normal healthy lens (FIG. 9C). The refractive power of the regenerated lenses after surgery was evaluated and found to have increased to an average of 15.6 diopters from the first to the fifth month after surgery, a value comparable to that of a normal lens²¹(FIG. 9D, P<0.01).

The LECs in the germinative zone of regenerated lenses showed intense proliferative activity 7 weeks post-surgery, as evidenced by both Ki67 and BrdU labeling (FIG. 9E-FIG. 9G). Notably, some PAX6⁺ LECs co-labeled with BrdU, demonstrating their proliferative potential (FIG. 9G). These LECs lost PAX6 expression concomitant with the initiation of differentiation and subsequent migration from the lens equator.

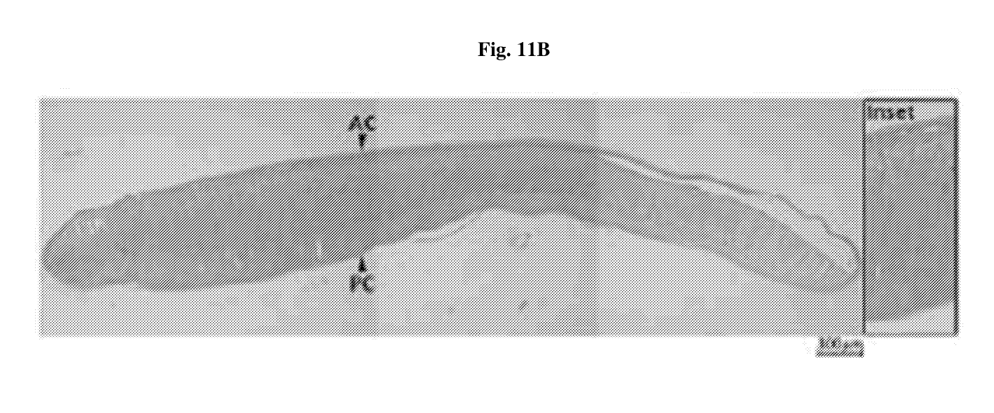

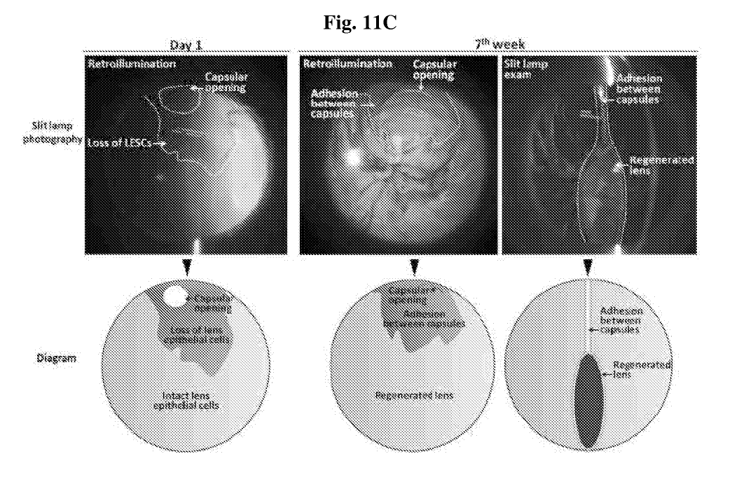

One day post-surgery, histological examination revealed that a monolayer of LECs remained intact (FIG. 11A). Four days post-surgery, LECs migrated onto the posterior capsule from the periphery toward the center in a curvilinear 360-degree fashion with a single layer of epithelium on the posterior capsule (FIG. 11A). Seven days post-surgery, LECs on the posterior capsule began to elongate, and their nuclei were positioned anteriorly away from the posterior capsule (FIG. 11A). Twenty-eight days post-surgery, a structure with lens fibers and an extruded nucleus was observed (FIG. 11B). At week 7 after surgery, the regenerated lens fibers elongated along the anterior-posterior axis and grew to cover the entire posterior capsular area, forming a lens with a double-convex shape (FIG. 11C).

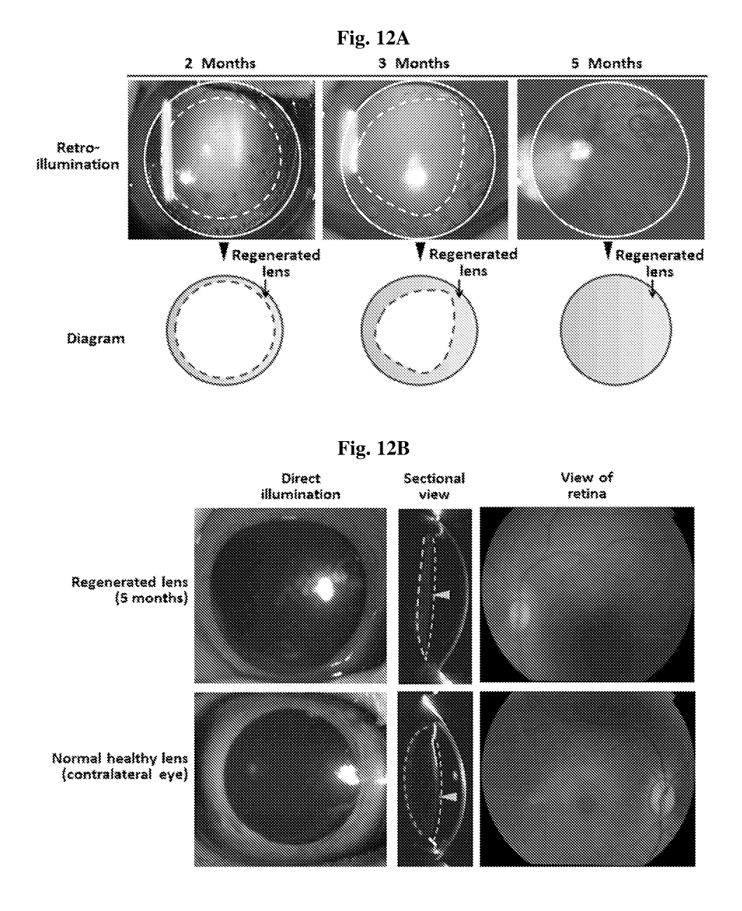

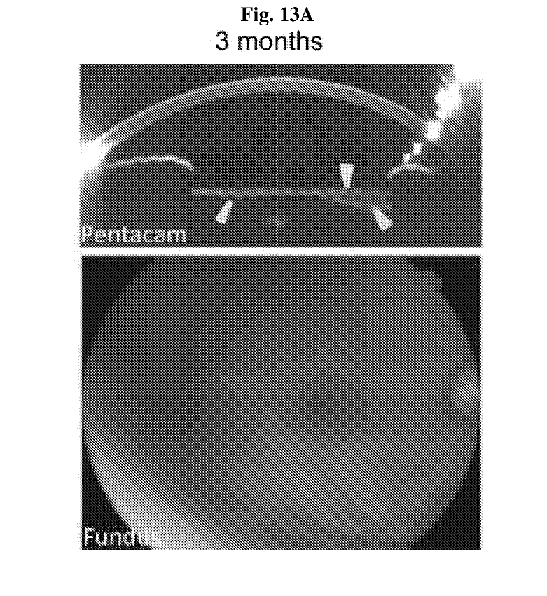

Lens regeneration was investigated in macaques 1-3 months of age (approximately equivalent to human infants 4-12 months old), using a similar minimally invasive surgical technique. From postoperative days 1 to 3, no signs of inflammation or other undesired side effects were seen. Two to three months post-surgery, regenerating lens tissue had grown from the periphery toward the center in a curvilinear pattern (FIG. 12A). Five months post-surgery, a biconvex lens with a transparent visual axis had formed (FIG. 12A-FIG. 12B). Fundus examination seven weeks after surgery showed a clear view of the retina, comparable to the view of the retina seen through a normal healthy lens. No undesired complications, such as macular edema, retinal detachment, or endophthalmitis were observed.

### Lens Regeneration in Human Infants

Cataract is a major cause of vision loss in human infants. Currently, the most commonly practiced surgical procedure involves removal of the cloudy lens through a large ACCC, combined with either posterior laser capsulotomy or PCCC and anterior vitrectomy (FIG. 1A-FIG. 1C), which is followed by artificial lens implantation or postoperative aphakic eyeglasses or contact lenses. However, complications such as visual axis opacity (VAO) often occur. Moreover, difficulty with refractive correction of developing eyes, secondary glaucoma, and surgery-related complications can lead to a poor outcome. A clinical trial was conducted in pediatric cataract patients up to two years of age to investigate whether lenses could be regenerated in humans using minimally invasive surgery.

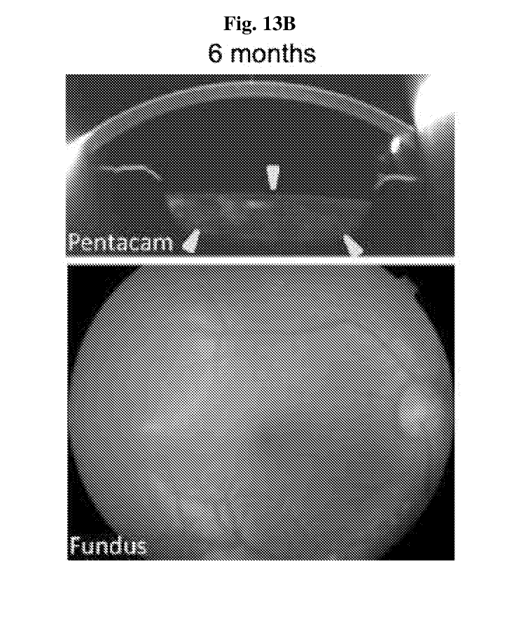

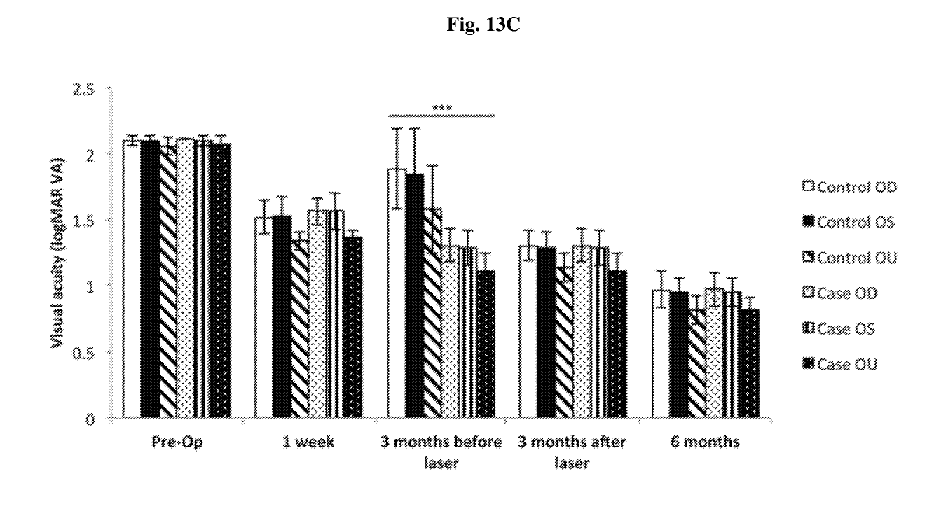

Twelve pediatric cataract patients (24 eyes) underwent minimally invasive surgery to promote lens regeneration, while 25 pediatric cataract patients (50 eyes) in the control group received the current standard-of-care treatment that left them aphakic. Using slit-lamp microscopy, we were able to dynamically observe and record the process of in vivo lens regeneration postoperatively. The capsular openings healed within one month after minimally invasive surgery. Three months post-surgery, a regenerated transparent biconvex lens structure had formed (FIG. 13A-FIG. 13B). No significant VAO or other complications were observed at 8 months post-surgery (Table 1 and Table 3).

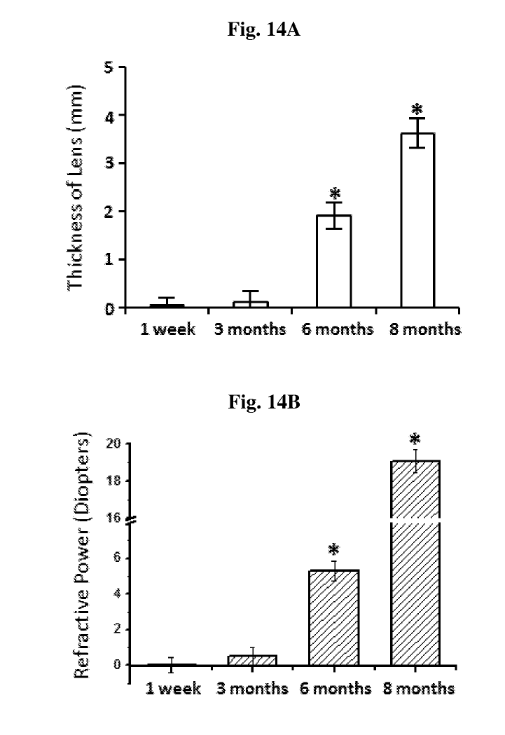

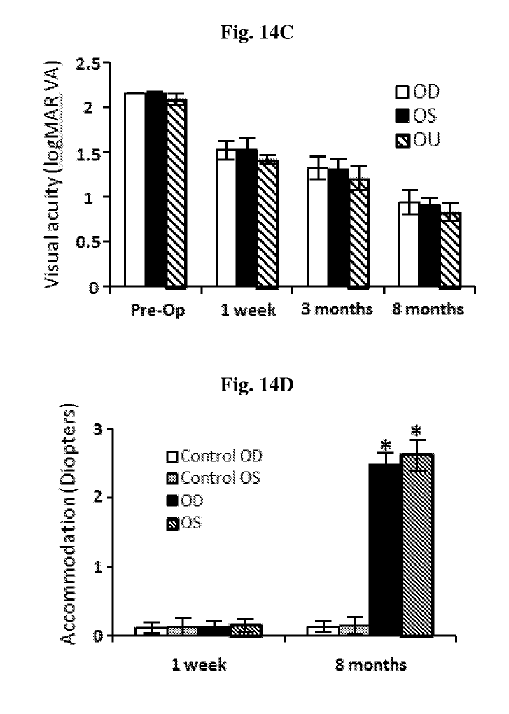

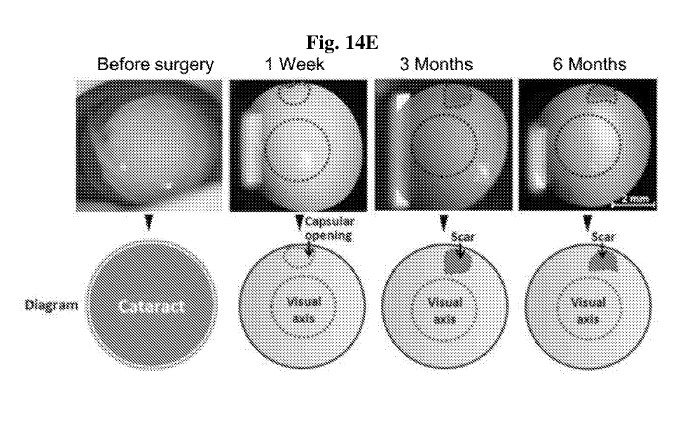

Slit-lamp microscopy with retroillumination and a Pentacam system were utilized to evaluate the functional properties of the regenerated lenses. All of the eyes gained visual function when the capsular bag was refilled with a regenerated lens of relatively uniform density. A clear view of the fundus was observed in all cases with successful lens regeneration (FIG. 13A-FIG. 13B). The average central thickness of the regenerated lenses increased significantly after surgery and was comparable to a native lens at 8 months post-surgery (FIG. 14A, P<0.01). Retinoscopy and ophthalmoscopy were also used to evaluate the function of the regenerated lenses and it was found that from the first week to 8 months post-surgery, the refractive power increased significantly (FIG. 14B, n=24, P<0.01).

The accommodative ability of the regenerated lenses was evaluated 8 months after surgery using an open-field autorefractor to measure accommodative responses at different distances and dynamic retinoscopy to validate the accommodative response. The mean accommodative response increased to 2.5 diopters in regenerated lenses, which was markedly improved compared to the 0.10 diopter increase in aphakic controls (*P<0.001). Using Teller Acuity Cards to compare pre- and postoperative visual acuity, the grating acuity (cycles/degree) was recorded preoperatively and at each postoperative follow-up appointment, and converted to the logarithm of the minimum angle of resolution (logMAR). Infant visual acuity and accommodation power were significantly improved postoperatively compared to the preoperative baseline (FIG. 14C-FIG. 14D). The increase in visual acuity was comparable to that achieved using the current surgical method (FIG. 13C). Thus, visual function testing showed that the regenerated lenses were functional.

### Clinical Outcome Comparison Between Minimally Invasive Surgery and Current Standard-of-Care Surgery

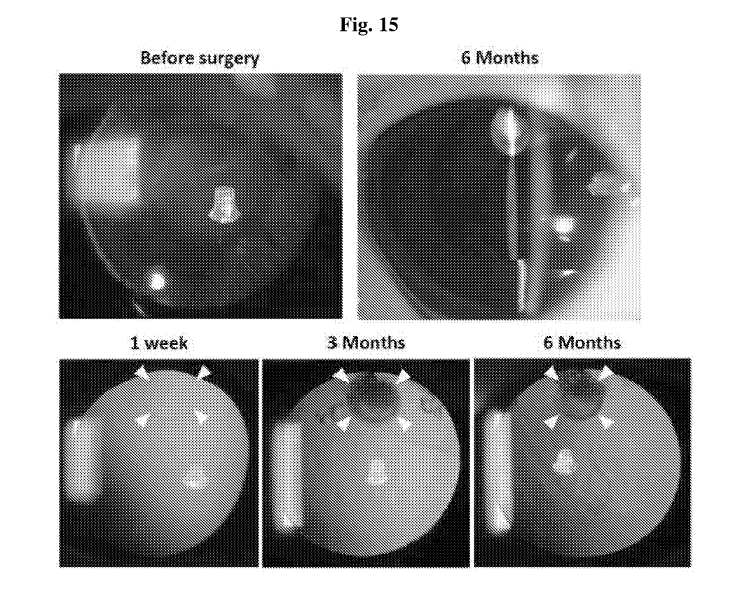

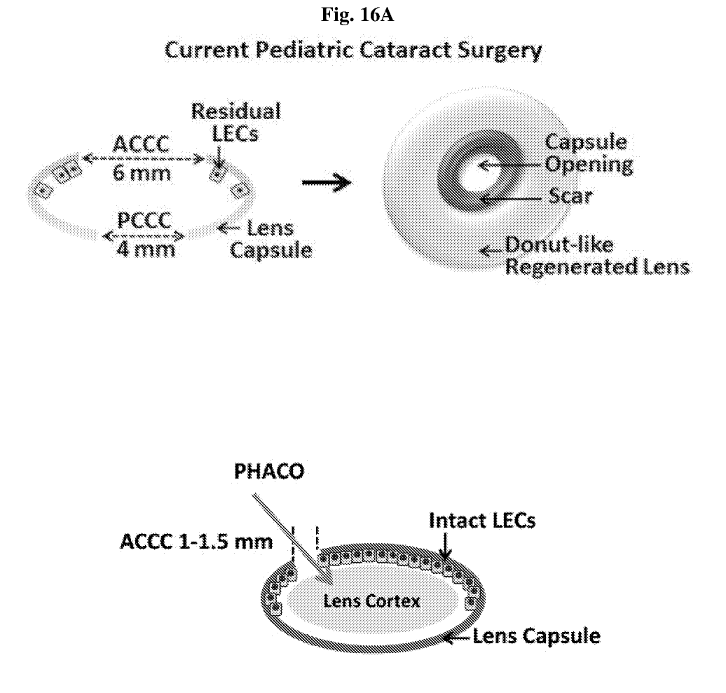

With the current method for pediatric cataract surgery, VAO will occur in nearly all patients weeks or months postoperatively due to the abnormal proliferation of residual LECs (Table 1 and Table 3). The younger the patient, the sooner it occurs. To avoid VAO, additional procedures such as polishing of the lens capsule, laser capsulotomy, PCCC, and anterior vitrectomy are widely practiced to disrupt LECs, the lens capsule on which LECs proliferate, and aberrant lens fiber regeneration. Although these procedures can decrease VAO incidence by 15%, they carry significant risk of postoperative inflammation and complications. In this clinical trial, the present minimally invasive surgical method resulted in visual axis transparency in nearly all eyes (95.8%) (FIG. 14E, FIG. 15, Table 1 and Table 3). Since the scar from the ACCC was <1.5 mm in diameter and located in the periphery of the anterior capsule, it was far from the visual axis (FIG. 14E) and not visible unless the pupils were dilated. The preserved lens capsule remained nearly entirely transparent (FIG. 14E). No disorganized tissue regeneration was observed. Thus, compared to the current standard-of-care for cataract surgery, the present new minimally invasive technique decreased VAO by more than 20-fold (84% vs. 4.2%). Furthermore, there was an intact posterior capsule and lens-vitreous interface (Table 1 and Table 3).

By using paired t-test within each group, significantly improvement of decimal acuity before and after treatment was observed with p-value<0.001 (t=23.40, df=49.04) in standard-of-care group and p-value<0.001 (t=15.05, df=23.01) in novel treatment group, respectively. A linear mixed-effect model using decimal acuity as outcome (time: baseline, 1 week, 3 month (after surgery for control group)) and treatment assignment and their interaction as fixed effects yielded statistically insignificant result for time and treatment interaction by likelihood ratio test with p-value 0.956(χ²=0.332, df=3) (Table 4A left, Table 4C left), which indicated the mean response profiles for two groups were parallel over time. The linear mixed-effect model was refit by dropping out the interaction term (Table 4B left). A likelihood ratio test with insignificant p-value 0.776 (χ²=0.081, df=1) (Table 4C left), illustrated the difference between mean decimal acuity in two groups were not statistically different over time (FIG. 20B). In contrast, the linear mixed-effect model using decimal acuity as outcome, time: baseline, 1 week, 3 month (before surgery for control group), treatment assignment and their interaction as fixed effects yielded statistically significant result for time and treatment interaction with p-value<0.001(χ²=47.529, df=3)(Table 4A right and Table 4C right). The non-parallel pattern of mean responses from two groups was largely due to the vision loss at 3 month before laser surgery in the control group, while the decimal acuity was monotonically increased in novel treatment group (FIG. 20B). The novel treatment also shows significantly lower complication rate by almost every measurement, supporting the superiority and safety of the novel treatment (Table 1 and Table 3).

Table 1 shows comparison of lens regeneration and complications in infants who received the new surgical technique versus the current technique.

| | Current treatment | | New treatment | | Odds ratio | P-value |
|---|---|---|---|---|---|---|
| Total patients | 25 | | 12 | | | |
| Total eyes | 50 | | 24 | | | |
| Regenerated lens structure | 0 | | 24 eyes | (100%) | | |
| Healing and closure of | 0 | | 24 eyes | (100%) | | |
| capsular openings | | | | | | |
| Decimal acuity (logMAR) | | | | | | |
| Pre-op | 0.017 | (2.1) | 0.009 | (2.1) | | P > 0.05 |
| 1 week | 0.044 | (1.3) | 0.041 | (1.4) | | P > 0.05 |
| | Before | | | | | Before |
| | laser: 0.034 (1.6) | | | | | laser: P < 0.001 |
| 3 months | After | | 0.075 | (1.1) | | After |
| | laser: 0.071 (1.1) | | | | | laser: P > 0.05 |
| 6 months | 0.15 | (0.8) | 0.15 | (0.8) | | P > 0.05 |
| Overall complication rate | 46 eyes | (92%) | 4 eyes | (16.7%) | 0.017 | P < 0.001 |
| | | | | | (0.004-0.077) | |
| Corneal edema | 15 eyes | (30%) | 2 eyes | (8.3%) | 0.212 | P = 0.04 |
| | | | | | (0.044-1.018) | |
| Anterior chamber | 37 eyes | (74%) | 4 eyes | (16.7%) | 0.070 | P < 0.001 |
| inflammation | | | | | (0.020-0.244) | |
| Macular edema | 3 eyes | (6%) | 0 | | | |
| Endophthalmitis | 0 | | 0 | | | |
| Retinal detachment | 0 | | 0 | | | |
| Ocular hypertension | 9 eyes | (18%) | 0 | | | |
| Visual axis opacification | 42 eyes | (84%) | 1 eye | (4.2%) | 0.008 | P < 0.001 |
| | | | | | (0.001-0.070) | |

| | | | |
|---|---|---|---|
| Additional laser surgery | 42 eyes | (84%) | 0 |
| Anterior vitrectomy | 8 eyes | (16%) | 0 |

Table 2 illustrates primers used for real-time PCR.

| Gene (Human) | Forward primer | Reverse primer |
|---|---|---|
| c-Maf | GCCCAACCTGGTGGCTGTGTGCCT | AGACACCAGGTCCGGGCTGGGGT |
| | (SEQ ID NO: 1) | GC |
| | | (SEQ ID NO: 2) |
| CP49 | GCTTGGAGCAAGGCTCCTGCTT | ACGTGAAGGTGCTGTACACAC |
| | (SEQ ID NO: 3) | (SEQ ID NO: 4) |
| E-cadherin | GACTTCGAGGCGAAGCAGCAGT | ATCTTCTGCTGCATGAATGTGTC |
| | (SEQ ID NO: 5) | (SEQ ID NO: 6) |
| filensin | GACCCTGGAACAAGCTAT | ATCCGATGGTACCGGTCCAGC |
| | (SEQ ID NO: 7) | (SEQ ID NO: 8) |
| GAPDH | GCGAGATCCCGCCAACATCAAGT | AGGATGCGTTGCTGACAATC |
| | (SEQ ID NO: 9) | (SEQ ID NO: 10) |
| Pax6 | GTATTCTTGCTTCAGGTAGAT | GAGGCTCAAATGCGACTTCAGCT |
| | (SEQ ID NO: 11) | (SEQ ID NO: 12) |
| Prox1 | GCTTTGCTTTTTTCAAGTGATT | AGGCTTCACCACGTCCACCTTCCG |
| | (SEQ ID NO: 13) | C |
| | | (SEQ ID NO: 14) |
| Sox2 | GAACGCCTTCATGGTGTGGT | AGCGTCTTGGTTTTCCGC |
| | (SEQ ID NO: 15) | (SEQ ID NO: 16) |
| βB2-crystallin | GCGAGTACCCTCGCTGGGACT | ACGACACCTTCTCCTGGTAGC |
| | (SEQ ID NO: 17) | (SEQ ID NO: 18) |
| Bmi1 | GGTACTTCATTGATGCCACAACC | CTGGTCTTGTGAACTTGGACATC |
| | (SEQ ID NO: 19) | (SEQ ID NO: 20) |
| Gene (Mouse) | Forward primer | Reverse primer |
| Bmi1 | ACTACACGCTAATGGACATTGCC | CTCTCCAGCATTCGTCAGTCCA |
| | (SEQ ID NO: 21) | (SEQ ID NO: 22) |
| GAPDH | CATCACTGCCACCCAGAAGACTG | ATGCCAGTGAGCTTCCCGTTCAG |
| | (SEQ ID NO: 23) | (SEQ ID NO: 24) |
| PAX6 | CTGAGGAACCAGAGAAGACAGG | CATGGAACCTGATGTGAAGGAGG |
| | (SEQ ID NO: 25) | (SEQ ID NO: 26) |
| SOX2 | AACGGCAGCTACAGCATGATGC | CGAGCTGGTCATGGAGTTGTAC |
| | (SEQ ID NO: 27) | (SEQ ID NO: 28) |
| Ki67 | ATCATTGACCGCTCCTTTAGGT | GCTCGCCTTGATGGTTCCT |
| | (SEQ ID NO: 29) | (SEQ ID NO: 30) |

Table 3A-Table 3C illustrate comparison of lens regeneration and complications in infants who received the new surgical treatment versus the current treatment.

TABLE 3A

| | Current treatment | New treatment |
|---|---|---|
| Total patients | 25 | 12 |
| Total eyes | 50 | 24 |
| Regenerated lens structure | 0 | 24 |
| Healing and closure of capsular | 0 | 24 |
| openings | | |

TABLE 3B

| | Current treatment decimal acuity | | New treatment decimal acuity | |
|---|---|---|---|---|
| | (standard deviation) | | (standard deviation) | |
| | OD | OS | OD | OS |
| Baseline | 0.008 (0.001) | 0.008 (0.001) | 0.008 (0) | 0.008 (0.001) |
| 1 week | 0.03 (0.009) | 0.03 (0.010) | 0.03 (0.007) | 0.03 (0.008) |
| 3 months (before laser) | 0.02 (0.017) | 0.02 (0.022) | 0.05 (0.014) | 0.05 (0.017) |
| 3 months (after laser) | 0.05 (0.013) | 0.05 (0.018) | — | — |
| 6 months | 0.11 (0.034) | 0.11 (0.025) | 0.10 (0.038) | 0.11 (0.027) |

TABLE 3C

| | Current | New | | |
|---|---|---|---|---|
| | treat- | treat- | Mean difference | |
| | ment | ment | (95% CI) | P value |
| Overall | 46 (0.92) | 4 (0.17) | 0.75 (0.57, 0.95) | <0.001 |
| complication rate | | | | |
| Corneal oedema | 15 (0.30) | 2 (0.08) | 0.22 (0.02, 0.42) | 0.04 |
| Anterior chamber | 37 (0.74) | 4 (0.17) | 0.57 (0.35, 0.80) | <0.001 |
| inflammation | | | | |
| Macular oedema | 3 (0.06) | 0 | 0.06 (−0.04, 0.16) | 0.22 |
| Endophthalmitis | 0 | 0 | — | — |
| Retinal detachment | 0 | 0 | — | — |
| Ocular hypertension | 9 (0.18) | 0 | 0.18 (0.04, 0.32) | 0.03 |
| Visual axis | 42 (0.84) | 1 (0.04) | 0.80 (0.64, 0.96) | <0.001 |
| opacification | | | | |
| Additional laser | 42 (0.84) | 0 | 0.84 (0.71, 0.97) | <0.001 |
| surgery | | | | |
| Anterior vitrectomy | 8 (0.16) | 0 | 0.16 (0.03, 0.29) | 0.04 |

Summary statistics of decimal acuity measured at each time point and complication in infants who received the new surgical technique versus the standard of care. Mean (standard deviation) is reported for continuous variables in the middle section (decimal acuity). OD, oculus dexter (right eye). OS, oculus sinister (left eye).

Table 4A to Table 4C shows clinical outcome analysis.

TABLE 4A
Linear mixed-effect model with decimal acuity as
outcome; time, treatment and their interaction as
fixed effects; and patient as random effect.

| | | | |
|---|---|---|---|
| Estimate | Std. Error | Z test | Pr(>\|Z\|) |

baseline, 1 week, 3 months after surgery and 6 months

| | | | | |
|---|---|---|---|---|
| (Intercept) | 0.008 | 0.003 | 2.97 | 0.003** |
| 1 week | 0.022 | 0.003 | 6.926 | <.001*** |
| 3 months | 0.042 | 0.003 | 13.409 | <.001*** |
| 6 months | 0.099 | 0.003 | 31.722 | <.001*** |
| Trmt (Novel) | 0 | 0.005 | −0.024 | 0.981 |
| 1 week*Trmt | −0.003 | 0.005 | −0.494 | 0.621 |
| 3 months*Trmt | 0 | 0.005 | −0.036 | 0.971 |
| 6 months*Trmt | −0.001 | 0.005 | −0.093 | 0.926 |
| Random effect | 0.008 | | | |
| −2logL | 1509.948 | | | |

baseline, 1 week, 3 months before surgery and 6 months

| | | | | |
|---|---|---|---|---|
| (Intercept) | 0.008 | 0.003 | 2.781 | 0.005** |
| 1 week | 0.022 | 0.003 | 6.859 | <.001*** |
| 3 months | 0.011 | 0.003 | 3.35 | <.001*** |
| 6 months | 0.099 | 0.003 | 31.417 | <001*** |
| Trmt(Novel) | 0 | 0.005 | −0.023 | 0.982 |
| 1 week*Trmt | −0.003 | 0.006 | −0.49 | 0.624 |
| 3 months*Trmt | 0.031 | 0.006 | 5.619 | <.001*** |
| 6 months*Trmt | −0.001 | 0.006 | −0.092 | 0.927 |
| Random effect | 0.01 | | | |
| −2logL | 1497.237 | | | |

TABLE 4B
Linear mixed-effect model with decimal acuity as outcome; time
and treatment as fixed effect; and patient as random effect.

| | | | |
|---|---|---|---|
| Estimate | Std. Error | Z test | Pr(>\|Z\|) |

baseline, 1 week, 3 months after surgery and 6 months

| | | | | |
|---|---|---|---|---|
| (Intercept) | 0.008 | 0.003 | 3.346 | <.001*** |
| 1 week | 0.021 | 0.003 | 8.126 | <.001*** |
| 3 months | 0.042 | 0.003 | 16.374 | <.001*** |
| 6 months | 0.099 | 0.003 | 38.731 | <001*** |
| Trmt (Novel) | −0.001 | 0.004 | −0.276 | 0.783 |
| Random effect | 0.008 | | | |
| −2IogL | 1536.077 | | | |

baseline, 1 week, 3 months before surgery and 6 months

| | | | | |
|---|---|---|---|---|
| (Intercept) | 0.006 | 0.003 | 2.107 | 0.035* |
| 1 week | 0.021 | 0.003 | 7.347 | <.001*** |
| 3 months | 0.021 | 0.003 | 7.313 | <.001*** |
| 6 months | 0.099 | 0.003 | 35.017 | <.001*** |
| Trmt (Novel) | 0.007 | 0.004 | 1.746 | 0.081 |
| Random effect | 0.009 | | | |
| −2IogL | 1476.647 | | | |

TABLE 4C
Likelihood ratio test of fixed effects based
on the analysis of response profiles.

| | | |
|---|---|---|
| DF | Chi-Squared | P-value |

baseline, 1 week, 3 months after surgery and 6 months

| | | | |
|---|---|---|---|
| Time*Treatment | 3 | 0.322 | 0.956 |
| Time | 3 | 532.308 | <.001*** |
| Treatment | 1 | 0.081 | 0.776 |

baseline, 1 week, 3 months before surgery and 6 months

| | | | |
|---|---|---|---|
| Time*Treatment | | 47.529 | <.001*** |
| Time | 3 | 495.562 | <.001*** |
| Treatment | 1 | 3.089 | 0.079 |

### Example 2—Biomaterial Composition to Induce Proliferation and Differentiation of Lens Epithelial Stem and Progenitor Cells

### A Minimally Invasive Capsulorhexis Surgery Method to Deliver Biomaterial Composition

The capsulorhexis surgery method disclosed herein is used to deliver a biomaterial composition to maintain the structural integrity of the lens anterior capsule of the eye and to induce expansion of lens epithelial stem and progenitor cells. The biomaterial composition comprises of human serum and fibroblast growth factor (FGF). In some instances, the biomaterial composition optionally includes one or more nutrients and additive. In some instances, the one or more nutrients comprise a composition of amino acids. In some instances, the one or more nutrients comprise a glucose source. In some instances, the one or more nutrients comprise vitamins such as folic acid, nicotinamide, riboflavin, B₁₂, choline chloride, myo-inositol, niacinamide, D-Pantothenic acid, Pyridoxal-HCl, thiamine-HCl, and the like. In some instances, the biomaterial composition optionally includes non-essential amino acids consisting of alanine, arginine, asparagine, aspartic acid, cysteine, glutamic acid, glutamine, glycine, proline, serine, and tyrosine. In some instances, the additives comprise inorganic salts such as calcium chloride, potassium chloride, magnesium sulfate, sodium chloride, monosodium phosphate, potassium phosphate, sodium bicarbonate, and sodium phosphate.

First, the size of the capsulorhexis opening is decreased to 1.0-1.5 mm in diameter. This results in a minimal wound of about 1.2 mm² in area, which is only about 4.3% the size of the wound created by the current method. Second, the location of the capsulorhexis is moved to the peripheral area of the lens instead of the central area. A 0.9 mm phacoemulsification probe is used to remove the lens contents and/or cortical opacities followed by the administration of the biomaterial composition in the range of 0.1× to 10× concentration. In some instances, the biomaterial composition is administered in 1X concentration.

The use of the biomaterial composition disclosed herein reduces the visual axis opacification (VAO) when compared to the method comprising a capsulorhexis procedure comprising central capsulorhexis opening and implantation of an artificial intraocular lens (TOL).

The use of the biomaterial composition disclosed herein results in lowered incidents of complications, such as corneal edema, anterior chamber inflammation, and visual axis opacification (VAO).

Minimally Invasive Capsulorhexis Surgery Method to Deliver Biomaterial Composition in Human Infants with Congenital Cataract

Pediatric patients are selected from the Childhood Cataract Program of the Chinese Ministry of Health (CCPMOH), which includes a series of studies on the influence of early interventions on the long-term outcomes of pediatric cataract treatment (ClinicalTrials.gov Identifier: NCT01844258). Inclusion criteria are the following: Infants are ≤24 months old, and diagnosed with bilateral or unilateral uncomplicated congenital cataract with an intact non-fibrotic capsular bag. Exclusion criteria included preoperative intraocular pressure (IOP)>21 mmHg, premature birth, family history of ocular disease, ocular trauma, or other abnormalities, such as microcornea, persistent hyperplastic primary vitreous, rubella, or Lowe syndrome. Twelve pediatric cataract patients (24 eyes) receive the new minimally invasive lens surgery alone with the biomaterial composition. Twenty-five pediatric cataract patients (50 eyes in total) are enrolled as the control group to receive the current standard surgical treatment.

The incidence of corneal edema is defined as >5% increase in central corneal thickness one week post-surgery, and the incidence of severe anterior chamber inflammation as Flare value>10 evaluated by Pentacam system (OCULUS, Germany) and Laser flare meter (KOWA FM-600, Japan). Early-onset ocular hypertension is identified as TOP>21 mmHg by Tonopen (Reichert, Seefeld, Germany) within 1 month after surgery. Macular edema is identified by fundus OCT (iVue, Optovue, Germany) as an increase in central macular thickness>10% one week post-surgery. When indicated, VAO, defined by visual decline and the degree to which the fundus is obscured, is treated with YAG laser capsulotomy at follow-up.

Compared to infants operated on using the new surgical technique described herein along with a biomaterial composition, infants who receive the traditional technique have a higher incidence of anterior chamber inflammation one week after surgery, early-onset ocular hypertension, and increased VAO. In the group treated with the present new method, a transparent regenerated biconvex lens is found in higher percentage of eyes 3 months after surgery, when compared to the group treated with the standard technique. In addition, higher percentage of the capsular openings heal within 1 month after surgery in the experimental group, compared to capsular openings in the control group.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Elements or components shown with any embodiment herein are exemplary for the specific embodiment and may be used on or in combination with other embodiments disclosed herein. While the invention is susceptible to various modifications and alternative forms, specific examples thereof have been shown in the drawings and are herein described in detail. The invention is not limited, however, to the particular forms or methods disclosed, but to the contrary, covers all modifications, equivalents and alternatives thereof.

What is claimed is:

1. Use of a biomaterial composition to maintain the structural integrity of a lens anterior capsule of an eye of a subject and to induce expansion of lens epithelial stem and progenitor cells in situ, wherein the biomaterial composition is administered into the lens anterior capsule through an capsulorhexis opening located at a peripheral area of the lens anterior capsule, and wherein the contents of the lens is removed prior to administration of the biomaterial composition.

2. The use of claim 1, wherein the biomaterial composition comprises human serum and a fibroblast growth factor (FGF).

3. The use of claim 1 or 2, wherein the biomaterial composition further comprises a nutrient, an additive, or a combination thereof.

4. The use of claim 3, wherein the nutrient comprises a composition of amino acids and optionally one or more nutrients.

5. The use of claim 3, wherein the additive comprises calcium chloride, potassium chloride, magnesium sulfate, sodium chloride, monosodium phosphate, potassium phosphate, sodium bicarbonate, sodium phosphate, or a combination thereof.

6. The use of claim 1, wherein the biomaterial composition is administered in a volume sufficient to replace the volume lost due to the removal of the contents of the lens from the lens anterior capsule.

7. The use of claim 1, wherein the capsulorhexis opening is about 1.0 to 2.0 mm in diameter.

8. The use of claim 1, wherein the capsulorhexis opening is about 1.0 to 1.5 mm in diameter.

9. The use of claim 1, wherein the capsulorhexis opening is located away from the central visual axis of the eye.

10. The use of claim 1, wherein the subject has cataract.

11. The use of claim 1, wherein the subject is an animal or human.

12. The use of claim 11, wherein the human is aged 18 or older.

13. The use of claim 11, wherein the human is aged 17 or younger.

14. The use of claim 13, wherein the human has a pediatric cataract.

15. The use of claim 11, wherein the human is an adult or an infant.

16. The use of claim 15, wherein the human infant has congenital cataract.

17. The use of claim 10, wherein cataract is removed.

18. The use of claim 1, wherein the lens epithelial stem and progenitor cells express Pax6 and/or Bmi-1.

19. The use of claim 1, wherein the use does not involve an implantation of an artificial intraocular lens (IOL).

20. The use of claim 1, wherein the use results in reduced visual axis opacification (VAO) relative to a use comprising a capsulorhexis procedure comprising central capsulorhexis opening and implantation of an artificial intraocular lens.

21. The use of claim 1, wherein the use results in lowered incidents of complications selected from the group consisting of corneal edema, anterior chamber inflammation, and visual axis opacification.

22. A system for performing a minimally invasive method of cataract removal, comprising an imaging unit, a phacoemulsification unit for emulsifying cataract material, an aspiration unit for removing cataract material, and a biomaterial delivery unit for delivering a biomaterial composition into a capsular bag via a lens capsule opening, wherein all of the units are operationally connected to a computer.

23. The system of claim 22, wherein the phacoemulsification unit comprises an ultrasound or laser probe, said probe is equipped with a tip designed to be inserted into a peripheral area of lens anterior capsule of an eye.

24. The system of claim 23, wherein the tip is configured to perform one or both of making an opening of about 1.0 to 2.0 mm in diameter and removing cataract from the eye.

25. The system of claim 23, wherein the tip is configured to perform one or both of making an opening of about 1.0 to 1.5 mm in diameter and removing cataract from the eye.

26. The system of claim 23, wherein the tip is configured to prevent damage to endogenous lens epithelial stem and progenitor cells.

27. The system of claim 22, wherein the imaging unit employs imaging technique selected from the group consisting of 3D imaging, optical coherence tomography, MRI, CT, and ultrasound.

28. The system of claim 22, wherein the biomaterial composition comprises human serum and a fibroblast growth factor (FGF).

29. The system of claim 22, wherein the biomaterial composition further comprises a nutrient, an additive, or a combination thereof.

30. The system of claim 29, wherein the nutrient comprises a composition of amino acids and optionally one or more nutrients.

31. The system of claim 29, wherein the additive comprises calcium chloride, potassium chloride, magnesium sulfate, sodium chloride, monosodium phosphate, potassium phosphate, sodium bicarbonate, sodium phosphate, or a combination thereof.

32. The system of claim 22, wherein the biomaterial composition is administered in a volume sufficient to replace the volume lost due to the removal of the cataract material from the capsular bag.


SEQUENCE LISTING (32 sequences)

SEQ ID NO: 1 - DNA, 24 bp, Artificial Sequence
  Description of Artificial Sequence Synthetic primer
gcccaacctggtggctgtgtgcct

SEQ ID NO: 2 - DNA, 25 bp, Artificial Sequence
  Description of Artificial Sequence Synthetic primer
agacaccaggtccgggctggggtgc

SEQ ID NO: 3 - DNA, 22 bp, Artificial Sequence
  Description of Artificial Sequence Synthetic primer
gcttggagcaaggctcctgctt

SEQ ID NO: 4 - DNA, 21 bp, Artificial Sequence
  Description of Artificial Sequence Synthetic primer
acgtgaaggtgctgtacacac

SEQ ID NO: 5 - DNA, 22 bp, Artificial Sequence
  Description of Artificial Sequence Synthetic primer
gacttcgaggcgaagcagcagt

SEQ ID NO: 6 - DNA, 23 bp, Artificial Sequence
  Description of Artificial Sequence Synthetic primer
atcttctgctgcatgaatgtgtc

SEQ ID NO: 7 - DNA, 18 bp, Artificial Sequence
  Description of Artificial Sequence Synthetic primer
gaccctggaacaagctat

SEQ ID NO: 8 - DNA, 21 bp, Artificial Sequence
  Description of Artificial Sequence Synthetic primer
atccgatggtaccggtccagc

SEQ ID NO: 9 - DNA, 23 bp, Artificial Sequence
  Description of Artificial Sequence Synthetic primer
gcgagatcccgccaacatcaagt

SEQ ID NO: 10 - DNA, 20 bp, Artificial Sequence
  Description of Artificial Sequence Synthetic primer
aggatgcgttgctgacaatc

SEQ ID NO: 11 - DNA, 21 bp, Artificial Sequence
  Description of Artificial Sequence Synthetic primer
gtattcttgcttcaggtagat

SEQ ID NO: 12 - DNA, 23 bp, Artificial Sequence
  Description of Artificial Sequence Synthetic primer
gaggctcaaatgcgacttcagct

SEQ ID NO: 13 - DNA, 22 bp, Artificial Sequence
  Description of Artificial Sequence Synthetic primer
gctttgcttttttcaagtgatt

SEQ ID NO: 14 - DNA, 25 bp, Artificial Sequence
  Description of Artificial Sequence Synthetic primer
aggcttcaccacgtccaccttccgc

SEQ ID NO: 15 - DNA, 20 bp, Artificial Sequence
  Description of Artificial Sequence Synthetic primer
gaacgccttcatggtgtggt

SEQ ID NO: 16 - DNA, 18 bp, Artificial Sequence
  Description of Artificial Sequence Synthetic primer
agcgtcttggttttccgc

SEQ ID NO: 17 - DNA, 21 bp, Artificial Sequence
  Description of Artificial Sequence Synthetic primer
gcgagtaccctcgctgggact

SEQ ID NO: 18 - DNA, 21 bp, Artificial Sequence
  Description of Artificial Sequence Synthetic primer
acgacaccttctcctggtagc

SEQ ID NO: 19 - DNA, 23 bp, Artificial Sequence
  Description of Artificial Sequence Synthetic primer
ggtacttcattgatgccacaacc

SEQ ID NO: 20 - DNA, 23 bp, Artificial Sequence
  Description of Artificial Sequence Synthetic primer
ctggtcttgtgaacttggacatc

SEQ ID NO: 21 - DNA, 23 bp, Artificial Sequence
  Description of Artificial Sequence Synthetic primer
actacacgctaatggacattgcc

SEQ ID NO: 22 - DNA, 22 bp, Artificial Sequence
  Description of Artificial Sequence Synthetic primer
ctctccagcattcgtcagtcca

SEQ ID NO: 23 - DNA, 23 bp, Artificial Sequence
  Description of Artificial Sequence Synthetic primer
catcactgccacccagaagactg

SEQ ID NO: 24 - DNA, 23 bp, Artificial Sequence
  Description of Artificial Sequence Synthetic primer
atgccagtgagcttcccgttcag

SEQ ID NO: 25 - DNA, 22 bp, Artificial Sequence
  Description of Artificial Sequence Synthetic primer
ctgaggaaccagagaagacagg

SEQ ID NO: 26 - DNA, 23 bp, Artificial Sequence
  Description of Artificial Sequence Synthetic primer
catggaacctgatgtgaaggagg

SEQ ID NO: 27 - DNA, 22 bp, Artificial Sequence
  Description of Artificial Sequence Synthetic primer
aacggcagctacagcatgatgc

SEQ ID NO: 28 - DNA, 22 bp, Artificial Sequence
  Description of Artificial Sequence Synthetic primer
cgagctggtcatggagttgtac

SEQ ID NO: 29 - DNA, 22 bp, Artificial Sequence
  Description of Artificial Sequence Synthetic primer
atcattgaccgctcctttaggt

SEQ ID NO: 30 - DNA, 19 bp, Artificial Sequence
  Description of Artificial Sequence Synthetic primer
gctcgccttgatggttcct

SEQ ID NO: 31 - DNA, 29 bp, Artificial Sequence
  Description of Artificial Sequence Synthetic oligonucleotide
aatgccatattggtatatgacataacagg

SEQ ID NO: 32 - DNA, 29 bp, Artificial Sequence
  Description of Artificial Sequence Synthetic oligonucleotide
gtaagaatcagatggcattatgcttgttg